(19) United States
(12) Patent Application Publication
(10) Pub. No.: US 2026/0211104 A1
(43) Pub. Date: Jul. 23, 2026

(54) CHILD PRESENCE DETECTION WITH CONFIGURABLE SENSOR FUSION FOR AUTONOMOUS AND SEMI-AUTONOMOUS MACHINES AND APPLICATIONS

(71) Applicant: NVIDIA CORPORATION, Santa Clara, CA (US)

(72) Inventors: Hairong Jiang, San Jose, CA (US); Sakthivel Sivaraman, San Jose, CA (US); Varsha Chandrashekhar Hedau, Sunnyvale, CA (US); Ratin Kumar, Cupertino, CA (US); Niranjan Avadhanam, Saratoga, CA (US)

(21) Appl. No.: 19/033,235

(22) Filed: Jan. 21, 2025

Related U.S. Application Data
(63) Continuation of application No. 19/033,188, filed on Jan. 21, 2025.

(51) Int. Cl. G01S 13/86; A61B 5/00; A61B 5/05; B60N 2/26; G01S 13/04; G06F 30/20

(52) U.S. Cl. CPC G01S 13/867; A61B 5/05; A61B 5/6893; B60N 2/266; G01S 13/04; G06F 30/20; A61B 2503/06

(57) ABSTRACT

Some embodiments apply occupant slot-dependent sensor selection, sensor fusion, and/or detection logic for child presence detection. In some embodiments, sensor fusion logic in a base control stack (e.g., a drive stack) of an ego-machine compute platform (e.g., a vehicle compute platform) may use configurable weights, thresholds, flags, values, and/or other parameters to customize whether and how data from multiple sensors or corresponding detection pipelines (e.g., raw sensor data, processed sensor data, computer vision or deep learning perception data) is combined. For example, camera detection result(s) and RADAR detection result(s) may be combined using configurable (e.g., slot-dependent) weights to generate a fused or unified child (and/or adult) presence classification (e.g., a binary result, detection confidence). As such, a sensor fusion algorithm in a base control (e.g., drive) stack may be configured (e.g., by an original equipment manufacturer) to customize (e.g., slot-dependent) sensor selection and/or sensor fusion logic for child presence detection.

### CROSS-REFERENCE TO RELATED APPLICATIONS

This application is a continuation of and claims priority to U.S. application Ser. No. 19/033,188, filed on Jan. 21, 2025, the contents of which are hereby incorporated by reference in their entirety.

### BACKGROUND

Sensor fusion combines data from multiple sensors —such as cameras, RADAR sensors, or LiDAR sensors —to create a more comprehensive representation of the environment being monitored, and may be used by ego-machines such as autonomous vehicles to facilitate more robust perception and decision-making. For example, autonomous vehicles use sensor fusion for tasks like object detection to improve the accuracy of the vehicle's perception system. Integrating data from different types of sensors helps compensate for the limitations of individual sensors, providing more reliable information under various conditions such as poor lighting or inclement weather.

In autonomous vehicles, sensor fusion is performed on a vehicle compute platform like the NVIDIA DRIVE AGX™ platform, which combines high-performance hardware and specialized software called a driving software stack (or simply a drive stack) to enable autonomous driving capabilities. The base drive stack that comes with the platform is optimized to run efficiently on the platform's hardware and provide functions like sensor fusion, obstacle detection, and path planning with minimal latency. The vehicle application layer sits on top of the base drive stack and allows Original Equipment Manufacturers (OEMs) to introduce custom logic and brand-specific features tailored to their vehicles.

There are trade-offs between performing sensor fusion in the base drive stack as-is versus integrating custom OEM logic through an application programming interface (API) in the vehicle application layer. Running sensor fusion in the base drive stack offers computational efficiency, as the software is optimized for the hardware platform, resulting in lower latency, better power management, and streamlined data processing. On the other hand, OEMs may prefer to perform sensor fusion using custom logic in the vehicle application layer. However, this customization can introduce additional layers of complexity, increasing computational overhead and reducing efficiency compared to the base stack. It can also lead to increased power consumption and latency due to the integration of additional software components that are not as tightly optimized with the underlying hardware. The choice often requires balancing efficiency and real-time performance with the desire for customization and control over the vehicle's perception system.

One of the challenges for the developer of a vehicle compute platform is to provide software that works for OEMs who often have different preferences for which sensor signals to use in different scenarios. Taking child presence detection as an example, one way in which child presence detection may be used involves detecting and alerting vehicle owners that an unattended child below a threshold age is present in an idle vehicle. As such, child presence detection may involve assessing the age of an individual (e.g., in an idle vehicle). One of the primary challenges in child presence and/or age detection arises out of the wide variety of unconstrained scenarios in which detection is desired and/or required by regulatory standards. Existing methods of predicting child presence based on age (e.g. direct age estimation from an image of an occupant or behavioral patterns in temporal sequences generated using a camera) can miss a detection, for example, in scenarios where visual occlusions are present (e.g., there is a blanket over an occupied child seat or a child's face, the view of a passenger's head is blocked by a car seat, a passenger's head pose is in an extreme position such as looking down or backwards). Some techniques use RADAR to supplement camera-based implementations to detect child presence by measuring parameters such as distance, movement, speed of movement, direction of movement, and/or angular offsets, but those techniques struggle to capture granular details of an individual's size and pose, which can be important in detecting age. Furthermore, RADAR sensors can struggle to distinguish activity within the vehicle from activity outside of - but within a close proximity to - the vehicle. Depending on the sensor layout, some of these issues may be present and/or more frequent in some regions in the cabin, but not others. As such, there is a need for improved child presence detection techniques that can reduce the risk of missed detections.

Whether for child presence detection or other tasks, implementing the logic to choose which sensor to use in a given scenario in the application layer comes with increased computational overhead, power consumption, and latency, whereas implementing the logic in the base drive stack limits OEM customizability. As such, there is a need for improved sensor selection and/or sensor fusion techniques for tasks such as driver and occupant monitoring tasks implemented in vehicle compute platforms or other ego-machine compute platforms.

### SUMMARY

Embodiments of the present disclosure relate to configurable sensor fusion, slot-dependent sensor fusion, and/or child presence detection for autonomous and semi-autonomous machines and applications.

Some embodiments apply occupant slot-dependent sensor selection, sensor fusion, and/or detection logic. Generally, any number of sensors may be positioned and/or dispersed in a variety of ways to observe any number of occupant slots (e.g., one or more seats, footwells, and/or other potential occupant positions), and some sensors may have a better view of some occupant slots (or may provide more reliable data in certain circumstances) than other sensors. As such, detection pipelines and/or state machines may be tailored to individual occupant slots (or groups of occupant slots), and tailored detection pipelines and/or state machines may be applied to detect and generate a unified representation of whether one or more conditions are present in a given occupant slot. Taking child presence detection as an example, size-based age estimation may be used for some occupant slots (e.g., those in a first row of vehicle seating), but not others (e.g., vehicle footwells). In some embodiments, different fusion logic may be applied for occupant slots in a first row of vehicle seating, a second row of vehicle seating, vehicle footwells, and/or other occupant slots. As such, the way in which sensor data and/or corresponding detections is combined may be tailored to a particular occupant slot.

In some embodiments, a sensor fusion algorithm in a base drive stack may be configured (e.g., by an OEM) using (e.g., slot-dependent) weights, thresholds, flags, values, and/or other parameters. Continuing with child presence detection (CPD) as an example, camera detection result(s) and RADAR detection result(s) may be combined using configurable (e.g., slot-dependent) weights to generate a fused or unified child (and/or adult) presence classification (e.g., a binary result, detection confidence). In some embodiments, logic that performs false positive and/or false negative suppression may be toggled (using a slot-dependent flag or value) and used to determine whether to overwrite or update a fused or unified child (and/or adult) presence classification, and the logic may use one or more configurable (e.g., slot-dependent) thresholds.

Although some embodiments involve configurable sensor fusion for child presence detection, other types of tasks may be additionally or alternatively be implemented using configurable sensor fusion to generate any number and type of corresponding unified detection results, such as hands-on-wheel detection, body size detection, abnormal pose detection, heart rate monitoring, and liveness detection, to name a few examples. More generally, a sensor fusion algorithm in a base control stack (e.g., a drive stack) of an ego-machine compute platform (e.g., a vehicle compute platform) may use configurable weights, thresholds, flags, values, and/or other parameters to customize whether and how data from multiple sensors or corresponding detection pipelines (e.g., raw sensor data, processed sensor data, computer vision or deep learning perception data) is combined. As such, a sensor fusion algorithm in a base control (e.g., drive) stack may be configured (e.g., by an OEM) to customize a (e.g., slot-dependent) sensor selection, sensor fusion, and/or detection task.

### BRIEF DESCRIPTION OF THE DRAWINGS

The present systems and methods for configurable sensor fusion, slot-dependent sensor fusion, and/or child presence detection for autonomous and semi-autonomous machines and applications are described in detail below with reference to the attached drawing figures, wherein:

FIG. 1 is a data flow diagram illustrating a data flow through an example ego-machine compute platform, in accordance with some embodiments of the present disclosure;

FIG. 2 illustrates an example state machine for fusing child presence detection results and which may be used in a first row of seating, in accordance with some embodiments of the present disclosure;

FIG. 3 an example state machine for fusing child presence detection results and which may be used in a first row of footwells, in accordance with some embodiments of the present disclosure;

FIGS. 4A and 4B illustrate an example state machine for fusing child presence detection results and which may be used in a second row of seating, in accordance with some embodiments of the present disclosure;

FIG. 5 illustrates an example state machine for fusing child presence detection results and which may be used in a second and third rows of footwells, in accordance with some embodiments of the present disclosure;

FIG. 6 illustrates an example state machine for fusing child presence detection results and which may be used in a third row of seating, in accordance with some embodiments of the present disclosure;

FIG. 7 illustrates some example child presence detection nodes which may be used for configurable sensor fusion, in accordance with some embodiments of the present disclosure;

FIG. 8 illustrates some possible outputs from an example RADAR child presence detection node, in accordance with some embodiments of the present disclosure;

FIG. 9 illustrates some possible outputs from an example camera child presence detection node, in accordance with some embodiments of the present disclosure;

FIG. 10A illustrates an example state machine which may be implemented by a camera child presence detection node for a first row of seating, in accordance with some embodiments of the present disclosure;

FIG. 10B illustrates an example state machine which may be implemented by a camera child presence detection node for a first row footwell, in accordance with some embodiments of the present disclosure;

FIG. 10C illustrates an example state machine which may be implemented by a camera child presence detection node for a second row of seating, in accordance with some embodiments of the present disclosure;

FIG. 11A illustrates an example state machine which may be used to fuse RADAR and camera detection results for occupant slot(s) in a first row of seating, in accordance with some embodiments of the present disclosure;

FIG. 11B illustrates an example state machine which may be implemented by a child seat overwrite node, in accordance with some embodiments of the present disclosure;

FIG. 12A illustrates an example state machine which may be implemented by a sensor fusion node for a first or second rows of seating, in accordance with some embodiments of the present disclosure;

FIG. 12B illustrates an example state machine which may be implemented by a false negative suppression node for child presence detection, in accordance with some embodiments of the present disclosure;

FIG. 12C illustrates an example state machine which may be implemented by a false positive suppression node for child presence detection, in accordance with some embodiments of the present disclosure;

FIG. 13 illustrates an example state machine which may be used to fuse RADAR and camera detection results for occupant slot(s) in a first row footwell, in accordance with some embodiments of the present disclosure;

FIG. 14 illustrates an example generalized state machine which may be which may be used to fuse RADAR and camera detection results for one or more occupant slots, in accordance with some embodiments of the present disclosure;

FIG. 15 is a flow diagram showing a method for child presence detection with slot-dependent logic, in accordance with some embodiments of the present disclosure;

FIG. 16 is a flow diagram showing a method for detecting whether one or more conditions are present using fusion logic with one or more configurable parameters, in accordance with some embodiments of the present disclosure;

FIG. 17 is a flow diagram showing a method for child presence detection using fusion logic with one or more configurable parameters, in accordance with some embodiments of the present disclosure;

FIG. 18A is an illustration of an example autonomous vehicle, in accordance with some embodiments of the present disclosure;

FIG. 18B is an example of camera locations and fields of view for the example autonomous vehicle of FIG. 18A, in accordance with some embodiments of the present disclosure;

FIG. 18C is a block diagram of an example system architecture for the example autonomous vehicle of FIG. 18A, in accordance with some embodiments of the present disclosure;

FIG. 18D is a system diagram for communication between cloud-based server(s) and the example autonomous vehicle of FIG. 18A, in accordance with some embodiments of the present disclosure;

FIG. 19 is a block diagram of an example computing device suitable for use in implementing some embodiments of the present disclosure; and

FIG. 20 is a block diagram of an example data center suitable for use in implementing some embodiments of the present disclosure.

### DETAILED DESCRIPTION

Systems and methods are disclosed related to configurable sensor fusion, slot-dependent sensor fusion, and/or child presence detection for autonomous and semi-autonomous machines and applications. Configurable sensor fusion may be used for detection tasks such as those involved in driver or occupant monitoring, and may be used by autonomous vehicles, semi-autonomous vehicles, robots, and/or other object or machine types. Taking child presence detection as an example, the present techniques may be used to detect the presence of child, detect the presence of an unattended child, determine age or size of a particular occupant, and/or take some responsive action (e.g., trigger an alarm, control temperature, unlock door(s), permit or disable airbag deployment, etc.) in systems with occupant monitoring (e.g., driver and/or passenger monitoring) and/or other types of systems.

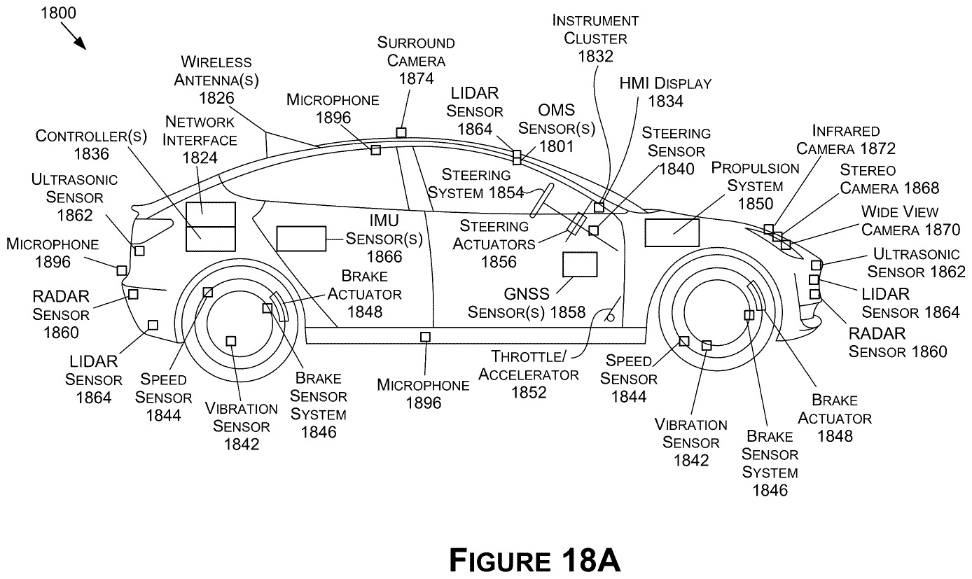

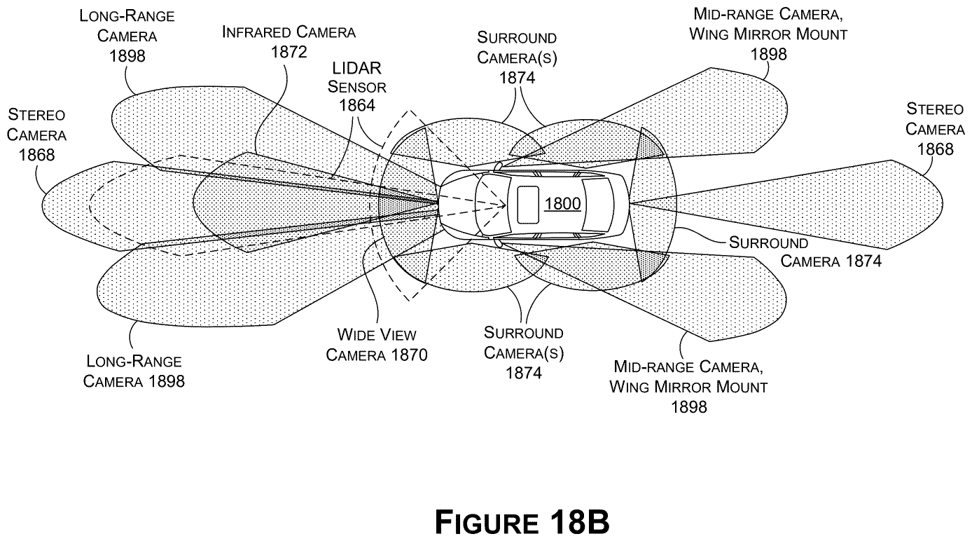

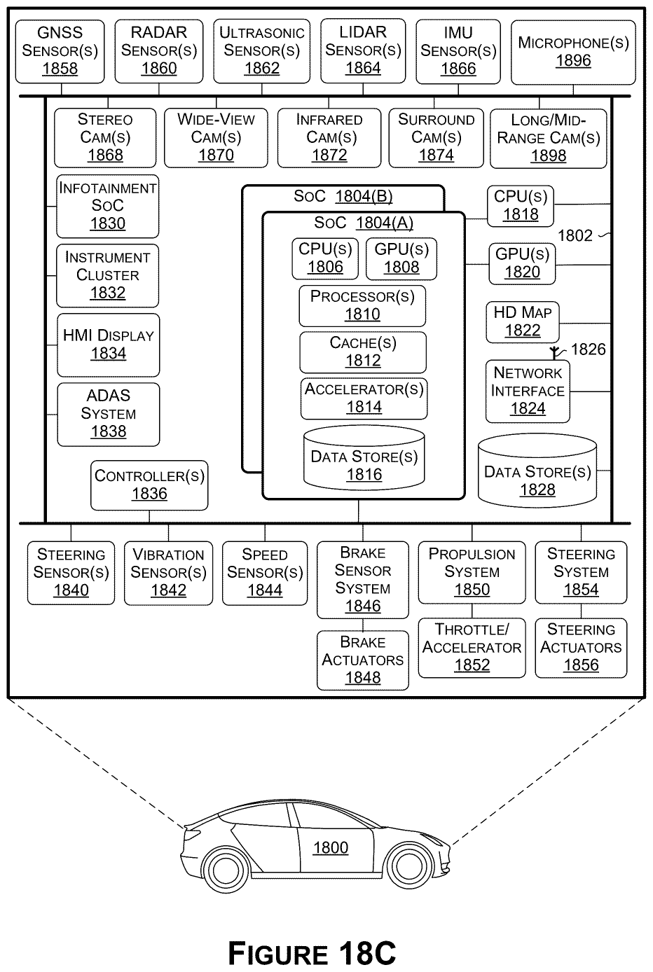

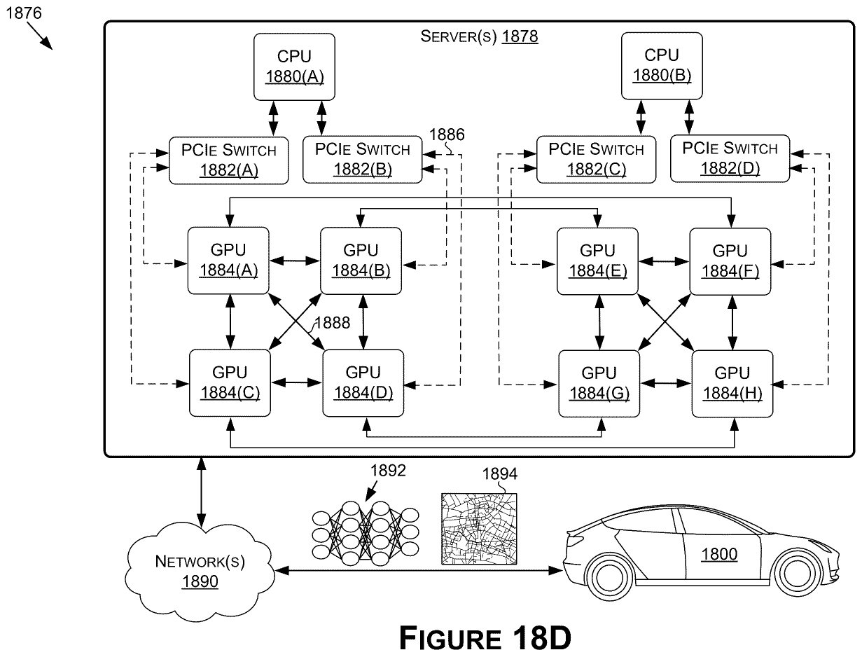

Although the present disclosure may be described with respect to an example autonomous or semi-autonomous vehicle or machine 1800 (alternatively referred to herein as “vehicle 1800” or “ego-machine 1800,” an example of which is described with respect to FIGS. 18A-18D), this is not intended to be limiting. For example, the systems and methods described herein may be used by, without limitation, non-autonomous vehicles or machines, semi-autonomous vehicles or machines (e.g., in one or more advanced driver assistance systems (ADAS)), autonomous vehicles or machines, piloted and un-piloted robots or robotic platforms, warehouse vehicles, off-road vehicles, vehicles coupled to one or more trailers, flying vessels, boats, shuttles, emergency response vehicles, motorcycles, electric or motorized bicycles, aircraft, construction vehicles, trains, underwater craft, remotely operated vehicles such as drones, and/or other vehicle types. In addition, although the present disclosure may be described with respect to child presence detection and/or configurable sensor fusion for autonomous driving, this is not intended to be limiting, and the systems and methods described herein may be used in augmented reality, virtual reality, mixed reality, robotics, security and surveillance, autonomous or semi-autonomous machine applications, and/or any other technology spaces where sensor fusion, presence detection, and/or age detection may be used.

In an example in-cabin or cockpit monitoring system such as a vehicle occupant monitoring system (OMS), one or more sensors (e.g., cameras, RGB sensors, infrared (IR) sensors, depth sensors such as RADAR sensors, etc.) may be positioned to observe one or more occupants within a cabin, cockpit, or other space. For example, an OMS may comprise a driver monitoring system (DMS), a system that monitors non-driver occupants, or a system that monitors driver occupant(s) and/or non-driver occupant(s). Any number of sensors may be positioned and/or dispersed in a variety of ways to observe any number of occupant slots (e.g., one or more seats, footwells, and/or other potential occupant positions). Taking an example sensor layout in an example vehicle with two rows of seats, one or more cameras may be positioned in the front of the vehicle facing toward the back (e.g., positioned in or around the rear view mirror and oriented with a field of view of the faces of occupants sitting in the front and back rows), overhead and facing down (e.g., with a field of view of the faces of occupants sitting in the back row), and/or otherwise. Additionally or alternatively, one or more RADAR sensors may be positioned (e.g., toward the back of the vehicle, such as above the rear window) and oriented (e.g., facing forward) with a field of view of occupants sitting in the front and back rows. These are just a few examples of possible sensor layouts, and other layouts of these and/or other sensors within any suitable scene may be implemented within the scope of the present disclosure.

Generally, some sensors may have a better view of some occupant slots than others. Taking an example sensor layout in which an OMS camera is positioned in the front of the vehicle facing toward the back, visibility of occupants in the second row of seating may be limited (e.g., to the chest and below). As a result, child presence detection techniques that rely on size-based age estimation from images may not work on occupants in the second row using that camera position or sensor layout. Similarly, the vehicle footwells may be occluded from the OMS camera(s), so it may not be possible to perform size-based age estimation for child presence detection on certain vehicle footwells using that camera position or sensor layout.

As such, some embodiments apply occupant slot-dependent sensor selection, sensor fusion, and/or detection logic. Continuing with child presence detection as an example, in some embodiments (e.g., that use a sensor layout in which one or more cameras have sufficient visibility for size-based age estimation of occupants in one or more occupant slots, such as those in a first row of vehicle seating), example slot-dependent detection logic may determine whether 1) camera presence or occupancy detection or 2) RADAR blockage detection classifies a view of the slot as clear and RADAR liveness detection classifies each applicable slot as occupied. If so, child seat detection may be performed (or its results looked up). If camera presence or occupancy detection classifies a slot as occupied, face and size-based age estimation may be performed, and/or the results of child seat detection, face-based age estimation, and size-based age estimation may be combined (e.g., using a weighted combination of predictions and confidences) to determine whether the slot is occupied by a child or adult.

In some embodiments (e.g., that use a sensor layout in which one or more cameras do not have sufficient visibility for size-based age estimation of occupants in one or more occupant slots, such as those in a second row of vehicle seating), example slot-dependent detection logic may determine whether RADAR blockage detection classifies a view of the slot as clear, RADAR liveness detection classifies a live occupant, and RADAR pet detection classifies a human-like signature in each applicable slot, and if so, RADAR occupant classification may be performed (or its results looked up). If the RADAR classification identifies a child in the slot, face-based age estimation may be performed and/or its results may be used to reject false positives. If the RADAR classification identifies an adult in the slot, face-based age estimation may be performed and/or its results may be used to reject false negatives. If the RADAR classification is unknown, child seat detection may be performed and/or its results may be used to confirm the presence of a child. If child seat detection does not identify the presence of a child seat, face-based age estimation may be performed and/or its results may be used to identify the presence of an adult occupant.

In some embodiments (e.g., that use a sensor layout in which one or more cameras do not have sufficient visibility for size-based age estimation of occupants in one or more occupant slots, such as those in a front row of vehicle footwells), example slot-dependent detection logic may determine whether RADAR blockage detection classifies a view of the slot as clear, RADAR liveness detection classifies a live occupant, and RADAR pet detection classifies a human-like signature in each applicable slot. In some embodiments (e.g., for one or more occupant slots in vehicle footwells other than in the first row), example slot-dependent detection logic may determine whether RADAR blockage detection classifies a view of the slot as clear, RADAR liveness detection classifies a live occupant, RADAR pet detection classifies a human-like signature, and RADAR age estimation classifies the human occupant as a child in each applicable slot. These are meant simply as examples, and variations may be implemented within the scope of the present disclosure.

In some embodiments, a sensor fusion algorithm in a base drive stack may be configured (e.g., by an OEM) using (e.g., slot-dependent) weights, thresholds, flags, values, and/or other parameters (e.g., via a vehicle application layer and one or more APIs). Continuing with child presence detection (CPD) as an example, camera detection result(s) (e.g., of presence or occupancy detection, child presence classification, age estimation) and RADAR detection result(s) (e.g., of RADAR blockage detection, liveness detection, pet detection, child presence classification) may be combined using configurable (e.g., slot-dependent) weights to generate a fused or unified child (and/or adult) presence classification (e.g., a binary result, detection confidence). In some embodiments, a child seat overwrite node or logic may be toggled (using a slot-dependent flag or value) and used to overwrite a false negative (a missed detection of a present child) when RADAR liveness detects a human-like signature (e.g., with a configurable threshold confidence) and camera presence or occupancy detection classifies the presence of child seat (e.g., with a configurable threshold confidence). Additionally or alternatively, a false negative suppression node or logic may be toggled (using a slot-dependent flag or value) and used to overwrite a false negative when a RADAR child presence classification is unknown or identifies an adult, camera presence or occupancy detection classifies the presence of an occupant (e.g., with a configurable threshold confidence), and child seat detection classifies the presence of a child seat (e.g., with a configurable threshold confidence). In some embodiments, a false positive suppression node or logic may be toggled (using a slot-dependent flag or value) and used to overwrite a false positive (an incorrectly detected child) when a RADAR child presence classification identifies the presence of a child, camera presence or occupancy detection classifies the presence of an occupant (e.g., with a configurable threshold confidence), and camera age classification classifies the occupant as an adult (e.g., above 18 years old) (e.g., with a configurable threshold confidence).

Although some embodiments involve configurable sensor fusion for child presence detection, other types of tasks may be implemented using configurable sensor fusion, such as hands-on-wheel detection, body size detection, abnormal pose detection, heart rate monitoring, and liveness detection, to name a few examples. More generally, a sensor fusion algorithm in a base control stack (e.g., a drive stack) of an ego-machine compute platform (e.g., a vehicle compute platform) may use configurable weights, thresholds, flags, values, and/or other parameters to customize whether and how data from multiple sensors or corresponding detection pipelines (e.g., raw sensor data, processed sensor data, computer vision or deep learning perception data) is combined. In some embodiments, a sensor fusion algorithm in a base control stack may directly combine, weight, and/or apply threshold(s) to results from corresponding detection pipelines (e.g., for body size detection and/or child presence detection, combining the detection confidence from a first body size classification based on a weight sensor with the detection confidence from a second body size classification based on camera perception). In some embodiments, configurable sensor fusion may be used to overcome limitations of certain sensors for certain tasks and/or increase the reliability of the fused results (e.g., by suppressing false positives and/or false negatives). Taking hands-on-wheel detection as an example, a capacitive touch sensor in a steering wheel is typically sensitive to temperature and moisture, so extreme temperatures or wet hands may lead to missed detections. Camera perception may be used to suppress these false negatives, but may miss detections itself (e.g., in poor lighting). As such, a sensor fusion algorithm in a base control stack may directly combine, weight, and/or apply threshold(s) results from corresponding detection pipelines (e.g., predicted confidences generated based on a capacitive touch sensor, a torque sensor, camera perception, etc.). These are just a few examples, and others may be implemented within the scope of the present disclosure.

As such, a sensor fusion algorithm in a base control (e.g., drive) stack may be configured (e.g., by an OEM) to customize a (e.g., slot-dependent) sensor selection, sensor fusion, and/or detection task. By providing the base control stack with the configurable parameters (e.g., and storing them locally, such as in one or more lookup tables), the applicable (e.g., sensor selection, sensor fusion, detection task) functionality of the base control stack may be customized without using custom logic in the (e.g., vehicle) application layer. As such, the base control stack and compute platform may execute the applicable functionality without the computational overhead, increased power consumption, and latency that would otherwise occur from integrating custom OEM logic through an API(s) in the application layer. Furthermore, the weights, thresholds, flags, values, and/or other parameters may be updated after deployment (e.g., at a service center, as part of a software update, in response to upgrading or replacing one or more sensors, etc.), further enhancing customizability.

As such, the present techniques may be used to increase the accuracy and performance of driver or occupant monitoring tasks and/or other tasks that use data from multiple sensors.

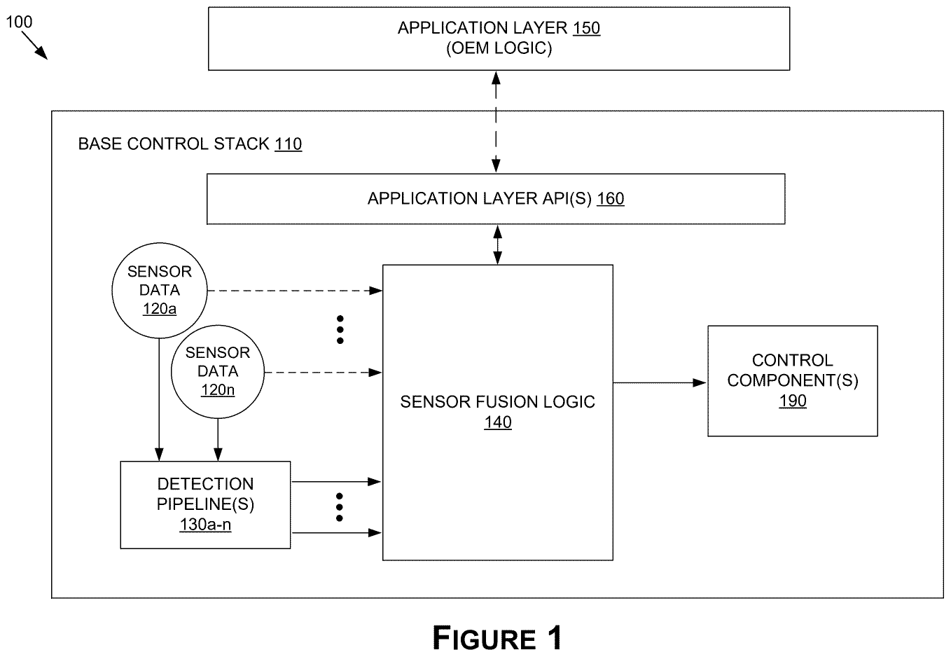

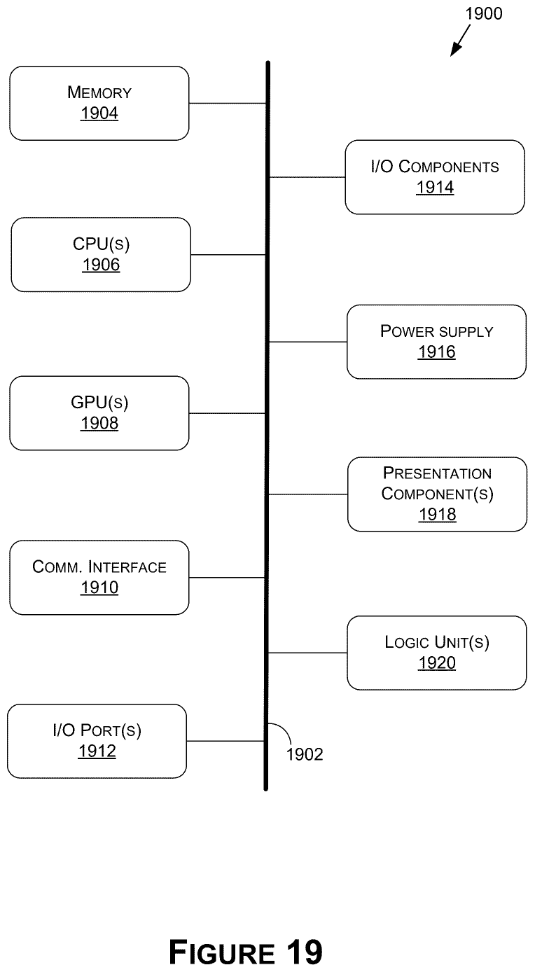

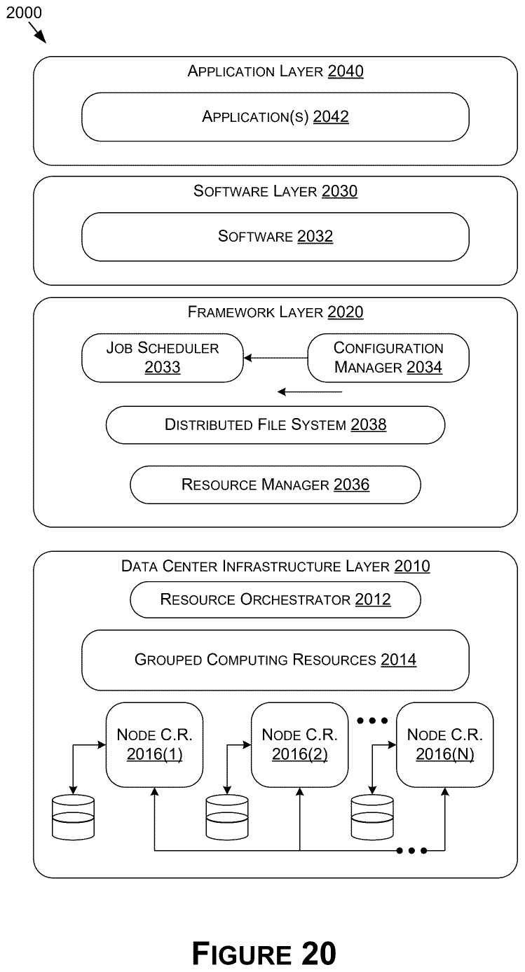

With reference to FIG. 1, FIG. 1 is an example ego-machine compute platform 100, in accordance with some embodiments of the present disclosure. It should be understood that this and other arrangements described herein are set forth only as examples. Other arrangements and elements (e.g., machines, interfaces, functions, orders, groupings of functions, etc.) may be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by entities may be carried out by hardware, firmware, and/or software. For instance, various functions may be carried out by a processor executing instructions stored in memory. In some embodiments, the systems, methods, and processes described herein may be executed using similar components, features, and/or functionalities to those of example autonomous vehicle 1800 of FIGS. 18A-18D, example computing device 1900 of FIG. 19, and/or example data center 2000 of FIG. 20.

At a high level, the ego-machine compute platform 100 may be deployed on an ego-machine (e.g., the autonomous vehicle 1800 of FIGS. 18A-18D) and may serve as a central processing hub, executing software (e.g., in the base control stack 110 and/or application layer 150) that implements real-time perception, detection, and/or control of the ego-machine. There are different types of ego-machine compute platforms designed for different applications, such as automotive (e.g., the NVIDIA DRIVE AGX™ platform), robotics (e.g., the NVIDIA Jetson™ platform), and aerial (e.g., Qualcomm® Flight RB5™). The ego-machine compute platform 100 may run in real-time on a system-on-chip of an ego-machine (e.g., an automotive SoC such as NVIDIA's DRIVE Orin™ SoC, which may serve as a central computer for in-vehicle computing and may correspond to the SoC(s) 1804 of FIG. 18C). Other types of SoCs which may be used in compute platforms for robots or ego-machines include AI-optimized SoCs (e.g., NVIDIA Jetson Series), robotics SoCs (e.g., for robotics applications such as drones, service robots, or industrial robots), industrial SoCs (e.g., for industrial robots or automation systems for factory automation, robotics, or control systems), drone and unmanned aerial vehicle (UAV) SoCs (e.g., for handling flight control, navigation, and/or real-time video processing), and/or others.

In the example shown in FIG. 1, the ego-machine compute platform 100 includes a base control stack 110 (e.g., a base drive stack) and an application layer 150 (e.g., vehicle application layer). The base control stack 110 that comes with the ego-machine compute platform 100 may manage various functions of the ego-machine based on processing sensor data 120a-n through detection pipeline(s) 130a-n, using sensor fusion logic 140 to create a unified representation of the environment, and using control component(s) 190 to take one or more responsive actions (e.g., issuing navigation control actions such as steering, acceleration, and breaking; issuing audible or visual alerts; adjusting in-vehicle infotainment settings; activating safety systems like adaptive cruise control or lane-keeping assistance; controlling in-cabin temperature; unlocking door(s); permitting or disabling airbag deployment; etc.). Generally, the sensor(s), the sensor data 120a-n, the detection pipeline(s) 130a-n, the sensor fusion logic 140, the control component(s) 190, and/or the responsive action may depend on the detection task and/or the implementation.

Software in the application layer 150 may be used (e.g., by a manufacturer or OEM) to customize and fine-tune how the ego-machine behaves. While the base control stack 110 may provide standardized functionality, the application layer 150 may enable OEMs to introduce proprietary algorithms or unique navigation behaviors. Depending on the implementation, the application layer 150 may introduce custom decision-making logic that modifies how the sensor fusion logic 140 interprets the sensor data 120a-n and/or corresponding detections from the detection pipeline(s) 130a-n. For example, the base control stack 110 may provide one or more interfaces such as application layer API(s) 160 that allow OEMs to customize or override certain functionalities within the base control stack 110 (e.g., within the sensor fusion logic 140). Depending on the embodiment, the application layer API(s) 160 may enable the application layer 150 to interact with the underlying sensor fusion logic 140 and/or detection pipeline(s) 130a-n, allowing custom logic to refine or adjust the way the sensor data 120a-n is processed and/or interpreted. In some embodiments, instead of (or in addition to) using custom logic in the application layer 150 (e.g., to refine or adjust the way the sensor data 120a-n is processed and/or interpreted by the sensor fusion logic 140), the sensor fusion logic 140 may be customized by setting one or more (e.g., slot-dependent) weights, thresholds, flags, values, and/or other parameters in the base control stack 110 (e.g., via the application layer API(s) 160, middleware API(s), developer API(s), diagnostic API(s), etc.).

Continuing with a high-level overview, one or more sensors may be used to generate sensor data 120a-n representing an interior or exterior space. The sensor(s) may include any number and/or any type of sensor, such as, without limitation, one or more cameras, LiDAR sensors, RADAR sensors, weight sensors, capacitive touch sensors, torque sensor, and/or other sensor types such as those described below with respect to the autonomous vehicle 1800 of FIGS. 18A-18D. Taking a perception task such as a driver monitoring systems (DMS) or occupant monitoring systems (OMS) task an example, the sensor(s) may be positioned to perceive one or more humans or other subjects (e.g., objects left behind) in an interior or exterior space or other environment in which one or more humans or other subjects may be present (e.g., seating, footwells, etc.), and may be used to generate frames of corresponding sensor data 120a-n (e.g., image data, RADAR data, etc.) at any suitable frame rate. In some embodiments, the sensor(s) may include one or more RADAR sensors used to generate RADAR data (e.g., a serialized or encoded point cloud, a point cloud projected onto a 2D image such as a range image or a top-down image, with reflection characteristics of detected points populated in corresponding channels, accumulated over some duration or number of spins, etc.). Depending on the type of sensor and/or implementation, reflection characteristics may include bearing, azimuth, elevation, range (e.g., time of beam flight), intensity, Doppler velocity, RADAR cross section (RCS), reflectivity, SNR, and/or the like.

In some embodiments, the sensor(s) may include one or more sensors of an ego-machine (e.g., an OMS or DMS camera such as the OMS sensor(s) 1801 of the vehicle 1800; one or more exterior cameras such as the stereo camera(s) 1868, wide-view camera(s) 1870 (e.g., fisheye cameras), infrared camera(s) 1872, surround camera(s) 1874, and/or long-range and/or mid-range camera(s) 1898 of the vehicle 1800; one or more RADAR sensors such as the RADAR sensor(s) 1860 of the vehicle 1800; etc.), and the sensor(s) may be used to generate frames of sensor data 120a that represent an environment being monitored (e.g., an environment outside an ego-machine, an interior space), an operator or occupant of an ego-machine, or some other monitored subject or space. In some embodiments, sensor data from different sensors and/or different types of sensors may be temporally aligned (e.g., using one or more configurable offsets, which may corresponding to an anticipated latency for each sensor or sensor type), to identify and select associated sensor data 120a-n (e.g., one or more images and corresponding RADAR data) representing substantially the same time slice.

Taking a monitoring system such as a vehicle OMS as an example, one or more optical sensors and/or RADAR sensors may be positioned to perceive a scene within a cabin, cockpit, or other interior or exterior space. An OMS may comprise a DMS, a system that monitors non-driver occupants, or a system that monitors driver (operator) occupant(s) and/or non-driver occupant(s). OMSs often rely on perception from multiple optical sensors (e.g., RGB sensors, infrared IR sensors, depth sensors, cameras, etc.) positioned at various locations throughout a vehicle interior. Vehicle manufacturers tend to vary the number of OMS cameras from model to model and depending on the trim level. Base models usually have one camera facing the driver (e.g., positioned within a steering column, vehicle pillar, or infotainment console). Higher trim levels may include any number of additional cameras (e.g., one in the steering column facing the driver, one in the rear review mirror facing the driver or the cabin, one in a vehicle pillar facing a particular row of seating, one above a row of headrests facing forward for child detection, etc.). Generally, occupant and/or driver monitoring systems may include any number of cameras (e.g., 4 DMS cameras and 16 OMS cameras) positioned throughout a vehicle interior. RADAR sensor(s) may be positioned to monitor occupants and/or the cabin environment (e.g., installed in an overhead console monitoring the entire cabin, integrated into the dashboard or a front or rear windshield facing towards the cabin, etc.). These are just a few examples of possible sensor layouts, and other sensor layouts within any suitable interior scene (e.g., supermarket aisle, hospital operating room, retail store, office space, manufacturing facility, warehouse facility, etc.) or exterior scene (e.g., city street, construction site, agricultural field, public transportation hub, urban environment, etc.) may be implemented within the scope of the present disclosure.

Depending on the detection task and/or the implementation, different types of sensor(s), sensor data 120a-n, detection pipeline(s) 130a-n, and/or sensor fusion logic 140 may be involved. In some embodiments, any known detection pipeline may be used to detect a condition from corresponding sensor data and generate a representation of the detected condition.

Taking a DMS task such as driver drowsiness or distraction detection as an example, the sensor(s) may include camera(s), steering wheel (e.g., torque) sensors, heart rate sensors, and/or accelerometers; the detection pipeline(s) 130a-n may use any known technique (e.g., to evaluate camera image(s) and) detect and generate a representation of detected facial expressions and/or eye movements such as eye blinking, and the sensor fusion logic 140 may use (e.g., configurable, preset) parameters to select, combine, weight, and/or apply threshold(s) to (e.g., binary) signals or other representations of (prediction confidence in) detected facial expressions, eye blink rate, heart rate, and/or unusual vehicle acceleration such as swaying or inconsistent lane keeping to generate a unified representation of detected drowsiness or distraction (e.g., a unified detection confidence, a binary result).

Taking hands-on-wheel detection as an example, the sensor data 120a-n may include electrical capacitance measured using capacitive sensor(s) embedded in the steering wheel, force applied by the driver's grip measured using pressure sensor(s), image(s) of the driver's hands and their position relative to the wheel, torque exerted by the driver on the wheel measured using torque sensor(s), and/or others. The detection pipeline(s) 130a-n may use any known technique (e.g., to evaluate camera image(s) and) detect and generate a representation of whether the driver's hands are detected on the steering wheel, and the sensor fusion logic 140 may use (e.g., configurable, preset) parameters to select, combine, weight, and/or apply threshold(s) to signals corresponding to the sensor data 120a-n and/or the representation of whether the driver's hands were detected on the steering wheel by a corresponding detection pipeline(s) 130a-n (e.g., detection confidence(s)) to generate a unified representation of whether the driver's hands were detected on the steering wheel (e.g., a unified detection confidence, a binary result).

Taking body size detection as an example, the sensor data 120a-n may include weight measured using weight sensor(s) embedded in the seat cushion; the force or force distribution across the seat measured using pressure sensor(s) embedded in the seat cushion; depth or depth map(s) measured using ultrasonic sensor(s), infrared sensor(s), or RADAR sensor(s); and/or others. The detection pipeline(s) 130a-n may use any known technique to estimate 3D pose, body size, and/or body shape (e.g., based on force or pressure distribution maps, detected depth or depth maps, images, etc.), and the sensor fusion logic 140 may use (e.g., configurable, preset) parameters to select, combine, weight, and/or apply threshold(s) to signals corresponding to the sensor data 120a-n (e.g., force or pressure distribution, weight) and/or different estimates of 3D pose, body size, and/or body shape to generate a unified representation of estimated body size.

Taking abnormal pose detection as an example, the sensor data 120a-n may include images or video of an occupant or occupant slot; the force or force distribution across the seat measured using pressure sensor(s) embedded in the seat cushion; depth or depth map(s) measured using ultrasonic sensor(s), infrared sensor(s), or RADAR sensor(s); and/or others. The detection pipeline(s) 130a-n may use any known technique to estimate the 3D pose of an occupant (e.g., based on image data, depth, etc.) and compare the occupant's pose with standard or expected poses, to compare a measured force distribution with baseline posture distributions, and/or otherwise, and the sensor fusion logic 140 may use (e.g., configurable, preset) parameters to select, combine, weight, and/or apply threshold(s) to signals corresponding to the sensor data 120a-n (e.g., force or pressure distribution, weight) and/or a representation of whether an abnormal pose was detected by a corresponding detection pipeline(s) 130a-n (e.g., detection confidence(s)) to generate a unified representation of a detected abnormal pose (e.g., a unified confidence, a binary result).

Taking heart rate monitoring as an example, the sensor data 120a-n may include voltage differences across the body representing the electrical activity of the heart measured using electrocardiogram (ECG) sensor(s) embedded in the seat or steering wheel, thermal radiation or skin temperature measured using infrared sensor(s), light absorption variations representing changes in blood flow (which correlates with heart rate) measured by photoplethysmography (PPG) sensor(s) integrated into the steering wheel or seat, detected phase or Doppler shift in reflected RADAR signals (e.g., which correspond to distance and velocity changes due to micro-movements of the chest wall caused by the heart beating), and/or others. As such, the detection pipeline(s) 130a-n may use any known technique to estimate heart rate from the sensor data 120a-n (e.g., extracting heart rate from the ECG waveform, measured light absorption variations, detected phase or Doppler shift in reflected RADAR signals, etc.), and the sensor fusion logic 140 may use (e.g., configurable, preset) parameters to select, combine, weight, and/or apply threshold(s) to signals corresponding to the sensor data 120a-n (e.g., changes in skin temperature) and/or different estimates of heart rate to generate a unified representation of estimated heart rate.

Taking liveness detection (e.g., determining whether an occupant or driver is physically present and responsive) as an example, the sensor data 120a-n may include detected phase or Doppler shift in reflected RADAR signals (e.g., which indicate micro-movements of the chest or body such as breathing and heartbeats), thermal radiation or skin temperature measured using infrared sensor(s), the force or force distribution across the seat measured using pressure sensor(s) embedded in the seat cushion; images or video of an occupant or occupant slot; and/or others. The detection pipeline(s) 130a-n may use any known technique to detect micro-movements in the chest or body from phase or Doppler shift in reflected RADAR signals; detect shifts in posture or movement from changes in force or pressure distribution in the seat; perform facial detection and/or facial or eye tracking based on image, video, and/or thermal data; and/or otherwise. As such, the sensor fusion logic 140 may use (e.g., configurable, preset) parameters to select, combine, weight, and/or apply threshold(s) to signals corresponding to the sensor data 120a-n (e.g., force or pressure distribution, skin temperature) and/or a representation of whether a live occupant was detected by a corresponding detection pipeline(s) 130a-n (e.g., detection confidence(s)) to generate a unified representation of whether a live occupant was detected (e.g., a unified confidence, a binary result).

Taking child presence detection as an example, the sensor data 120a-n may include RADAR data (e.g., amplitude, SNR, reflection time-of-flight (ToF), phase shift, Doppler shift, frequency response, etc.) measured using RADAR sensor(s), reflection ToF measured using ultrasonic sensor(s), images or videos of an occupant or occupant slot, and/or otherwise. in some embodiments, the detection pipeline(s) 130a-n may use any known technique (or other techniques described herein) to detect RADAR blockage, to detect the presence of a live occupant in an occupant slot (e.g., RADAR liveness detection, applying face or person detection to an image), to classify a detected occupant in an occupant slot as an adult or child (e.g., based on RADAR data, based on image data), to detect the presence of a human or pet in an occupant slot (e.g., based on RADAR data, based on image data), to detect a child seat (e.g., from an image of an occupant slot), to estimate or classify the age of an occupant (e.g., face-based age estimation, size-based age estimation), and/or otherwise, and the sensor fusion logic 140 may use one or more (e.g., slot-dependent) state machines and/or (e.g., configurable, preset) parameters to select, combine, weight, and/or apply threshold(s) to (e.g., binary) signals or other representations of detection results (e.g., which may be representative of predicted presence) generated by a corresponding detection pipeline(s) 130a-n (e.g., detection confidence(s)) to generate a unified representation of whether a child (or adult) was detected in an occupant slot (e.g., a unified confidence, a binary result). These are meant simply as example tasks, corresponding sensor data 120a-n, and possible detection pipeline(s) 130a-n, and others are contemplated within the scope of the present disclosure.

In some embodiments, the sensor fusion logic 140 (and/or the application layer 150) may implement one or more state machines to select, combine, weight, and/or apply threshold(s) to signals corresponding to the sensor data 120a-n, one or more outputs of the detection pipeline(s) 130a-n (e.g., derived values, detection confidence(s) indicating a likelihood that one or more conditions were detected, etc.) to generate a unified representation of whether one or more conditions are detected (e.g., a unified confidence, a binary result). Depending on the task and/or the implementation, the one or more state machines may be tailored or specific to one or more occupant slots (e.g., vehicle seats, rows of seating, footwells, rows of footwells, etc.). For example, corresponding signals may be generated for each supported occupant slot, and the sensor fusion logic 140 (and/or the application layer 150) may look up and execute a corresponding state machine(s) for a corresponding occupant slot.

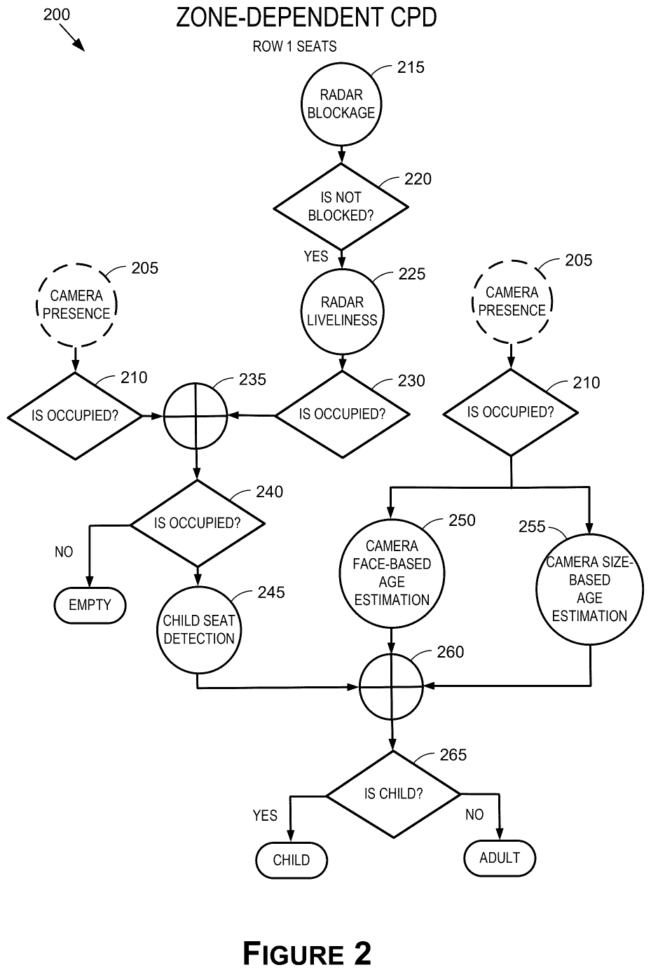

Taking child presence detection as an example, FIGS. 2-6 illustrate some example (e.g., slot-dependent) state machines for child presence detection which may be implemented in the sensor fusion logic 140 (and/or the application layer 150) for one or more occupant slots, in accordance with some embodiments of the present disclosure. The following examples involve various detection results (e.g., which may be generated by corresponding detection pipeline(s) 130a-n of FIG. 1 and which may be represented in any suitable manner), including results of RADAR blockage detection (e.g., a representation of whether blockage of a sensor RADAR was detected from corresponding RADAR data using any known technique), RADAR liveness detection (e.g., a representation of whether a live occupant was detected in an occupant slot from corresponding RADAR data using any known technique), RADAR pet detection (e.g., a representation of whether a human-like signature was detected in an occupant slot from corresponding RADAR data using any known pet detection technique), RADAR child presence or CPD classification (e.g., a representation of whether a child or adult was detected in an occupant slot from corresponding RADAR data using any known technique), RADAR age estimation (e.g., a representation of an estimated age or age range of an occupant in an occupant slot and/or a representation of whether the estimated age or age range falls within a corresponding range for an adult or child, estimated from corresponding RADAR data using any known technique, such as classification and/or regression), camera presence or occupancy detection (e.g., a representation of whether a person was detected in an occupant slot from a corresponding portion of an image(s) using any known technique, such as using a multi-class detector that segments and/or classifies regions of an image(s) as occupied by a person, occupied by a child seat, or empty and mapping classifications of region(s) of the image(s) to corresponding occupant slot(s)), camera child presence or CPD classification (e.g., a representation of whether a child or adult was detected in an occupant slot from a corresponding portion of an image(s) using any known technique), child seat detection (e.g., a representation of whether a child seat was detected in an occupant slot from a corresponding portion of an image(s) using any known technique, such as using a multi-class detector that segments and/or classifies regions of an image(s) as occupied by a person, occupied by a child seat, or empty and mapping classifications of region(s) of the image(s) to corresponding occupant slot), camera face-based age estimation (e.g., a representation of an estimated age or age range of an occupant in an occupant slot and/or a representation of whether the estimated age or age range falls within a corresponding range for an adult or child, estimated from an image(s) of the occupant's face using any known technique, such as classification and/or regression), and camera size-based age estimation (e.g., a representation of an estimated age or age range of an occupant in an occupant slot and/or a representation of whether the estimated age or age range falls within a corresponding range for an adult or child, estimated from an image(s) of the occupant's body using any known technique, such as based on detected limb length). These are meant simply as examples, and variations may be implemented within the scope of the present disclosure.

FIG. 2 illustrates an example state machine 200 which may be used to fuse one or more child presence detection results for one or more occupant slots such as those in a first row of seating. Taking an occupant slot such as the driver's seat as an example, at step 205, the state machine 200 may look up (or trigger generation of) a camera presence or occupancy detection result for the driver's seat (e.g., a detection confidence, a binary result), and at step 210, the state machine 200 may evaluate whether the camera presence or occupancy detection indicates the occupant slot is occupied (e.g., a positive detection with more than a threshold confidence). At step 215, the state machine 200 may look up (or trigger generation of) a RADAR blockage detection result (e.g., a detection confidence, a binary result), and at step 220, the state machine 200 may evaluate whether the RADAR blockage detection result indicates that the RADAR sensor is not blocked (e.g., classified as not blocked with more than a threshold confidence). If the state machine 200 determines the RADAR sensor is not blocked, at step 225, the state machine 200 may look up (or trigger generation of) a RADAR liveness detection result (e.g., a detection confidence, a binary result), and at step 230, the state machine 200 may evaluate whether the RADAR liveness detection indicates the occupant slot is occupied by a live occupant (e.g., a positive detection with more than a threshold confidence). At step 235, the state machine 200 may combine the results of steps 210 and 230 in any suitable manner to generate a unified representation of - or otherwise determine - whether the occupant slot is occupied. For example, the state machine 200 may act as an OR gate and determine whether the camera presence or occupancy detection indicates the slot is occupied or the RADAR liveness detection indicates the occupant slot is occupied by a live occupant. In some embodiments, the state machine 200 may generate a weighted combination of detection confidences and determine whether the resulting unified detection confidence is at least a threshold confidence. If the state machine 200 determines that the occupant slot is not occupied (e.g., there is less than a threshold confidence that the slot is occupied), the state machine 200 may classify the slot as empty (e.g., and generate a corresponding representation). Otherwise, the state machine 200 may advance to step 245 and look up (or trigger generation of) a child seat detection result (e.g., a detection confidence, a binary result).

If the state machine 200 determined that the camera presence or occupancy detection indicates the occupant slot is occupied (e.g., a positive detection with more than a threshold confidence) at step 210, it may advance to steps 250 and/or 255, looking up (or triggering generation of) camera face-based age estimation and/or camera size-based age estimation results. As such, at step 260, the state machine 200 may combine the child seat detection result from step 245 and the camera face-based age estimation and/or camera size-based age estimation results from steps 250 and/or 255 respectively in any suitable manner to generate a unified representation of - or otherwise determine - whether the occupant slot is occupied by a child. For example, the state machine 200 may generate a weighted combination of detection confidences, and at step 265, determine whether the resulting unified detection confidence indicates that the occupant slot is occupied by a child (e.g., with at least a threshold confidence). If so, the state machine 200 may classify the slot as occupied by a child (e.g., and generate a corresponding representation). Otherwise, the state machine 200 may classify the slot as occupied by an adult (e.g., and generate a corresponding representation).

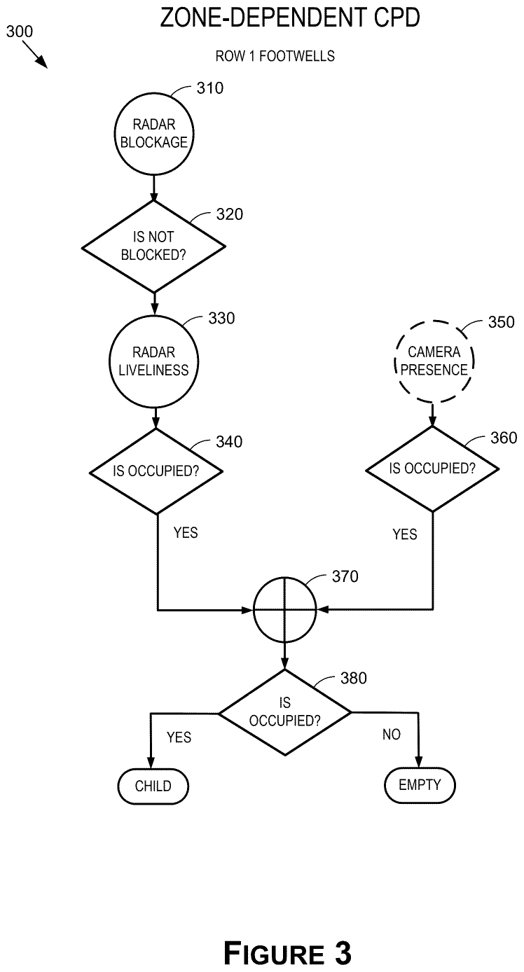

FIG. 3 illustrates an example state machine 300 which may be used to fuse one or more child presence detection results for one or more occupant slots such as those in a first row of footwells. At step 315, the state machine 300 may look up (or trigger generation of) a RADAR blockage detection result (e.g., a detection confidence, a binary result), and at step 330, the state machine 300 may evaluate whether the RADAR blockage detection result indicates that the RADAR sensor is not blocked (e.g., classified as not blocked with more than a threshold confidence). If the state machine 300 determines the RADAR sensor is not blocked, and taking an occupant slot such as the front row passenger seat footwell as an example, at step 330, the state machine 300 may look up (or trigger generation of) a RADAR liveness detection result for that slot (e.g., a detection confidence, a binary result), and at step 340, the state machine 300 may evaluate whether the RADAR liveness detection indicates the occupant slot is occupied by a live occupant (e.g., a positive detection with more than a threshold confidence). At step 350, the state machine 300 may look up (or trigger generation of) a camera presence or occupancy detection result for the occupant slot (e.g., a detection confidence, a binary result), and at step 360, the state machine 300 may evaluate whether the camera presence or occupancy detection indicates the occupant slot is occupied (e.g., a positive detection with more than a threshold confidence).

At step 370, the state machine 300 may combine the results of steps 340 and 360 in any suitable manner to generate a unified representation of - or otherwise determine - whether the occupant slot is occupied. For example, the state machine 300 may act as an OR gate and determine whether the camera presence or occupancy detection indicates the slot is occupied or the RADAR liveness detection indicates the occupant slot is occupied by a live occupant. In some embodiments, the state machine 300 may generate a unified detection confidence as a weighted combination of detection confidences. As such, at step 380, the state machine 300 may determine whether the slot is occupied (e.g., whether either of the RADAR or camera detections indicated the slot was occupied, whether the unified detection confidence is at least a threshold confidence). If so, the state machine 300 may classify the slot as occupied by a child (e.g., and generate a corresponding representation). Otherwise, the state machine 300 may classify the slot as empty (e.g., and generate a corresponding representation).

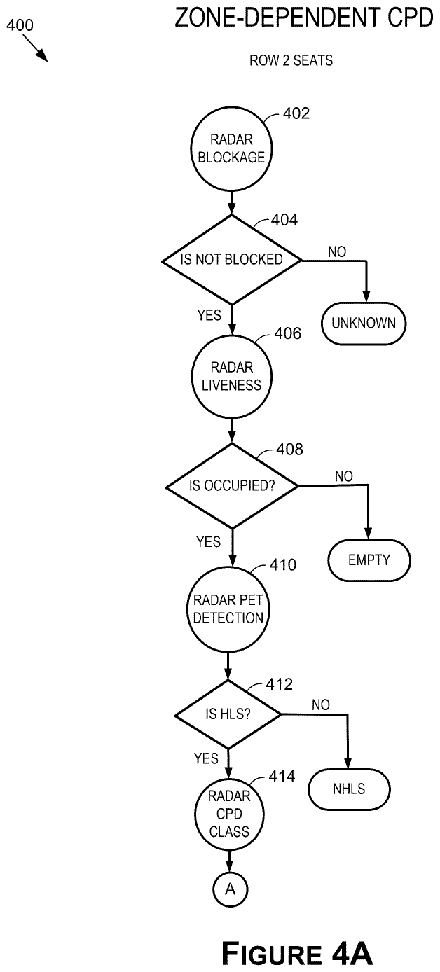

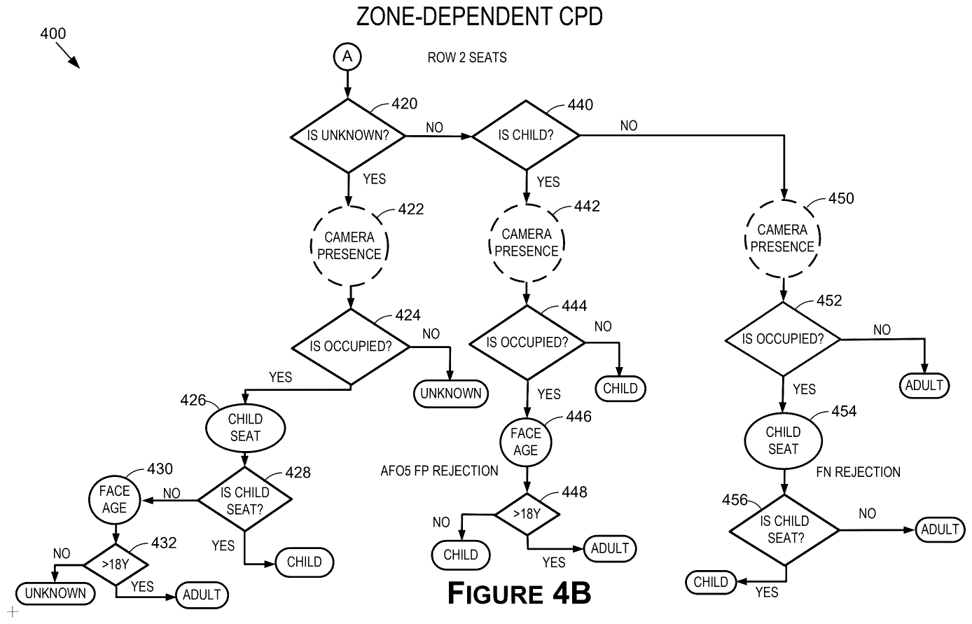

FIGS. 4A and 4B illustrate an example state machine 400 which may be used to fuse one or more child presence detection results for one or more occupant slots such as those in a second row of seating. At step 402, the state machine 400 may look up (or trigger generation of) a RADAR blockage detection result (e.g., a detection confidence, a binary result), and at step 404, the state machine 400 may evaluate whether the RADAR blockage detection result indicates that the RADAR sensor is not blocked (e.g., classified as not blocked with more than a threshold confidence). If the state machine 400 determines the RADAR sensor is not blocked, and taking an occupant slot such as one of the seats in the second row of seating as an example, at step 406, the state machine 400 may look up (or trigger generation of) a RADAR liveness detection result for that slot (e.g., a detection confidence, a binary result). At step 408, the state machine 400 may evaluate whether the RADAR liveness detection indicates the occupant slot is occupied by a live occupant (e.g., a positive detection with more than a threshold confidence). If not, the state machine 400 may classify the slot as empty. If so, at step 410, the state machine 400 may look up (or trigger generation of) a RADAR pet detection result for that slot (e.g., a detection confidence, a binary result), and at step 412, the state machine 400 may evaluate whether the RADAR pet detection result indicates the presence of a human-like signature in that slot (e.g., classified more than a threshold confidence). If not, the state machine 400 may classify the slot as occupied by a live occupant with a non-human-like signature (e.g., a pet). If so, at step 414, the state machine 400 may look up (or trigger generation of) a RADAR child presence classification result for that slot (e.g., a detection confidence, a binary result).

At step 420, the state machine 400 may evaluate whether the RADAR child presence classification was inconclusive or unknown (e.g., no classification was made with at least a threshold confidence). If so, at step 422, the state machine 400 may look up (or trigger generation of) a camera presence or occupancy detection result for that slot (e.g., a detection confidence, a binary result), and at step 424, the state machine 400 may evaluate whether the camera presence or occupancy detection indicates the occupant slot is occupied (e.g., a positive detection with more than a threshold confidence). If so, at step 426, the state machine 400 may look up (or trigger generation of) a child seat detection result for that slot (e.g., a detection confidence, a binary result), and at step 428, the state machine 400 may evaluate whether the child seat detection indicates the occupant slot is occupied by a child seat (e.g., a positive detection with more than a threshold confidence). If the state machine 400 determines the slot is occupied by a child seat, the state machine 400 may classify the slot as occupied by a child (e.g., and generate a corresponding representation). Otherwise, at step 430, the state machine 400 may look up (or trigger generation of) a camera face-based age estimation result for that slot (e.g., a regressed age or classified age range), and at step 432, the state machine 400 may evaluate whether the camera face-based age estimation result indicates the occupant slot is occupied by an adult (e.g., a regressed age or classified age range above 18 years old). If so, the state machine 400 may classify the slot as occupied by an adult (e.g., and generate a corresponding representation). Otherwise, the state machine 400 classify the slot as empty (e.g., and generate a corresponding representation).

In some embodiments (e.g., if the state machine 400 determined that the RADAR child presence classification was not inconclusive or unknown at step 420), at step 440, the state machine 400 may evaluate whether the RADAR child presence classification indicates the occupant slot is occupied by a child (e.g., a positive detection with more than a threshold confidence). If so, at step 442, the state machine 400 may look up (or trigger generation of) a camera presence or occupancy detection result for the occupant slot (e.g., a detection confidence, a binary result), and at step 444, the state machine 400 may evaluate whether the camera presence or occupancy detection indicates the occupant slot is occupied (e.g., a positive detection with more than a threshold confidence). If not, the state machine 400 may classify the slot as occupied by a child (e.g., and generate a corresponding representation). If so, at step 446, the state machine 400 may look up (or trigger generation of) a camera face-based age estimation result for that slot (e.g., a regressed age or classified age range), and at step 448, the state machine 400 may evaluate whether the camera face-based age estimation result indicates the occupant slot is occupied by an adult (e.g., a regressed age or classified age range above 18 years old). If so, the state machine 400 may classify the slot as occupied by an adult (e.g., and generate a corresponding representation). Otherwise, the state machine 400 classify the slot as empty (e.g., and generate a corresponding representation).

In some embodiments (e.g., if the state machine 400 determined that the RADAR child presence classification indicates the occupant slot is not unknown and not occupied by a child at step 440, or indicates the occupant slot is occupied by an adult), at step 450, the state machine 400 may look up (or trigger generation of) a camera presence or occupancy detection result for the occupant slot (e.g., a detection confidence, a binary result), and at step 452, the state machine 400 may evaluate whether the camera presence or occupancy detection indicates the occupant slot is occupied (e.g., a positive detection with more than a threshold confidence). If not, the state machine 400 may classify the slot as occupied by an adult (e.g., and generate a corresponding representation). If so, at step 454, the state machine 400 may look up (or trigger generation of) a child seat detection result for that slot (e.g., a detection confidence, a binary result), and at step 456, the state machine 400 may evaluate whether the child seat detection indicates the occupant slot is occupied by a child seat (e.g., a positive detection with more than a threshold confidence). If so, the state machine 400 may classify the slot as occupied by a child (e.g., and generate a corresponding representation). If not, the state machine 400 may classify the slot as occupied by an adult (e.g., and generate a corresponding representation).

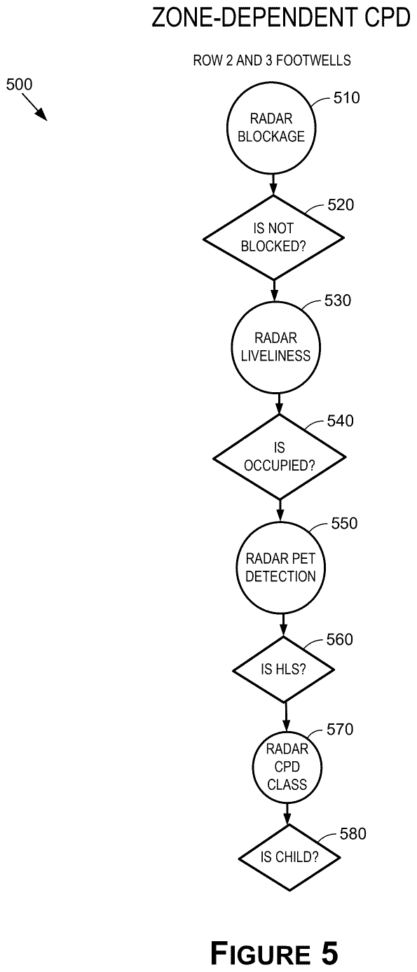

FIG. 5 illustrates an example state machine 500 which may be used to fuse one or more child presence detection results for one or more occupant slots such as those in a second and third rows of footwells. At step 510, the state machine 500 may look up (or trigger generation of) a RADAR blockage detection result (e.g., a detection confidence, a binary result), and at step 520, the state machine 500 may evaluate whether the RADAR blockage detection result indicates that the RADAR sensor is not blocked (e.g., classified as not blocked with more than a threshold confidence). If the state machine 500 determines the RADAR sensor is not blocked, and taking an occupant slot such as a footwell in some row of vehicle seating outside of the first row as an example, at step 530, the state machine 500 may look up (or trigger generation of) a RADAR liveness detection result for that slot (e.g., a detection confidence, a binary result), and at step 540, the state machine 500 may evaluate whether the RADAR liveness detection indicates the occupant slot is occupied by a live occupant (e.g., a positive detection with more than a threshold confidence). If so, at step 550, the state machine 500 may look up (or trigger generation of) a RADAR pet detection result for that slot (e.g., a detection confidence, a binary result), and at step 560, the state machine 500 may evaluate whether the RADAR pet detection result indicates the presence of a human-like signature in that slot (e.g., classified more than a threshold confidence). If so, at step 570, the state machine 500 may look up (or trigger generation of) a RADAR child presence classification for that slot (e.g., a detection confidence, a binary result). At step 580, the state machine 500 may evaluate whether the RADAR child presence classification indicates the occupant slot is occupied by a child (e.g., a positive detection with more than a threshold confidence). If so, the state machine 500 may classify the slot as occupied by a child.

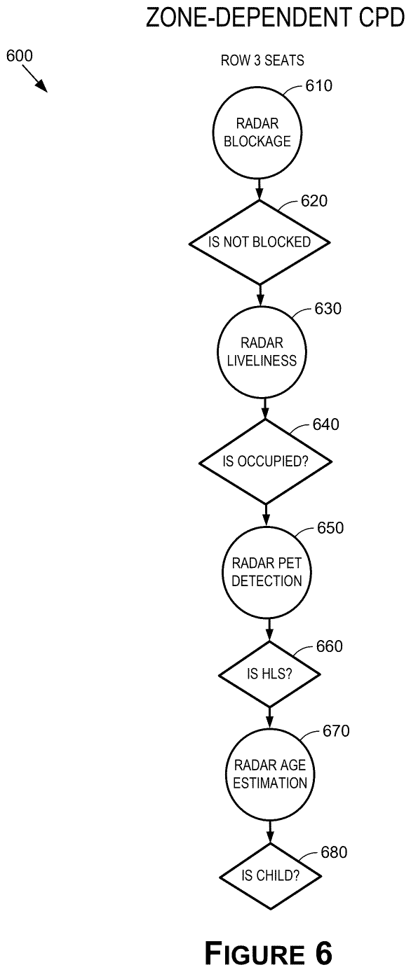

FIG. 6 illustrates an example state machine 600 which may be used to fuse one or more child presence detection results for one or more occupant slots such as those in a third row of seating. At step 610, the state machine 600 may look up (or trigger generation of) a RADAR blockage detection result (e.g., a detection confidence, a binary result), and at step 620, the state machine 600 may evaluate whether the RADAR blockage detection result indicates that the RADAR sensor is not blocked (e.g., classified as not blocked with more than a threshold confidence). If the state machine 600 determines the RADAR sensor is not blocked, and taking an occupant slot such as a seat in the third (or higher) row of vehicle seating as an example, at step 630, the state machine 600 may look up (or trigger generation of) a RADAR liveness detection result for that slot (e.g., a detection confidence, a binary result), and at step 640, the state machine 600 may evaluate whether the RADAR liveness detection indicates the occupant slot is occupied by a live occupant (e.g., a positive detection with more than a threshold confidence). If so, at step 650, the state machine 600 may look up (or trigger generation of) a RADAR pet detection result for that slot (e.g., a detection confidence, a binary result), and at step 660, the state machine 600 may evaluate whether the RADAR pet detection result indicates the presence of a human-like signature in that slot (e.g., classified more than a threshold confidence). If so, at step 670, the state machine 600 may look up (or trigger generation of) a RADAR age estimation result for that slot (e.g., a regressed age or classified age range), and at step 680, the state machine 600 may evaluate whether the RADAR age estimation result indicates the occupant slot is occupied by a child (e.g., a regressed age or classified age range below 18 years old). If so, the state machine 600 may classify the slot as occupied by a child.

In some embodiments, the sensor fusion logic 140 in the base control stack 110 may select, combine, weight, and/or apply threshold(s) to signals corresponding to the sensor data 120a-n, one or more outputs of the detection pipeline(s) 130a-n (e.g., derived values, detection confidence(s) indicating a likelihood that one or more conditions were detected, etc.) using (e.g., slot-dependent) weights, thresholds, flags, values, and/or other parameters (e.g., stored in memory accessible to the base control stack 110, or in code of the base control stack 110). In some embodiments, the applicable detection pipeline(s) 130a-n and/or the corresponding weights, thresholds, flags, values, and/or other parameters may be tailored or specific to one or more occupant slots (e.g., vehicle seats, rows of seating, footwells, rows of footwells, etc.). For example, corresponding signals (e.g., measured sensor values, binary detection values, detection confidence values, etc.) may be generated for each supported occupant slot, and the sensor fusion logic 140 may look up the corresponding weights, thresholds, flags, values, and/or other parameters for a particular slot(s) (e.g., from one or more look up tables or other data structure(s)) and may select, combine, weight, and/or apply threshold(s) to the corresponding signals using the weights, thresholds, flags, values, and/or other parameters for that slot(s). The weights, thresholds, flags, values, and/or other parameters may be configurable (e.g., per slot), and may be uploaded to and/or stored in any suitable location (e.g., in memory, code, etc.) on the ego-machine compute platform 100 (e.g., provided in advance by an OEM via the application layer API(s) 160, middleware API(s), developer API(s), diagnostic API(s), etc. for real-time use by the base control stack 110).

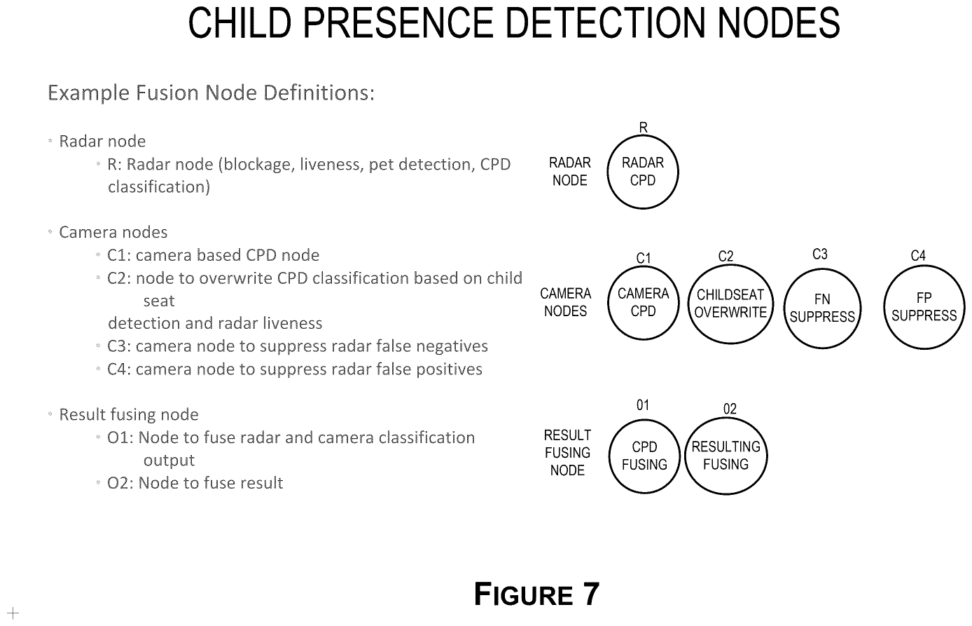

Taking child presence detection as an example, FIGS. 7-14 illustrate some examples of configurable sensor fusion for child presence detection which may be implemented in the detection pipeline(s) 130a-n and/or the sensor fusion logic 140 for one or more occupant slots, in accordance with some embodiments of the present disclosure. The following examples use nodes to represent possible (e.g., slot-dependent) logic of the detection pipeline(s) 130a-n (or lookups of corresponding detection results) and/or of state machines that fuse corresponding detection results to generate a unified representation of—or otherwise determine—whether the applicable condition (e.g., child presence) was detected. More specifically, FIG. 7 defines some example child presence detection nodes which may be used for configurable sensor fusion, including RADAR node(s) (e.g., representing generation or look up of results of detection(s) performed by corresponding detection pipeline(s) 130a-n based on RADAR data), and camera node(s) (e.g., representing generation or look up of results of detection(s) performed by corresponding detection pipeline(s) 130a-n based on image data, and/or one or more state machines implementing fusion logic), and result fusing nodes (e.g., representing fusion of node inputs, as illustrated in subsequent figures).

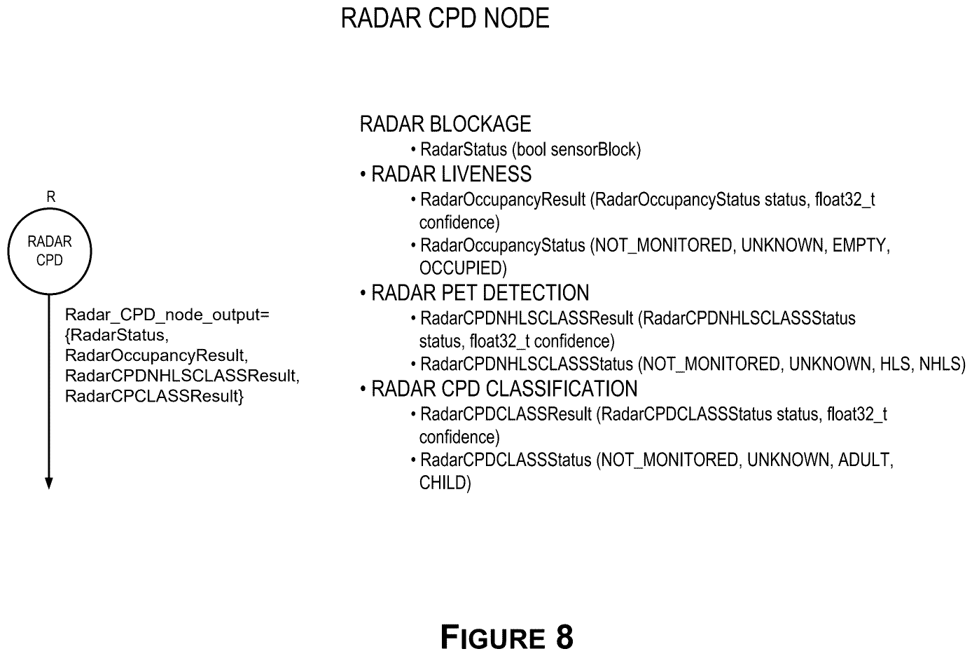

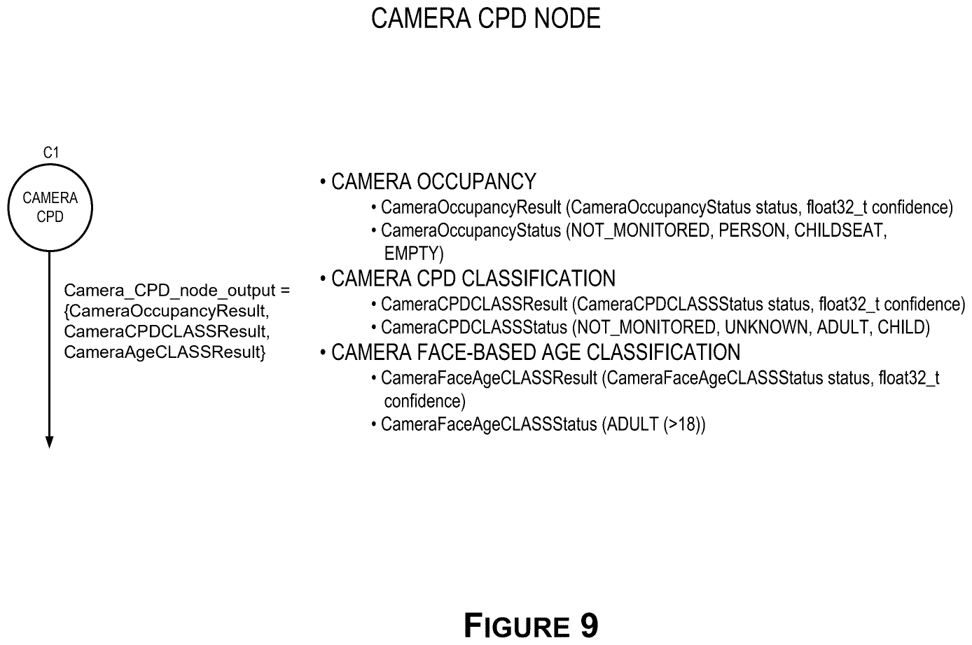

More specifically, FIG. 8 illustrates some possible outputs from an example RADAR CPD node, in accordance with some embodiments of the present disclosure. For example, the RADAR CPD node may represent generation or look up one or more types of RADAR detection results (e.g., detected class(es), detection confidence(s), etc. detected based on RADAR data), such as results of RADAR blockage detection, RADAR liveness detection, RADAR pet detection, and/or RADAR child presence classification. FIG. 9 illustrates some possible outputs from an example camera CPD node, in accordance with some embodiments of the present disclosure. For example, the camera CPD node may represent generation or look up camera detection results (e.g., detected class(es), detection confidence(s), etc. detected based on image data), such as results of camera presence or occupancy detection, camera child presence classification, and/or camera face-based age classification.

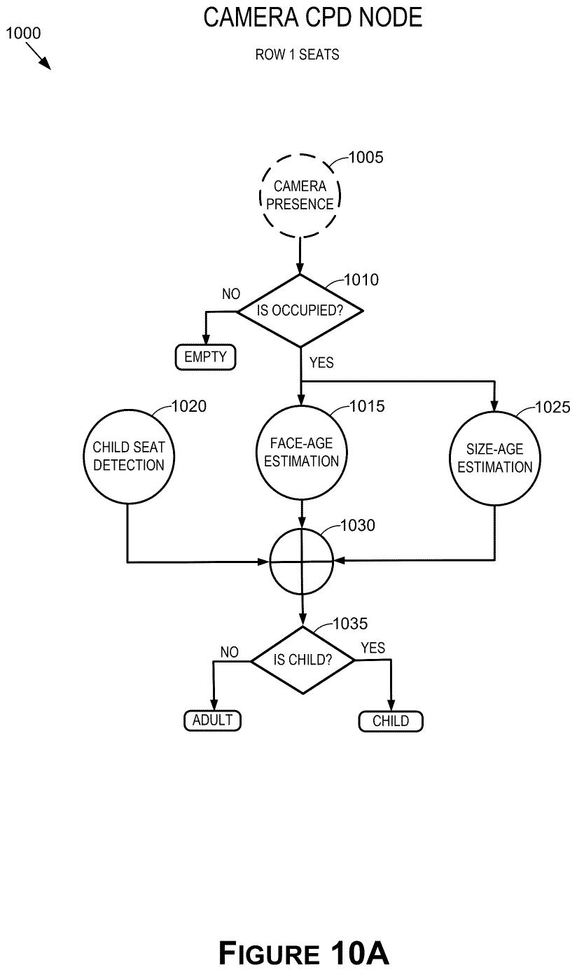

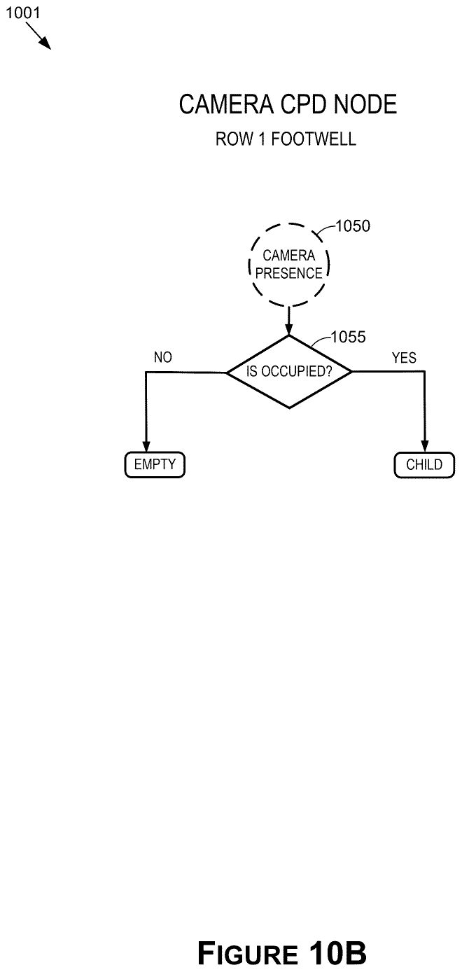

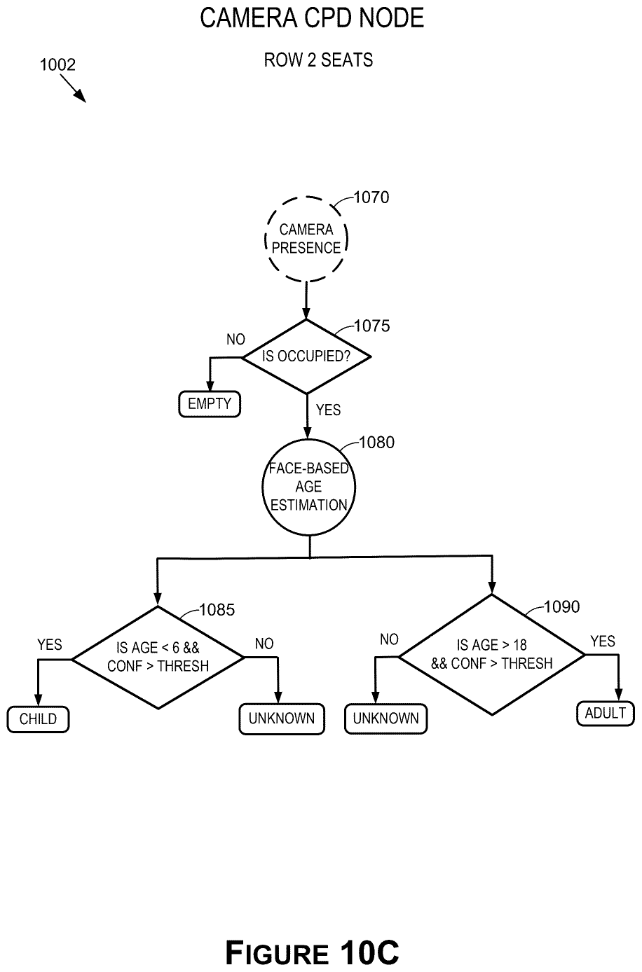

In some embodiments, the camera CPD node may represent generation or look up of results of a (e.g., slot-dependent) state machine (e.g., which may be implemented by a corresponding detection pipeline 130a-n and/or sensor fusion logic 140 of FIG. 1 and) that combines different types of camera detection results (detected based on image data). For example, FIG. 10A illustrates an example state machine 1000 which may be implemented by a camera CPD node and which may be used to fuse one or more camera detection results for one or more occupant slots such as those in a first row of seating. Taking an occupant slot such as the passenger's seat as an example, at step 1005, the state machine 1000 may look up (or trigger generation of) a camera presence or occupancy detection result for that slot (e.g., a detection confidence, a binary result), and at step 1010, the state machine 1000 may evaluate whether the camera presence or occupancy detection indicates the occupant slot is occupied (e.g., a positive detection with more than a threshold confidence). If not, the state machine 1000 may classify the slot as empty (a camera detection result). Otherwise, at step 1015, the state machine 1000 may look up (or trigger generation of) a camera face-based age estimation result for that slot (e.g., a regressed age or classified age range), and at step 1025, the state machine 1000 may look up (or trigger generation of) a camera size-based age estimation result for that slot (e.g., a regressed age or classified age range). At step 1020, the state machine 1000 may look up (or trigger generation of) a child seat detection result for that slot (e.g., a detection confidence, a binary result), and at step 1030, the state machine 1000 may combine the results of steps 1015, 1020, and 1025 in any suitable manner to generate a unified representation of - or otherwise determine - whether the occupant slot is occupied by a child. For example, the state machine 300 may generate a unified detection confidence as a weighted combination of corresponding detection confidences. As such, at step 1035, the state machine 1000 may determine whether the slot is occupied by a child (e.g., whether the unified detection confidence is at least a threshold confidence). If so, the state machine 1000 may classify the slot as occupied by a child (e.g., and generate a corresponding representation). Otherwise, the state machine 1000 may classify the slot as occupied by an adult (e.g., and generate a corresponding representation).

FIG. 10B illustrates an example state machine 1001 which may be implemented by a camera CPD node and which may be used to fuse one or more camera detection results for one or more occupant slots such a first row footwell. At step 1050, the state machine 1001 may look up (or trigger generation of) a camera presence or occupancy detection result for an applicable occupant slot (e.g., a detection confidence, a binary result), and at step 1055, the state machine 1001 may evaluate whether the camera presence or occupancy detection indicates the occupant slot is occupied (e.g., a positive detection with more than a threshold confidence). If not, the state machine 1001 may classify the slot as empty (e.g., and generate a corresponding representation). Otherwise, the state machine 1001 may classify the slot as occupied by a child (e.g., and generate a corresponding representation).

FIG. 10C illustrates an example state machine 1002 which may be implemented by a camera CPD node and which may be used to fuse one or more camera detection results for one or more occupant slots such as those in a second row of seating. Taking an occupant slot such as a seat in the second row as an example, at step 1070, the state machine 1002 may look up (or trigger generation of) a camera presence or occupancy detection result for that slot (e.g., a detection confidence, a binary result), and at step 1075, the state machine 1002 may evaluate whether the camera presence or occupancy detection indicates the occupant slot is occupied (e.g., a positive detection with more than a threshold confidence). If not, the state machine 1002 may classify the slot as empty (e.g., and generate a corresponding representation). Otherwise, at step 1080, the state machine 1002 may look up (or trigger generation of) a camera face-based age estimation result for that slot (e.g., a regressed age or classified age range). At step 1085, the state machine 1002 may evaluate whether the camera face-based age estimation result indicates the age of the occupant in the slot is less than some age (e.g., 6 years old) (e.g., with at least a threshold confidence), and if so, the state machine 1002 may classify the slot as occupied by a child (e.g., and generate a corresponding representation). Otherwise, the state machine 1002 may classify the slot as unknown. At step 1090, the state machine 1002 may evaluate whether the camera face-based age estimation result indicates the age of the occupant in the slot is greater than some age (e.g., 18 years old) (e.g., with at least a threshold confidence), and if so, the state machine 1002 may classify the slot as occupied by an adult. Otherwise, the state machine 1002 may classify the slot as unknown.

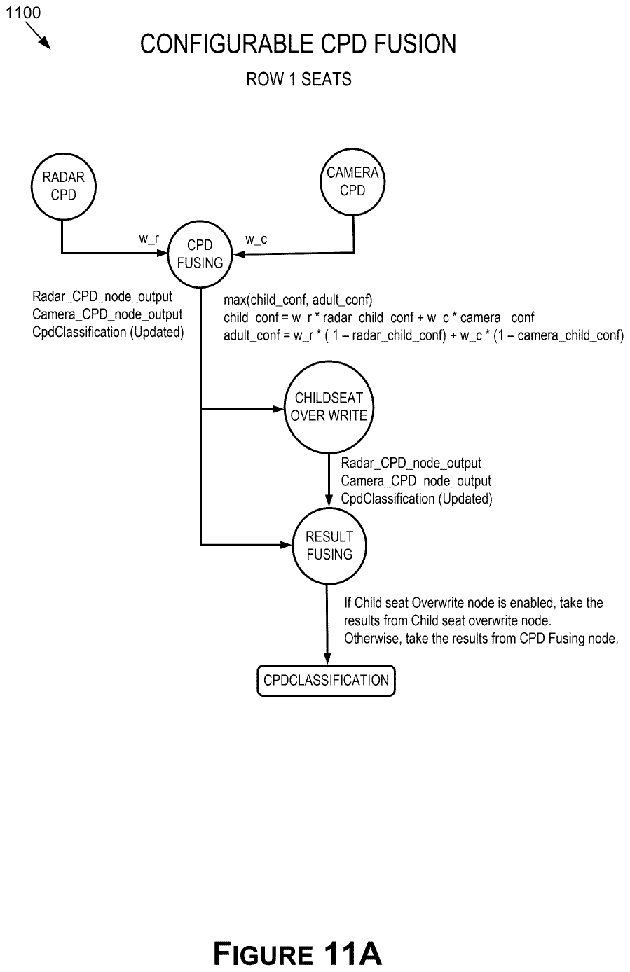

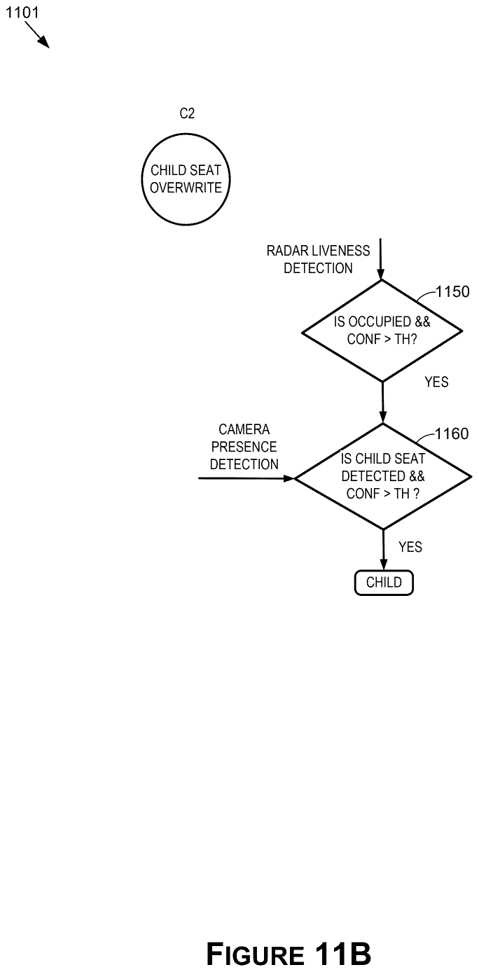

The example classifications of an occupant slot (e.g., occupied by a child, occupied by an adult, empty, unknown) described with respect to FIGS. 10A-10C represent possible camera detection results (detected based on image data). FIG. 11A illustrates an example state machine 1100 which may be used to fuse RADAR and camera detection results for one or more occupant slots such as those in a first row of seating. For example, FIG. 11A illustrates a CPD fusing node (e.g., which may be implemented by the sensor fusion logic 140 of FIG. 1 and) which may fuse RADAR detection results output by the RADAR CPD node with camera detection results output by the camera CPD node. For example, at the CPD fusing node, the state machine 1100 may look up relative (e.g., configurable) weights wᵣ and w_{c} for combining RADAR and camera detection confidences and/or (e.g., configurable) temporal offset value(s) for aligning the timestamps of corresponding RADAR and camera detection results (e.g., any of which may be tailored or specific to the applicable occupant slot such as first a seat in the first row of seating) and may use them generate a unified detection confidence as a weighted combination of corresponding RADAR and camera detection confidences. By way of nonlimiting example, the state machine 1100 may look up the confidence (output by the RADAR CPD node) that a child was detected in the occupant slot from RADAR data (radar_child_conf) (e.g., corresponding to a timestamp computed using a configurable temporal offset), look up the confidence (output by the camera CPD node) that a child was detected in the occupant slot from image data (camera_child_conf) (e.g., corresponding to a timestamp computed using a configurable temporal offset), and compute a unified detection confidence as:

$$child\_conf = {w}_{r} * radar\_child \_conf + {w}_{c} * camera\_conf$$

$$adult\_conf = {w}_{r} * ( 1 - radar\_child \_conf ) + {w}_{c} * ( 1 - camera\_child \_conf )$$

$$CpdClassification ( Updated ) = max ( child\_conf , adult\_conf )$$

Additionally or alternatively, the state machine 1100 may look up a (e.g., configurable) flag, property, value, or other parameter designing whether or not to execute lotic represented by the child seat overwrite node in FIG. 11A. If the child seat overwrite node (or corresponding functionality) is enabled, the state machine 1100 execute logic represented by the child seat overwrite node and update the unified detection confidence.

For example, FIG. 11B illustrates an example state machine 1101 which may be implemented by a child seat overwrite node and may be used to overwrite a missed child presence detection. For example, the state machine 1101 may look up (or trigger generation of) a RADAR liveness detection result for the applicable slot (e.g., output by the RADAR CPD node illustrated in FIG. 11A), and at step 1150, the state machine 1101 may evaluate whether the RADAR liveness detection indicates the occupant slot is occupied by a live occupant (e.g., a positive detection with more than a threshold confidence). In some embodiments, the state machine 1101 may look up a configurable threshold confidence for RADAR liveness detection and apply the configured threshold confidence to evaluate whether the slot is occupied by a live occupant. If so, at step 1160, the state machine 1101 may look up (or trigger generation of) a child seat detection result for that slot (e.g., a detection confidence, a binary result) and evaluate whether the child seat detection result indicates the occupant slot is occupied by a child seat (e.g., a positive detection with more than a threshold confidence). In some embodiments, the state machine 1101 may look up a configurable threshold confidence for child seat detection and apply the configured threshold confidence to evaluate whether the slot is occupied by a child seat. If so, the state machine 1102 may classify the slot as occupied by a child (e.g., updating the CpdClassification to represent a representative positive classification confidence, the camera presence or occupancy detection confidence, the RADAR liveness detection confidence, some combination thereof, etc.).

As such, and returning to FIG. 11A, at the result fusing node, the state machine 1100 may determine whether the child seat overwrite node is enabled, and if so, use the output of the child seat overwrite node (e.g., the updated detection confidence) as the unified representation of whether a child was detected in the occupant slot. Otherwise, the state machine 1100 may use the output of the CPD fusing node as the unified representation of whether a child was detected in the occupant slot.

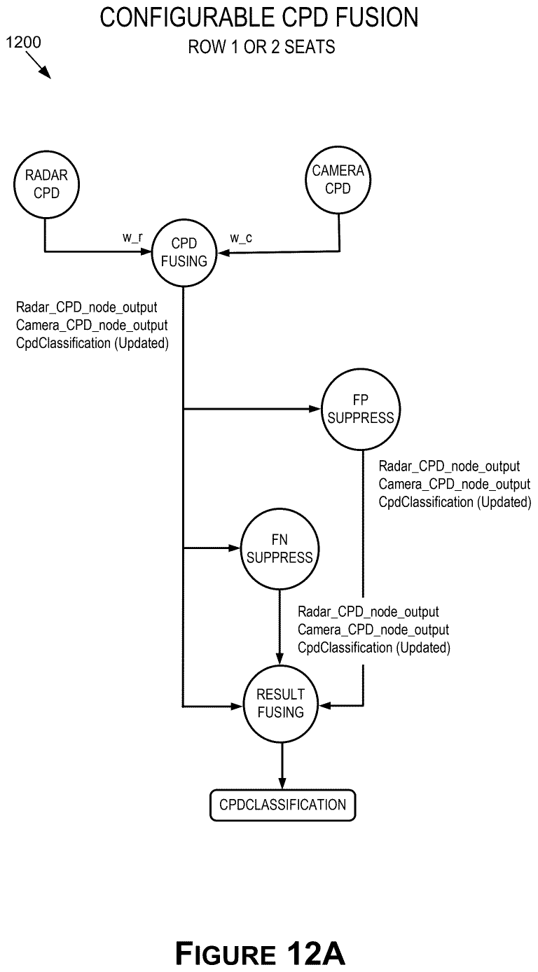

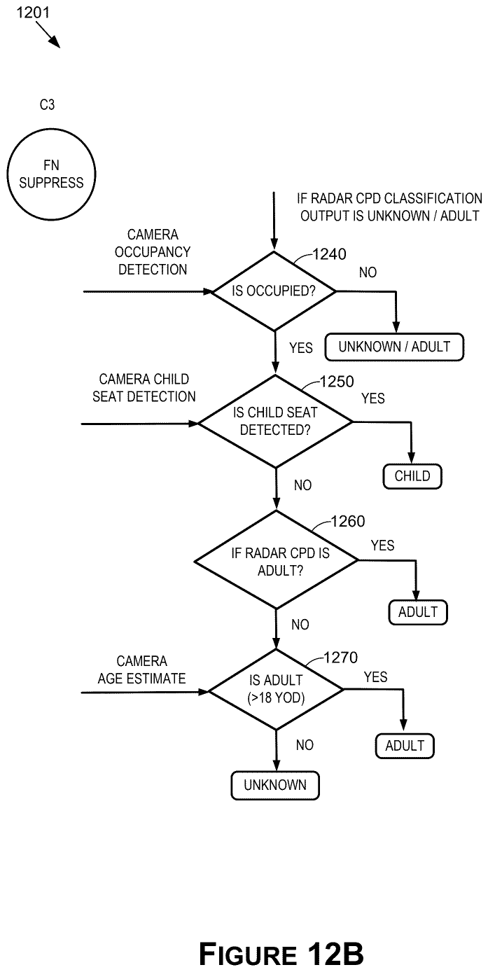

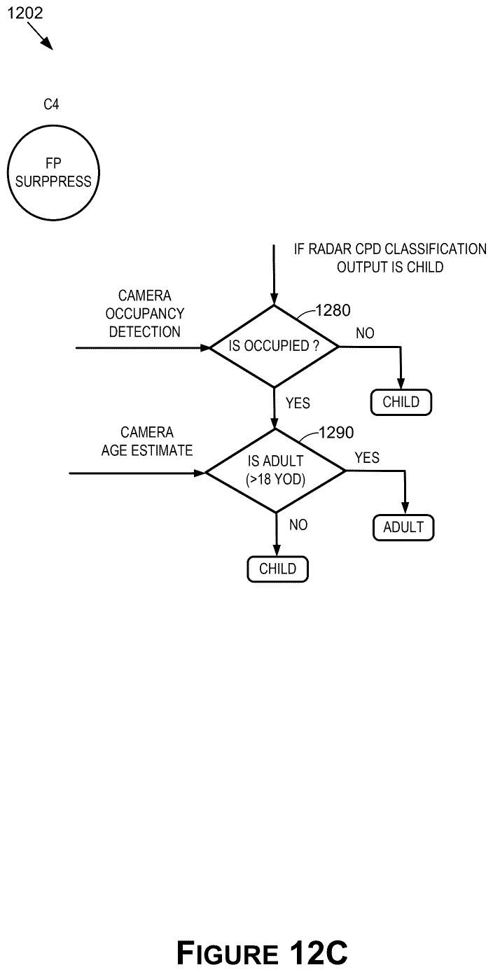

FIG. 12A illustrates an example state machine 1200 which may be used to fuse RADAR and camera detection results for one or more occupant slots such as those in a first or second row of seating. In some embodiments, the state machine 1200 executes the RADAR CPD node, the camera CPD node, and the CPD fusing node illustrated in FIG. 12A similarly as the state machine 1100 of FIG. 11. In this example, false positive and/or false negative suppression logic (e.g., represented by corresponding false positive and false negative suppression nodes in FIG. 12A) may be toggled by setting a corresponding (e.g., slot-dependent) flag, property, value, or other parameter. In an example implementation, if the child presence classification output by the CPD fusing node classifies an occupant slot as occupied by an adult (e.g., with at least a threshold confidence) or unknown, the state machine 1200 may look up a (e.g., configurable) flag, property, value, or other parameter designating whether or not to execute logic represented by the false negative suppression node, and if the false negative suppression node (or corresponding functionality) is enabled, the state machine 1200 may execute corresponding functionality (e.g., the state machine 1201 of FIG. 12B), and the state machine 1200 may use the output from the false negative suppression node (e.g., an updated binary classification, an updated detection confidence, etc.) as the output of the result fusing node. If the child presence classification output by the CPD fusing node classifies the occupant slot as occupied by an adult or unknown and the false negative suppression node (or corresponding functionality) is disabled, the state machine 1200 may use the output from the CPD fusing node as the output of the result fusing node.

If the child presence classification output by the CPD fusing node classifies the occupant slot as occupied by a child (e.g., with at least a threshold confidence), the state machine 1200 may look up a (e.g., configurable) flag, property, value, or other parameter designating whether or not to execute logic represented by the false positive suppression node, and if the false positive suppression node (or corresponding functionality) is enabled, the state machine 1200 may execute the corresponding functionality (e.g., the state machine 1202 of FIG. 12C), and the state machine 1200 may use the output from the false positive suppression node (e.g., an updated binary classification, an updated detection confidence, etc.) as the output of the result fusing node. If the child presence classification output by the CPD fusing node classifies the occupant slot as occupied by a child and the false positive suppression node (or corresponding functionality) is disabled, the state machine 1200 may use the output from the CPD fusing node as the output of the result fusing node.

FIG. 12B illustrates an example state machine 1201 which may be implemented by a false negative suppression node and may be used to overwrite a missed child presence detection. For example, the state machine 1201 may look up a RADAR child presence classification for the applicable occupant slot (e.g., a detection confidence, a binary result) and evaluate whether the RADAR child presence classification indicates the occupant slot is occupied by an adult (e.g., a positive detection with more than a threshold confidence) or is unknown. If not, the state machine 1201 may terminate (e.g., returning to the state machine 1200) without updating the child presence classification. If so, the state machine 1201 may look up a camera presence or occupancy detection result for that slot (e.g., a detection confidence, a binary result), and at step 1240, the state machine 1201 may evaluate whether the camera presence or occupancy detection result indicates the occupant slot is occupied (e.g., a positive detection with more than a threshold confidence). If not, the state machine 1201 may terminate (e.g., returning to the state machine 1200) without updating the child presence classification. Otherwise, the state machine 1201 may lookup a child seat detection result for that slot (e.g., a detection confidence, a binary result), and at step 1250, the state machine 1201 may evaluate whether the child seat detection result indicates the occupant slot is occupied by a child seat (e.g., a positive detection with more than a threshold confidence). If so, the state machine 1201 may classify the slot as occupied by a child (e.g., updating the CpdClassification to represent a representative positive classification confidence, the camera presence or occupancy detection confidence, the child seat detection confidence, some combination thereof, etc.).

Otherwise (e.g., a child seat was not detected), the state machine 1201 may evaluate whether the RADAR child presence classification indicates the occupant slot is occupied by an adult (e.g., a positive detection with more than a threshold confidence). If so, the state machine 1201 may classify the slot as occupied by an adult (e.g., if necessary, updating the CpdClassification to represent a positive classification confidence, the camera presence or occupancy detection confidence, the child seat detection confidence, some combination thereof, etc.).

If the RADAR child presence classification does not indicate the occupant slot is occupied by an adult, the state machine 1201 may look up camera age estimation result (e.g., camera face-based age estimation result, camera size-based age estimation result) for that slot (e.g., a regressed age or classified age range), and at step 1270, the state machine 1201 may evaluate whether the camera age estimation result indicates the age of the occupant in the slot is greater than some age (e.g., 18 years old) (e.g., with at least a threshold confidence). If so, the state machine 1201 may classify the slot as occupied by an adult. Otherwise, the state machine 1002 may classify the slot as unknown.

In some embodiments, the state machine 1201 may look up and apply one or more configurable (e.g., slot-dependent) threshold confidences for camera presence or occupancy detection (e.g., at step 1240), child seat detection (e.g., at step 1250), RADAR child presence classification (e.g., at step 1260), and/or camera age estimation (e.g., at step 1270). As such, one or more of the threshold confidences applied by the state machine 1201 may be configurable, and may be configured differently for different occupant slots.

FIG. 12C illustrates an example state machine 1202 which may be implemented by a false positive suppression node and may be used to overwrite an incorrect child presence detection. For example, the state machine 1202 may look up a RADAR child presence classification for an applicable occupant slot (e.g., a detection confidence, a binary result) and evaluate whether the RADAR child presence classification indicates the occupant slot is occupied by a child (e.g., a positive detection with more than a threshold confidence). If not, the state machine 1202 may terminate (e.g., returning to the state machine 1200) without updating the child presence classification). If so, the state machine 1202 may look up a camera presence or occupancy detection result for that slot (e.g., a detection confidence, a binary result), and at step 1280, the state machine 1202 may evaluate whether the camera presence or occupancy detection result indicates the occupant slot is occupied (e.g., a positive detection with more than a threshold confidence). If not, the state machine 1202 may terminate (e.g., returning to the state machine 1200) without updating the child presence classification. Otherwise, the state machine 1202 may look up camera age estimation result (e.g., camera face-based age estimation result, camera size-based age estimation result) for that slot (e.g., a regressed age or classified age range), and at step 1290, the state machine 1202 may evaluate whether the camera age estimation result indicates the age of the occupant in the slot is greater than some age (e.g., 18 years old) (e.g., with at least a threshold confidence). If so, the state machine 1202 may classify the slot as occupied by an adult. Otherwise, the state machine 1002 may classify the slot as unknown.

In some embodiments, the state machine 1202 may look up and apply one or more configurable (e.g., slot-dependent) threshold confidences for camera presence or occupancy detection (e.g., at step 1280) and/or camera age estimation (e.g., at step 1290). As such, one or more of the threshold confidences applied by the state machine 1202 may be configurable, and may be configured differently for different occupant slots.

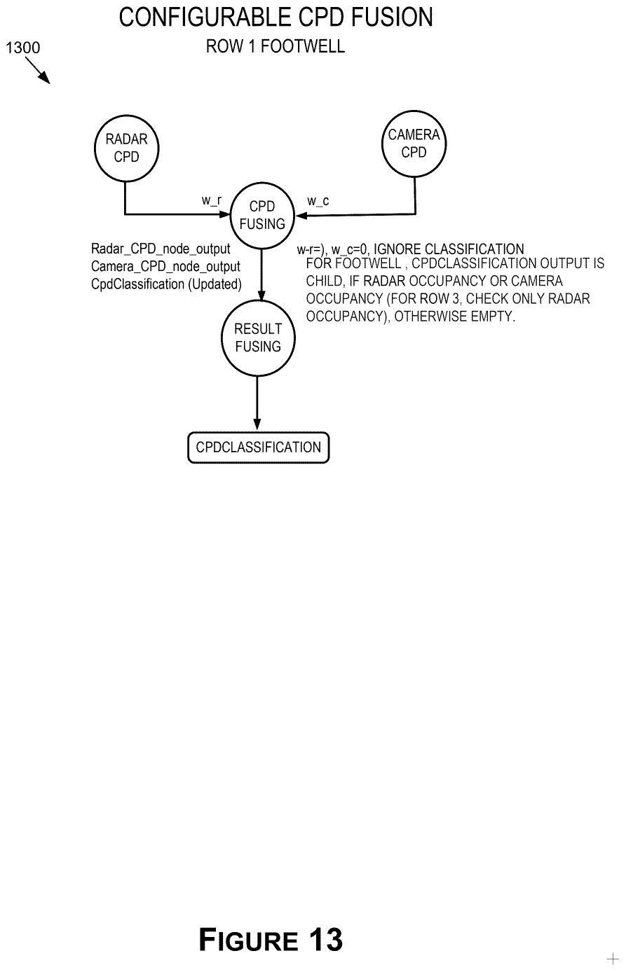

FIG. 13 illustrates an example state machine 1300 which may be used to fuse RADAR and camera detection results for one or more occupant slots such as a first row footwell. In some embodiments, the state machine 1300 executes the RADAR CPD node, the camera CPD node, and the CPD fusing node illustrated in FIG. 13 similarly as the state machine 1100 of FIG. 11 (e.g., using a configurable weight such as zero that ignores or cancels out the contribution from the camera CPD node for a first row footwell). In this example, the state machine 1300 may use the output from the CPD fusing node as the output of the state machine 1100 (e.g., as the output of the result fusing node). As such, the state machine 1300 illustrated in FIG. 13 represents a possible implementation in which the sensor fusion logic (e.g., the sensor fusion logic 140 of FIG. 1) may directly combine, weight, and/or apply threshold(s) to results from corresponding detection pipelines. Although FIG. 13 illustrates an example implementation in which the fusion logic combines different types of child presence detection results (e.g., RADAR and camera child presence classifications), similar (e.g., configurable) logic may additionally or alternatively be used to combine results from detection pipelines to generate corresponding unified detection results for other types of detection tasks, such as hands-on-wheel detection, body size detection, abnormal pose detection, heart rate monitoring, and/or liveness detection, to name a few examples. Generally, fusion logic may be implemented for any number and/or type of unified detection results (e.g., any combination of different types of unified detection results).

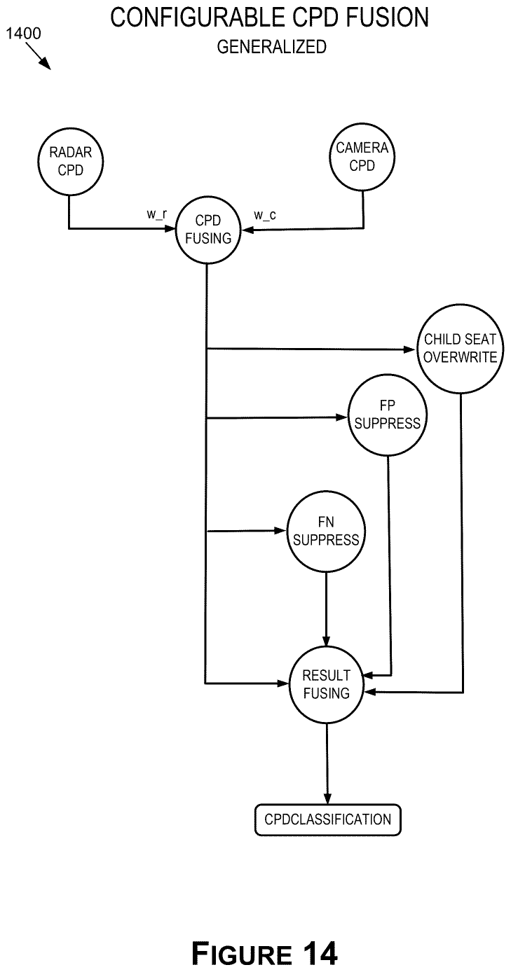

In some embodiments, sensor fusion logic (e.g., the sensor fusion logic 140 of FIG. 1) may use different state machines for different occupant slots. In contrast, some embodiments use the same logic (e.g., state machine) for each of multiple occupant slots, and may implement slot-dependent configurability using slot-dependent weights, thresholds, flags, values, and/or other parameters (e.g., represented in one or more look up tables). Continuing with child presence detection as an example, FIG. 14 illustrates an example generalized state machine 1400 which may be used to configure fuse RADAR and camera detection results for one or more occupant slots (e.g., all supported occupant slots). For example, the state machine 1400 may execute the RADAR CPD node, the camera CPD node, and the CPD fusing node illustrated in FIG. 14 similarly as the state machine 1100 of FIG. 11. The state machine 1400 may execute the child seat overwrite node similarly as the state machine 1100 of FIG. 11A and/or the state machine 1101 of FIG. 11B, the state machine 1400 may execute the negative positive suppression node similarly as the state machine 1200 of FIG. 12A and/or the state machine 1201 of FIG. 12B, and the state machine 1400 may execute the false positive suppression node similarly as the state machine 1200 of FIG. 12A and/or the state machine 1202 of FIG. 12C. As such, the state machine 1400 may use any suitable technique to combine the results of the enabled nodes. For example, if enabled, the child seat overwrite node and the false negative suppression node may both be considered to perform false negative suppression, so if both are enabled, the state machine 1400 may operate as an OR gate, using a positive classification identified by either the child seat overwrite node or the false negative suppression node and/or the higher resulting detection confidence as the output of the result fusing node. In some embodiments, the state machine 1400 may operate as an AND gate and only update the child presence classification if both the child seat overwrite node or the false negative suppression node identify a positive child presence classification. In some embodiments, the state machine 1400 may execute the result fusing node similarly as the state machine 1200 of FIG. 12A with respect to the false negative suppression node. As such, the state machine 1400 illustrated in FIG. 14 represents a possible implementation in which the same sensor fusion logic (e.g., the sensor fusion logic 140 of FIG. 1) may be used for different occupant slots (e.g., each supported occupant slot), and configurability may be achieved by the same sensor fusion logic looking up and applying configurable parameters which may be set to different values for different occupant slots.

In some embodiments, sensor fusion logic (e.g., the sensor fusion logic 140 of FIG. 1) may apply instance filtering for a given task, and the instance filtering may use one or more configurable parameters. For example, returning to FIG. 1, the detection pipeline(s) 130a-n and/or the sensor fusion logic 140 may operate at any suitable frame rate to generate a representation of a unified result of a corresponding detection task (e.g., a binary result, detection confidence). In some embodiments, the detection pipeline(s) 130a-n and/or the sensor fusion logic 140 may apply instance filtering to evaluate detections over multiple frames to determine which detections should be retained, merged, or discarded. In some embodiments, the sensor fusion logic 140 (and/or the detection pipeline(s) 130a-n) may look up and apply one or more configurable (e.g., slot-dependent) instance filtering parameters, such as window size (e.g., defining how many consecutive frames or time slices over which data is collected before confirming a detection) or one or more thresholds (e.g., a threshold confidence or number of consistent detections before confirming a detected object or condition, a threshold proximity for matching detections, a threshold velocity for filtering out unrealistic object movements, etc.). The instance filtering parameter(s) may be configurable (e.g., stored in a corresponding look up table, indexed, etc.) for each occupant slot and/or each supported task (e.g., object detection, presence or occupancy detection, child seat detection, age estimation, child presence classification, lane detection, etc.).

As such, the sensor fusion logic 140 may generate a representation of a unified result of a corresponding detection task (e.g., a binary result, detection confidence), and may provide a representation of the unified result to corresponding control component(s) 190 or otherwise instruct the corresponding control component(s) 190 to take a corresponding action. Generally, the applicable control component(s) 190 may depend on the detection task and/or the implementation. Taking automotive applications as an example, the control component(s) 190 may be part of an ADAS such as the ADAS system 1838 of FIG. 18C, and the control component(s) 190 may coordinate and/or manage one or more functions within the ADAS. Generally, the ADAS may use any known technique to assess the vehicle's surroundings, identify potential risks or hazards, and/or implement autonomous driving features such as adaptive cruise control, automatic emergency braking, lane-keeping assistance, and/or collision avoidance systems, to name a few examples.

Taking child presence detection as an example, the control component(s) 190 may determine that a unified result indicates a child is present in a particular occupant slot (e.g., a vehicle seat, a footwell). In some embodiments, based on detecting a child in a particular vehicle seat, the control component(s) 190 may trigger disabling of an airbag deployment for that seat and/or engage child lock settings. In some embodiments, based on determining that a child is present in any occupant slot, the control component(s) 190 may trigger a determination of whether the child is unattended using any known technique. The control component(s) 190 may determine whether at least one adult is present based on at least one occupant having a predicted age (e.g., estimated using any known technique) above a threshold, at least one occupant having a predicted height (e.g., a height derived from a size of one or more predicted limb lengths) above a threshold (e.g., greater than 50% the size of an average 10 year old), and/or otherwise. In some embodiments, based on determining that a detected child is unattended, the control component(s) 190 may trigger one or more actions (e.g., trigger an audible or visual alert, control temperature, unlock door(s), trigger or enable one or more camera feeds, etc.). These are just a few examples, and other actions may be implemented within the scope of the present disclosure.

Taking a DMS task such as driver drowsiness detection, distraction detection, or hands-on-wheel as an example, if the control component(s) 190 determine that a unified result indicates the driver is not attentive or in a drive-ready position, the control component(s) 190 may trigger one or more alerts (auditory, visual, or haptic) to regain the driver's attention, adjust one or more driver assistance features such as adaptive cruise control (e.g., increase the following distance or reduce the vehicle's speed) or lane keeping assistance (e.g., be more proactive in correcting lane deviations), trigger the ADAS to execute one or more safety interventions (e.g., automatic braking, emergency steering, transition to autonomous driving mode), adjust in-vehicle infotainment settings to minimize distractions, etc.

Taking some example OMS tasks, if the control component(s) 190 determine that a unified result indicates the presence of an occupant in a particular occupant slot, the control component(s) 190 may enable safety features for the slot such as airbags (for detected adults) or seatbelt reminders, or activate systems such as climate control and/or infotainment. If the control component(s) 190 determine that a unified result indicates detection of a known occupant, the control component(s) 190 may adjust settings such as seat position or temperature to saved preferences. If the control component(s) 190 determine that a unified result represents a detected body size of an occupant, the control component(s) 190 may tailor a corresponding seat position, seatbelt tension, and/or airbag deployment based on the detected body size. If the control component(s) 190 determine that a unified result indicates a detected abnormal pose (e.g., slumping forward, lying down unexpectedly, or showing signs of discomfort), an abnormal heart rate, and/or a change in detected liveness of a tracked occupant indicating potential distress, the control component(s) 190 may trigger some emergency response (e.g., contacting emergency services, displaying or announcing emergency instructions) and/or one or more safety interventions (e.g., automatic braking, emergency steering, transition to autonomous driving mode). These are just a few examples, and other types of detection tasks and responsive actions may be implemented within the scope of the present disclosure.

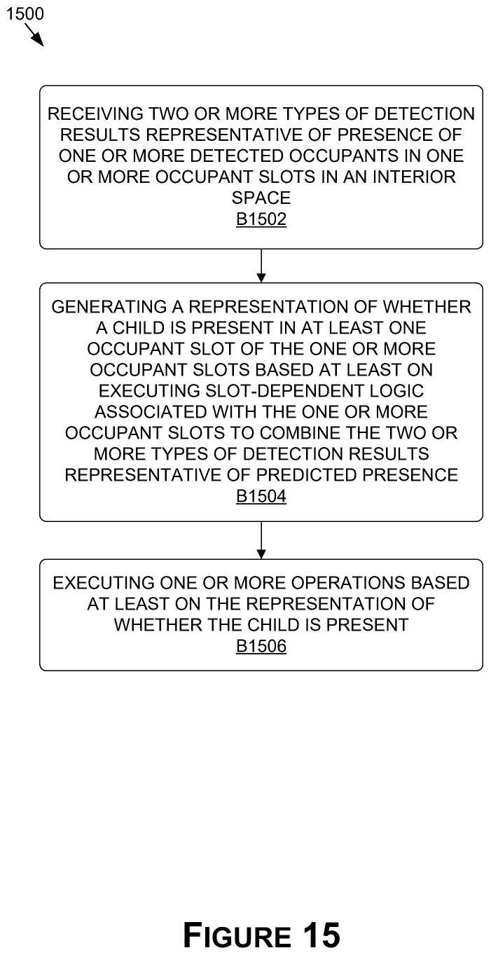

Now referring to FIGS. 15-17, each block of methods 1500-1700, described herein, comprises a computing process that may be performed using any combination of hardware, firmware, and/or software. For instance, various functions may be carried out by a processor executing instructions stored in memory. The methods may also be embodied as computer-usable instructions stored on computer storage media. The methods may be provided by a standalone application, a standalone service, a hosted service (standalone or in combination with another hosted service), or a plug-in to another product, to name a few. In addition, the methods 1500-1700 may be described, by way of example, with respect to the example ego-machine compute platform 100 of FIG. 1. However, these methods may additionally or alternatively be executed by any one system, or any combination of systems, including, but not limited to, those described herein.

FIG. 15 is a flow diagram showing a method 1500 for child presence detection with slot-dependent logic, in accordance with some embodiments of the present disclosure. The method 1500, at block B1502, includes receiving two or more types of detection results representative of presence of one or more detected occupants in one or more occupant slots in an interior space. For example, with respect FIG. 1, depending on the detection task and/or the implementation, different types of sensor(s), sensor data 120a-n, detection pipeline(s) 130a-n, and/or sensor fusion logic 140 may be involved. In some embodiments, the detection pipeline(s) 130a-n may use any known technique or other techniques described herein to perform RADAR blockage detection, RADAR liveness detection, RADAR pet detection, RADAR child presence or CPD classification, RADAR age estimation, camera presence or occupancy detection, camera child presence or CPD classification, child seat detection, camera face-based age estimation, and/or camera size-based age estimation. As such, the detection pipeline(s) 130a-n (or some other component of the base control stack 110) may provide the sensor fusion logic 140 with the corresponding detection results.

The method 1500, at block B1504, includes generating a representation of whether a child is present in at least one occupant slot of the one or more occupant slots based at least on executing slot-dependent logic associated with the one or more occupant slots to combine the two or more types of detection results representative of predicted presence. For example, with respect to FIG. 1, the sensor fusion logic 140 may use one or more (e.g., slot-dependent) state machines and/or (e.g., configurable, preset) parameters to select, combine, weight, and/or apply threshold(s) to (e.g., binary) signals or other representations of detection results (e.g., which may be representative of predicted presence) generated by a corresponding detection pipeline(s) 130a-n (e.g., detection confidence(s)) to generate a unified representation of whether a child (or adult) was detected in an occupant slot (e.g., a unified confidence, a binary result). FIGS. 2-6 illustrate some example (e.g., slot-dependent) state machines for child presence detection which may be implemented in the sensor fusion logic 140 (and/or the application layer 150) for one or more occupant slots, in accordance with some embodiments of the present disclosure.

The method 1500, at block B1506, includes executing one or more operations based at least on the representation of whether the child is present. For example, with respect to FIG. 1, the sensor fusion logic 140 may provide a representation of a unified result (e.g., a binary result, detection confidence) to corresponding control component(s) 190 or otherwise instruct the corresponding control component(s) 190 to take a corresponding action. Generally, the applicable control component(s) 190 may depend on the detection task and/or the implementation. Taking child presence detection as an example, the control component(s) 190 may determine that a unified result indicates a child is present in a particular occupant slot (e.g., a vehicle seat, a footwell). In some embodiments, based on detecting a child in a particular vehicle seat, the control component(s) 190 may trigger disabling of an airbag deployment for that seat and/or engage child lock settings. In some embodiments, based on determining that a child is present in any occupant slot, the control component(s) 190 may trigger a determination of whether the child is unattended using any known technique. The control component(s) 190 may determine whether at least one adult is present based on at least one occupant having a predicted age (e.g., estimated using any known technique) above a threshold, at least one occupant having a predicted height (e.g., a height derived from a size of one or more predicted limb lengths) above a threshold (e.g., greater than 50% the size of an average 10 year old), and/or otherwise. In some embodiments, based on determining that a detected child is unattended, the control component(s) 190 may trigger one or more actions (e.g., trigger an audible or visual alert, control temperature, unlock door(s), trigger or enable one or more camera feeds, etc.).

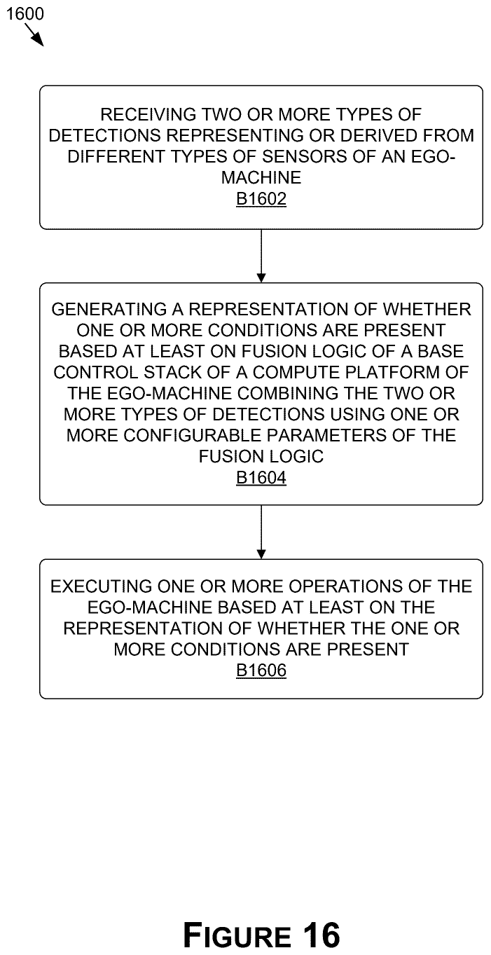

FIG. 16 is a flow diagram showing a method 1600 for detecting whether one or more conditions are present using fusion logic with one or more configurable parameters, in accordance with some embodiments of the present disclosure. The method 1600, at block B1602, includes receiving two or more types of detections representing or derived from different types of sensors of an ego-machine. For example, with respect to FIG. 1, depending on the detection task and/or the implementation, different types of sensor(s), sensor data 120a-n, and/or detection pipeline(s) 130a-n may be involved. In some embodiments, any known detection pipeline may be used to detect a condition from corresponding sensor data and generate a representation of the detected condition. Example detection tasks include object detection, driver drowsiness or distraction detection, hands-on-wheel detection, body size detection, abnormal pose detection, heart rate monitoring, liveness detection, and child presence detection, to name a few examples. As such, the detection pipeline(s) 130a-n (or some other component of the base control stack 110) may provide the sensor fusion logic 140 with the corresponding detection results.

The method 1600, at block B1604, includes generating a representation of whether one or more conditions are present based at least on fusion logic of a base control stack of a compute platform of the ego-machine combining the two or more types of detections using one or more configurable parameters of the fusion logic. For example, with respect to FIG. 1, the sensor fusion logic 140 may use one or more (e.g., slot-dependent) state machines and/or (e.g., configurable, preset) parameters to select, combine, weight, and/or apply threshold(s) to (e.g., binary) signals or other representations of detection results generated by a corresponding detection pipeline(s) 130a-n (e.g., detection confidence(s)) to generate a unified representation of whether the one or more conditions are presence (e.g., a unified confidence, a binary result). Depending on the detection task and/or the implementation, different types of sensor fusion logic 140 may be involved.

The method 1600, at block B1606, includes executing one or more operations of the ego-machine based at least on the representation of whether the one or more conditions are present. For example, with respect to FIG. 1, the sensor fusion logic 140 may provide a representation of a unified result (e.g., a binary result, detection confidence) to corresponding control component(s) 190 or otherwise instruct the corresponding control component(s) 190 to take a corresponding action. Generally, the applicable control component(s) 190 may depend on the detection task and/or the implementation.

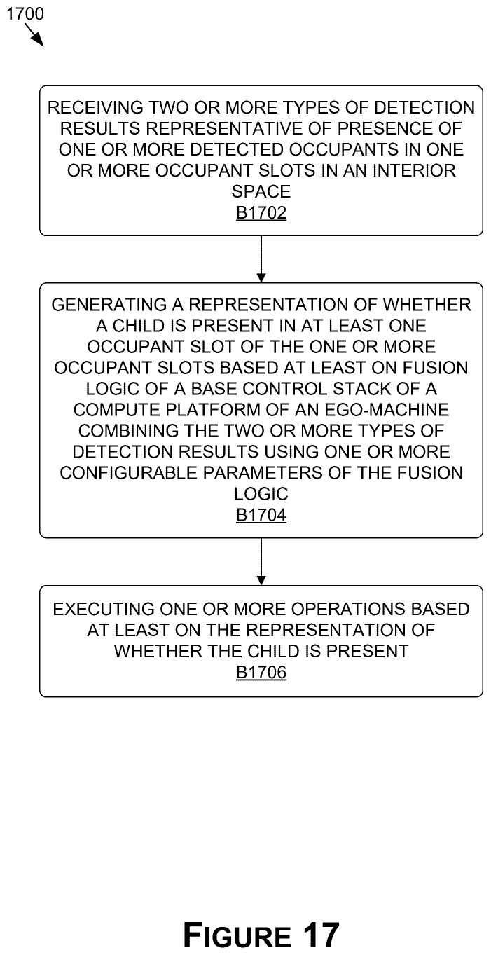

FIG. 17 is a flow diagram showing a method 1700 for child presence detection using fusion logic with one or more configurable parameters, in accordance with some embodiments of the present disclosure. The method 1700, at block B1702, includes receiving two or more types of detection results representative of presence of one or more detected occupants in one or more occupant slots in an interior space. For example, with respect to FIG. 1, depending on the detection task and/or the implementation, different types of sensor(s), sensor data 120a-n, and/or detection pipeline(s) 130a-n may be involved. In some embodiments, the detection pipeline(s) 130a-n may use any known technique or other techniques described herein to detect RADAR blockage, to detect the presence of a live occupant in an occupant slot (e.g., RADAR liveness detection, applying face or person detection to an image), to classify a detected occupant in an occupant slot as an adult or child (e.g., based on RADAR data, based on image data), to detect the presence of a human or pet in an occupant slot (e.g., based on RADAR data, based on image data), to detect a child seat (e.g., from an image of an occupant slot), to estimate or classify the age of an occupant (e.g., face-based age estimation, size-based age estimation), and/or otherwise, and the detection pipeline(s) 130a-n (or some other component of the base control stack 110) may provide the sensor fusion logic 140 with the corresponding detection results.

The method 1700, at block B1704, includes generating a representation of whether a child is present in at least one occupant slot of the one or more occupant slots based at least on fusion logic of a base control stack of a compute platform of an ego-machine combining the two or more types of detection results using one or more configurable parameters of the fusion logic. For example, with respect to FIG. 1, the sensor fusion logic 140 of the base control stack 110 may use one or more (e.g., slot-dependent) state machines and/or (e.g., configurable, preset) parameters to select, combine, weight, and/or apply threshold(s) to (e.g., binary) signals or other representations of detection results (e.g., which may be representative of predicted presence) generated by a corresponding detection pipeline(s) 130a-n (e.g., detection confidence(s)) to generate a unified representation of whether a child (or adult) was detected in an occupant slot (e.g., a unified confidence, a binary result). FIGS. 10-14 illustrate some examples of configurable sensor fusion for child presence detection which may be implemented in the sensor fusion logic 140 for one or more occupant slots.

The method 1700, at block B1706, includes executing one or more operations based at least on the representation of whether the child present. For example, with respect to FIG. 1, is the sensor fusion logic 140 may provide a representation of a unified result (e.g., a binary result, detection confidence) to corresponding control component(s) 190 or otherwise instruct the corresponding control component(s) 190 to take a corresponding action. Generally, the applicable control component(s) 190 may depend on the detection task and/or the implementation. Taking child presence detection as an example, the control component(s) 190 may determine that a unified result indicates a child is present in a particular occupant slot (e.g., a vehicle seat, a footwell). In some embodiments, based on detecting a child in a particular vehicle seat, the control component(s) 190 may trigger disabling of an airbag deployment for that seat and/or engage child lock settings. In some embodiments, based on determining that a child is present in any occupant slot, the control component(s) 190 may trigger a determination of whether the child is unattended using any known technique. The control component(s) 190 may determine whether at least one adult is present based on at least one occupant having a predicted age (e.g., estimated using any known technique) above a threshold, at least one occupant having a predicted height (e.g., a height derived from a size of one or more predicted limb lengths) above a threshold (e.g., greater than 50% the size of an average 10 year old), and/or otherwise. In some embodiments, based on determining that a detected child is unattended, the control component(s) 190 may trigger one or more actions (e.g., trigger an audible or visual alert, control temperature, unlock door(s), trigger or enable one or more camera feeds, etc.).

The systems and methods described herein may be used by—or may be used in combination with—without limitation, non-autonomous vehicles or machines, semi-autonomous vehicles or machines (e.g., in one or more adaptive driver assistance systems (ADAS)), piloted and un-piloted robots or robotic platforms, warehouse vehicles, off-road vehicles, vehicles coupled to one or more trailers, flying vessels, boats, shuttles, emergency response vehicles, motorcycles, electric or motorized bicycles, aircraft, construction vehicles, trains, underwater craft, remotely operated vehicles such as drones, and/or other vehicle types. Further, the systems and methods described herein may be used for a variety of purposes, by way of example and without limitation, for machine control, machine locomotion, machine driving, synthetic data generation, model training, perception, augmented reality, virtual reality, mixed reality, robotics, security and surveillance, simulation and digital twinning, autonomous or semi-autonomous machine applications, deep learning, environment simulation, object or actor simulation and/or digital twinning, data center processing, conversational AI, light transport simulation (e.g., ray-tracing, path tracing, etc.), distributed or collaborative content creation for 3D assets (e.g., using universal scene descriptor (USD) data, such as OpenUSD, and/or other data types), cloud computing, generative artificial intelligence (e.g., using one or more diffusion models, transformer models, etc.), language model applications (e.g., large language models (LLMs), vision language models (VLMs), etc.), and/or any other suitable applications.

Disclosed embodiments may be comprised in a variety of different systems such as automotive systems (e.g., a control system for an autonomous or semi-autonomous machine, a perception system for an autonomous or semi-autonomous machine), systems implemented using a robot or robotic platform, aerial systems, medical systems, boating systems, smart area monitoring systems, systems for performing deep learning operations, systems for performing simulation operations (e.g., in a driving or vehicle simulation, in a robotics simulation, in a smart cities or surveillance simulation, etc.), systems for performing digital twin operations (e.g., in conjunction with a collaborative content creation platform or system, such as, without limitation, NVIDIA's OMNIVERSE and/or another platform, system, or service that uses USD or OpenUSD data types), systems implemented using an edge device, systems incorporating one or more virtual machines (VMs), systems for performing synthetic data generation operations (e.g., using one or more neural rendering fields (NERFs), gaussian splat techniques, diffusion models, transformer models, etc.), systems implemented at least partially in a data center, systems for performing conversational AI operations, systems implementing one or more language models - such as one or more large language models (LLMs), one or more vision language models (VLMs), one or more multi-modal language models, etc., systems for performing light transport simulation, systems for performing collaborative content creation for 3D assets (e.g., using universal scene descriptor (USD) data, such as OpenUSD, computer aided design (CAD) data, 2D and/or 3D graphics or design data, and/or other data types), systems implemented at least partially using cloud computing resources, and/or other types of systems.

In some examples, the machine learning model(s) (e.g., deep neural networks, language models, LLMs, VLMs, multi-modal language models, perception models, tracking models, fusion models, transformer models, diffusion models, encoder-only models, decoder-only models, encoder-decoder models, neural rendering field (NERF) models, etc.) described herein may be packaged as a microservice—such an inference microservice (e.g., NVIDIA NIMs)—which may include a container (e.g., an operating system (OS)-level virtualization package) that may include an application programming interface (API) layer, a server layer, a runtime layer, and/or at least one model “engine.” For example, the inference microservice may include the container itself and the model(s) (e.g., weights and biases). In some instances, such as where the machine learning model(s) is small enough (e.g., has a small enough number of parameters), the model(s) may be included within the container itself. In other examples - such as where the model(s) is large - the model(s) may be hosted/stored in the cloud (e.g., in a data center) and/or may be hosted on-premises and/or at the edge (e.g., on a local server or computing device, but outside of the container). In such embodiments, the model(s) may be accessible via one or more APIs-such as REST APIs. As such, and in some embodiments, the machine learning model(s) described herein may be deployed as an inference microservice to accelerate deployment of a model(s) on any cloud, data center, or edge computing system, while ensuring the data is secure. For example, the inference microservice may include one or more APIs, a pre-configured container for simplified deployment, an optimized inference engine (e.g., built using a standardized AI model deployment an execution software, such as NVIDIA's Triton Inference Server, and/or one or more APIs for high performance deep learning inference, which may include an inference runtime and model optimizations that deliver low latency and high throughput for production applications—such as NVIDIA's Tensor®), and/or enterprise management data for telemetry (e.g., including identity, metrics, health checks, and/or monitoring).

The machine learning model(s) described herein may be included as part of the microservice along with an accelerated infrastructure with the ability to deploy with a single command and/or orchestrate and auto-scale with a container orchestration system on accelerated infrastructure (e.g., on a single device up to data center scale). As such, the inference microservice may include the machine learning model(s) (e.g., that has been optimized for high performance inference), an inference runtime software to execute the machine learning model(s) and provide outputs/responses to inputs (e.g., user queries, prompts, etc.), and enterprise management software to provide health checks, identity, and/or other monitoring. In some embodiments, the inference microservice may include software to perform in-place replacement and/or updating to the machine learning model(s). When replacing or updating, the software that performs the replacement/updating may maintain user configurations of the inference runtime software and enterprise management software.

In some embodiments, the systems and methods described herein may be performed within a simulation environment (e.g., NVIDIA's DriveSIM) using simulated data (e.g., simulated sensor data of simulated sensors of a simulated machine). For example, simulated (or virtual) sensor data (e.g., images of a simulated environment such as highway or warehouse environment generated from the perspective of one or more simulated sensors of a simulated ego-machine) may be applied to one or more detection pipelines, (e.g., configurable) sensor logic may be used to combine the detection results (and/or signals corresponding to the simulated sensor data) and generate a unified representation of whether one or more conditions are present in the simulated environment, and the unified result may be used to control the simulated ego-machine within the simulated environment. These simulated operations may be used to test performance of the underlying algorithms, systems, and/or processes prior to deploying them in the real-world. In some instances, the simulation may be used to generate synthetic training data—e.g., images of a simulated environment generated from the perspective of one or more simulated sensors of a simulated ego-machine, and the synthetic training data (in addition or as an alternative to real-world data) may be used to train a multi-modal language model (e.g., a VLM). In any example, such as where a simulation environment is used for testing, validation, training, etc., the simulation environment and/or associated training data may be rendered or otherwise generated using one or more light transport algorithms—such as ray-tracing and/or path-tracing algorithms. In some embodiments, the simulation environment and/or one or more objects, features, or components thereof may be generated or managed within a three-dimensional (3D) content collaboration platform (e.g., NVIDIA's OMNIVERSE) for industrial digitalization, generative physical AI, and/or other use cases, applications, or services. For example, the content collaboration platform or system may include a system for using or developing universal scene descriptor (USD) (e.g., OpenUSD) data for managing objects, features, scenes, etc. within a simulated environment, digital environment, etc. The platform may include real physics simulation, such as using NVIDIA's PhysX SDK, in order to simulate real physics and physical interactions with simulations hosted by the platform. The platform may integrate OpenUSD along with ray tracing/path tracing/light transport simulation (e.g., NVIDIA's RTX rendering technologies) into software tools and simulation workflows for building, training, deploying, or testing AI systems—such as systems for testing, validating, training (e.g., machine learning models, neural networks, etc.), and/or other tasks related to automotive, robot, machine, or other applications.

### Example Autonomous Vehicle

FIG. 18A is an illustration of an example autonomous or semi-autonomous vehicle or machine 1800, in accordance with some embodiments of the present disclosure. The autonomous or semi-autonomous vehicle or machine 1800 (alternatively referred to herein as the “vehicle 1800,” “machine 1800,” “ego-vehicle 1800,” “ego-machine 1800,” “robot 1800,” etc.) may include, without limitation, a passenger vehicle, such as a car, a truck, a bus, a first responder vehicle, a shuttle, an electric or motorized bicycle, a motorcycle, a fire truck, a police vehicle, an ambulance, a boat, a construction vehicle, an underwater craft, a robotic vehicle, a drone, an airplane, a vehicle coupled to a trailer (e.g., a semi-tractor-trailer truck used for hauling cargo), and/or another type of vehicle (e.g., that is unmanned and/or that accommodates one or more passengers). Autonomous vehicles are generally described in terms of automation levels, defined by the National Highway Traffic Safety Administration (NHTSA), a division of the US Department of Transportation, and the Society of Automotive Engineers (SAE) “Taxonomy and Definitions for Terms Related to Driving Automation Systems for On-Road Motor Vehicles” (Standard No. J3016-201806, published on Jun. 15, 2018, Standard No. J3016-201609, published on Sep. 30, 2016, and previous and future versions of this standard). The vehicle 1800 may be capable of functionality in accordance with one or more of Level 3-Level 5 of the autonomous driving levels. The vehicle 1800 may be capable of functionality in accordance with one or more of Level 1-Level 5 of the autonomous driving levels. For example, the vehicle 1800 may be capable of driver assistance (Level 1), partial automation (Level 2), conditional automation (Level 3), high automation (Level 4), and/or full automation (Level 5), depending on the embodiment. The term “autonomous,” as used herein, may include any and/or all types of autonomy for the vehicle 1800 or other machine, such as being fully autonomous, being highly autonomous, being conditionally autonomous, being partially autonomous, providing assistive autonomy, being semi-autonomous, being primarily autonomous, or other designation.

The vehicle 1800 may include components such as a chassis, a vehicle body, wheels (e.g., 2, 4, 6, 8, 18, etc.), tires, axles, and other components of a vehicle. The vehicle 1800 may include a propulsion system 1850, such as an internal combustion engine, hybrid electric power plant, an all-electric engine, and/or another propulsion system type. The propulsion system 1850 may be connected to a drive train of the vehicle 1800, which may include a transmission, to allow the propulsion of the vehicle 1800. The propulsion system 1850 may be controlled in response to receiving signals from the throttle/accelerator 1852.

A steering system 1854, which may include a steering wheel, may be used to steer the vehicle 1800 (e.g., along a desired path or route) when the propulsion system 1850 is operating (e.g., when the vehicle is in motion). The steering system 1854 may receive signals from a steering actuator 1856. The steering wheel may be optional for full automation (Level 5) functionality.

The brake sensor system 1846 may be used to operate the vehicle brakes in response to receiving signals from the brake actuators 1848 and/or brake sensors.

Controller(s) 1836, which may include one or more system on chips (SoCs) 1804 (FIG. 18C) and/or GPU(s), may provide signals (e.g., representative of commands) to one or more components and/or systems of the vehicle 1800. For example, the controller(s) may send signals to operate the vehicle brakes via one or more brake actuators 1848, to operate the steering system 1854 via one or more steering actuators 1856, to operate the propulsion system 1850 via one or more throttle/accelerators 1852. The controller(s) 1836 may include one or more onboard (e.g., integrated) computing devices (e.g., supercomputers) that process sensor signals, and output operation commands (e.g., signals representing commands) to allow autonomous driving and/or to assist a human driver in driving the vehicle 1800. The controller(s) 1836 may include a first controller 1836 for autonomous driving functions, a second controller 1836 for functional safety functions, a third controller 1836 for artificial intelligence functionality (e.g., computer vision), a fourth controller 1836 for infotainment functionality, a fifth controller 1836 for redundancy in emergency conditions, and/or other controllers. In some examples, a single controller 1836 may handle two or more of the above functionalities, two or more controllers 1836 may handle a single functionality, and/or any combination thereof.

The controller(s) 1836 may provide the signals for controlling one or more components and/or systems of the vehicle 1800 in response to sensor data received from one or more sensors (e.g., sensor inputs). The sensor data may be received from, for example and without limitation, global navigation satellite systems (“GNSS”) sensor(s) 1858 (e.g., Global Positioning System sensor(s)), RADAR sensor(s) 1860, ultrasonic sensor(s) 1862, LiDAR sensor(s) 1864, inertial measurement unit (IMU) sensor(s) 1866 (e.g., accelerometer(s), gyroscope(s), magnetic compass(es), magnetometer(s), etc.), microphone(s) 1896, stereo camera(s) 1868, wide-view camera(s) 1870 (e.g., fisheye cameras), infrared camera(s) 1872, surround camera(s) 1874 (e.g., 360 degree cameras), long-range and/or mid-range camera(s) 1898, speed sensor(s) 1844 (e.g., for measuring the speed of the vehicle 1800), vibration sensor(s) 1842, steering sensor(s) 1840, brake sensor(s) (e.g., as part of the brake sensor system 1846), one or more occupant monitoring system (OMS) sensor(s) 1801 (e.g., one or more interior cameras), and/or other sensor types.

One or more of the controller(s) 1836 may receive inputs (e.g., represented by input data) from an instrument cluster 1832 of the vehicle 1800 and provide outputs (e.g., represented by output data, display data, etc.) via a human-machine interface (HMI) display 1834, an audible annunciator, a loudspeaker, and/or via other components of the vehicle 1800. The outputs may include information such as vehicle velocity, speed, time, map data (e.g., the High Definition (“HD”) map 1822 of FIG. 18C), location data (e.g., the vehicle's 1800 location, such as on a map), direction, location of other vehicles (e.g., an occupancy grid), information about objects and status of objects as perceived by the controller(s) 1836, etc. For example, the HMI display 1834 may display information about the presence of one or more objects (e.g., a street sign, caution sign, traffic light changing, etc.), and/or information about driving maneuvers the vehicle has made, is making, or will make (e.g., changing lanes now, taking exit 34B in two miles, etc.).

The vehicle 1800 further includes a network interface 1824 which may use one or more wireless antenna(s) 1826 and/or modem(s) to communicate over one or more networks. For example, the network interface 1824 may be capable of communication over Long-Term Evolution (“LTE”), Wideband Code Division Multiple Access (“WCDMA”), Universal Mobile Telecommunications System (“UMTS”), Global System for Mobile communication (“GSM”), IMT-CDMA Multi-Carrier (“CDMA2000”), etc. The wireless antenna(s) 1826 may also allow communication between objects in the environment (e.g., vehicles, mobile devices, etc.), using local area network(s), such as Bluetooth, Bluetooth Low Energy (“LE”), Z-Wave, ZigBee, etc., and/or low power wide-area network(s) (“LPWANs”), such as LoRaWAN, SigFox, etc.

FIG. 18B is an example of camera locations and fields of view for the example autonomous vehicle 1800 of FIG. 18A, in accordance with some embodiments of the present disclosure. The cameras and respective fields of view are one example embodiment and are not intended to be limiting. For example, additional and/or alternative cameras may be included and/or the cameras may be located at different locations on the vehicle 1800.

The camera types for the cameras may include, but are not limited to, digital cameras that may be adapted for use with the components and/or systems of the vehicle 1800. The camera(s) may operate at automotive safety integrity level (ASIL) B and/or at another ASIL. The camera types may be capable of any image capture rate, such as 60 frames per second (fps), 120 fps, 240 fps, etc., depending on the embodiment. The cameras may be capable of using rolling shutters, global shutters, another type of shutter, or a combination thereof. In some examples, the color filter array may include a red clear clear clear (RCCC) color filter array, a red clear clear blue (RCCB) color filter array, a red blue green clear (RBGC) color filter array, a Foveon X3 color filter array, a Bayer sensors (RGGB) color filter array, a monochrome sensor color filter array, and/or another type of color filter array. In some embodiments, clear pixel cameras, such as cameras with an RCCC, an RCCB, and/or an RBGC color filter array, may be used in an effort to increase light sensitivity.

In some examples, one or more of the camera(s) may be used to perform advanced driver assistance systems (ADAS) functions (e.g., as part of a redundant or fail-safe design). For example, a Multi-Function Mono Camera may be installed to provide functions including lane departure warning, traffic sign assist and intelligent headlamp control. One or more of the camera(s) (e.g., all of the cameras) may record and provide image data (e.g., video) simultaneously.

One or more of the cameras may be mounted in a mounting assembly, such as a custom designed (three dimensional (“3D”) printed) assembly, in order to cut out stray light and reflections from within the car (e.g., reflections from the dashboard reflected in the windshield mirrors) which may interfere with the camera's image data capture abilities. With reference to wing-mirror mounting assemblies, the wing-mirror assemblies may be custom 3D printed so that the camera mounting plate matches the shape of the wing-mirror. In some examples, the camera(s) may be integrated into the wing-mirror. For side-view cameras, the camera(s) may also be integrated within the four pillars at each corner of the cabin.

Cameras with a field of view that include portions of the environment in front of the vehicle 1800 (e.g., front-facing cameras) may be used for surround view, to help identify forward facing paths and obstacles, as well aid in, with the help of one or more controllers 1836 and/or control SoCs, providing information critical to generating an occupancy grid and/or determining the preferred vehicle paths. Front-facing cameras may be used to perform many of the same ADAS functions as LiDAR, including emergency braking, pedestrian detection, and collision avoidance. Front-facing cameras may also be used for ADAS functions and systems including Lane Departure Warnings (“LDW”), Autonomous Cruise Control (“ACC”), and/or other functions such as traffic sign recognition.

A variety of cameras may be used in a front-facing configuration, including, for example, a monocular camera platform that includes a complementary metal oxide semiconductor (“CMOS”) color imager. Another example may be a wide-view camera(s) 1870 that may be used to perceive objects coming into view from the periphery (e.g., pedestrians, crossing traffic or bicycles). Although only one wide-view camera is illustrated in FIG. 18B, there may be any number (including zero) of wide-view cameras 1870 on the vehicle 1800. In addition, any number of long-range camera(s) 1898 (e.g., a long-view stereo camera pair) may be used for depth-based object detection, especially for objects for which a neural network has not yet been trained. The long-range camera(s) 1898 may also be used for object detection and classification, as well as basic object tracking.

Any number of stereo cameras 1868 may also be included in a front-facing configuration. In at least one embodiment, one or more of stereo camera(s) 1868 may include an integrated control unit comprising a scalable processing unit, which may provide a programmable logic (“FPGA”) and a multi-core micro-processor with an integrated Controller Area Network (“CAN”) or Ethernet interface on a single chip. Such a unit may be used to generate a 3D map of the vehicle's environment, including a distance estimate for all the points in the image. An alternative stereo camera(s) 1868 may include a compact stereo vision sensor(s) that may include two camera lenses (one each on the left and right) and an image processing chip that may measure the distance from the vehicle to the target object and use the generated information (e.g., metadata) to activate the autonomous emergency braking and lane departure warning functions. Other types of stereo camera(s) 1868 may be used in addition to, or alternatively from, those described herein.

Cameras with a field of view that include portions of the environment to the side of the vehicle 1800 (e.g., side-view cameras) may be used for surround view, providing information used to create and update the occupancy grid, as well as to generate side impact collision warnings. For example, surround camera(s) 1874 (e.g., four surround cameras 1874 as illustrated in FIG. 18B) may be positioned to on the vehicle 1800. The surround camera(s) 1874 may include wide-view camera(s) 1870, fisheye camera(s), 360 degree camera(s), and/or the like. Four example, four fisheye cameras may be positioned on the vehicle's front, rear, and sides. In an alternative arrangement, the vehicle may use three surround camera(s) 1874 (e.g., left, right, and rear), and may leverage one or more other camera(s) (e.g., a forward-facing camera) as a fourth surround view camera.

Cameras with a field of view that include portions of the environment to the rear of the vehicle 1800 (e.g., rear-view cameras) may be used for park assistance, surround view, rear collision warnings, and creating and updating the occupancy grid. A wide variety of cameras may be used including, but not limited to, cameras that are also suitable as a front-facing camera(s) (e.g., long-range and/or mid-range camera(s) 1898, stereo camera(s) 1868), infrared camera(s) 1872, etc.), as described herein.

Cameras with a field of view that include portions of the interior environment within the cabin of the vehicle 1800 (e.g., one or more OMS sensor(s) 1801) may be used as part of an occupant monitoring system (OMS) such as, but not limited to, a driver monitoring system (DMS). For example, OMS sensors (e.g., the OMS sensor(s) 1801) may be used (e.g., by the controller(s) 1836) to track an occupant's and/or driver's gaze direction, head pose, and/or blinking. This gaze information may be used to determine a level of attentiveness of the occupant or driver (e.g., to detect drowsiness, fatigue, and/or distraction), and/or to take responsive action to prevent harm to the occupant or operator. In some embodiments, data from OMS sensors may be used to allow gaze-controlled operations triggered by driver and/or non-driver occupants such as, but not limited to, adjusting cabin temperature and/or airflow, opening and closing windows, controlling cabin lighting, controlling entertainment systems, adjusting mirrors, adjusting seat positions, and/or other operations. In some embodiments, an OMS may be used for applications such as determining when objects and/or occupants have been left behind in a vehicle cabin (e.g., by detecting occupant presence after the driver exits the vehicle).

FIG. 18C is a block diagram of an example system architecture for the example autonomous vehicle 1800 of FIG. 18A, in accordance with some embodiments of the present disclosure. It should be understood that this and other arrangements described herein are set forth only as examples. Other arrangements and elements (e.g., machines, interfaces, functions, orders, groupings of functions, etc.) may be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by entities may be carried out by hardware, firmware, and/or software. For instance, various functions may be carried out by a processor executing instructions stored in memory.

Each of the components, features, and systems of the vehicle 1800 in FIG. 18C are illustrated as being connected via bus 1802. The bus 1802 may include a Controller Area Network (CAN) data interface (alternatively referred to herein as a “CAN bus”). A CAN may be a network inside the vehicle 1800 used to aid in control of various features and functionality of the vehicle 1800, such as actuation of brakes, acceleration, braking, steering, windshield wipers, etc. A CAN bus may be configured to have dozens or even hundreds of nodes, each with its own unique identifier (e.g., a CAN ID). The CAN bus may be read to find steering wheel angle, ground speed, engine revolutions per minute (RPMs), button positions, and/or other vehicle status indicators. The CAN bus may be ASIL B compliant.

Although the bus 1802 is described herein as being a CAN bus, this is not intended to be limiting. For example, in addition to, or alternatively from, the CAN bus, FlexRay and/or Ethernet may be used. Additionally, although a single line is used to represent the bus 1802, this is not intended to be limiting. For example, there may be any number of busses 1802, which may include one or more CAN busses, one or more FlexRay busses, one or more Ethernet busses, and/or one or more other types of busses using a different protocol. In some examples, two or more busses 1802 may be used to perform different functions, and/or may be used for redundancy. For example, a first bus 1802 may be used for collision avoidance functionality and a second bus 1802 may be used for actuation control. In any example, each bus 1802 may communicate with any of the components of the vehicle 1800, and two or more busses 1802 may communicate with the same components. In some examples, each SoC 1804, each controller 1836, and/or each computer within the vehicle may have access to the same input data (e.g., inputs from sensors of the vehicle 1800), and may be connected to a common bus, such the CAN bus.

The vehicle 1800 may include one or more controller(s) 1836, such as those described herein with respect to FIG. 18A. The controller(s) 1836 may be used for a variety of functions. The controller(s) 1836 may be coupled to any of the various other components and systems of the vehicle 1800, and may be used for control of the vehicle 1800, artificial intelligence of the vehicle 1800, infotainment for the vehicle 1800, and/or the like.

The vehicle 1800 may include a system(s) on a chip (SoC) 1804. The SoC 1804 may include CPU(s) 1806, GPU(s) 1808, processor(s) 1810, cache(s) 1812, accelerator(s) 1814, data store(s) 1816, and/or other components and features not illustrated. The SoC(s) 1804 may be used to control the vehicle 1800 in a variety of platforms and systems. For example, the SoC(s) 1804 may be combined in a system (e.g., the system of the vehicle 1800) with an HD map 1822 which may obtain map refreshes and/or updates via a network interface 1824 from one or more servers (e.g., server(s) 1878 of FIG. 18D).

The CPU(s) 1806 may include a CPU cluster or CPU complex (alternatively referred to herein as a “CCPLEX”). The CPU(s) 1806 may include multiple cores and/or L2 caches. For example, in some embodiments, the CPU(s) 1806 may include eight cores in a coherent multi-processor configuration. In some embodiments, the CPU(s) 1806 may include four dual-core clusters where each cluster has a dedicated L2 cache (e.g., a 2 MB L2 cache). The CPU(s) 1806 (e.g., the CCPLEX) may be configured to support simultaneous cluster operation allowing any combination of the clusters of the CPU(s) 1806 to be active at any given time.

The CPU(s) 1806 may implement power management capabilities that include one or more of the following features: individual hardware blocks may be clock-gated automatically when idle to save dynamic power; each core clock may be gated when the core is not actively executing instructions due to execution of WFI/WFE instructions; each core may be independently power-gated; each core cluster may be independently clock-gated when all cores are clock-gated or power-gated; and/or each core cluster may be independently power-gated when all cores are power-gated. The CPU(s) 1806 may further implement an enhanced algorithm for managing power states, where allowed power states and expected wakeup times are specified, and the hardware/microcode determines the best power state to enter for the core, cluster, and CCPLEX. The processing cores may support simplified power state entry sequences in software with the work offloaded to microcode.

The GPU(s) 1808 may include an integrated GPU (alternatively referred to herein as an “iGPU”). The GPU(s) 1808 may be programmable and may be efficient for parallel workloads. The GPU(s) 1808, in some examples, may use an enhanced tensor instruction set. The GPU(s) 1808 may include one or more streaming microprocessors, where each streaming microprocessor may include an L1 cache (e.g., an L1 cache with at least 96 KB storage capacity), and two or more of the streaming microprocessors may share an L2 cache (e.g., an L2 cache with a 512 KB storage capacity). In some embodiments, the GPU(s) 1808 may include at least eight streaming microprocessors. The GPU(s) 1808 may use compute application programming interface(s) (API(s)). In addition, the GPU(s) 1808 may use one or more parallel computing platforms and/or programming models (e.g., NVIDIA's CUDA).

The GPU(s) 1808 may be power-optimized for best performance in automotive and embedded use cases. For example, the GPU(s) 1808 may be fabricated on a Fin field-effect transistor (FinFET). However, this is not intended to be limiting and the GPU(s) 1808 may be fabricated using other semiconductor manufacturing processes. Each streaming microprocessor may incorporate a number of mixed-precision processing cores partitioned into multiple blocks. For example, and without limitation, 64 PF32 cores and 32 PF 64 cores may be partitioned into four processing blocks. In such an example, each processing block may be allocated 16 FP32 cores, 8 FP64 cores, 16 INT32 cores, two mixed-precision NVIDIA TENSOR COREs for deep learning matrix arithmetic, an L0 instruction cache, a warp scheduler, a dispatch unit, and/or a 64 KB register file. In addition, the streaming microprocessors may include independent parallel integer and floating-point data paths to provide for efficient execution of workloads with a mix of computation and addressing calculations. The streaming microprocessors may include independent thread scheduling capability to allow finer-grain synchronization and cooperation between parallel threads. The streaming microprocessors may include a combined L1 data cache and shared memory unit in order to improve performance while simplifying programming.

The GPU(s) 1808 may include a high bandwidth memory (HBM) and/or a 16 GB HBM2 memory subsystem to provide, in some examples, about 900 GB/second peak memory bandwidth. In some examples, in addition to, or alternatively from, the HBM memory, a synchronous graphics random-access memory (SGRAM) may be used, such as a graphics double data rate type five synchronous random-access memory (GDDR5).

The GPU(s) 1808 may include unified memory technology including access counters to allow for more accurate migration of memory pages to the processor that accesses them most frequently, thereby improving efficiency for memory ranges shared between processors. In some examples, address translation services (ATS) support may be used to allow the GPU(s) 1808 to access the CPU(s) 1806 page tables directly. In such examples, when the GPU(s) 1808 memory management unit (MMU) experiences a miss, an address translation request may be transmitted to the CPU(s) 1806. In response, the CPU(s) 1806 may look in its page tables for the virtual-to-physical mapping for the address and transmits the translation back to the GPU(s) 1808. As such, unified memory technology may allow a single unified virtual address space for memory of both the CPU(s) 1806 and the GPU(s) 1808, thereby simplifying the GPU(s) 1808 programming and porting of applications to the GPU(s) 1808.

In addition, the GPU(s) 1808 may include an access counter that may keep track of the frequency of access of the GPU(s) 1808 to memory of other processors. The access counter may help ensure that memory pages are moved to the physical memory of the processor that is accessing the pages most frequently.

The SoC(s) 1804 may include any number of cache(s) 1812, including those described herein. For example, the cache(s) 1812 may include an L3 cache that is available to both the CPU(s) 1806 and the GPU(s) 1808 (e.g., that is connected both the CPU(s) 1806 and the GPU(s) 1808). The cache(s) 1812 may include a write-back cache that may keep track of states of lines, such as by using a cache coherence protocol (e.g., MEI, MESI, MSI, etc.). The L3 cache may include 4 MB or more, depending on the embodiment, although smaller cache sizes may be used.

The SoC(s) 1804 may include an arithmetic logic unit(s) (ALU(s)) which may be leveraged in performing processing with respect to any of the variety of tasks or operations of the vehicle 1800—such as processing DNNs. In addition, the SoC(s) 1804 may include a floating point unit(s) (FPU(s))—or other math coprocessor or numeric coprocessor types—for performing mathematical operations within the system. For example, the SoC(s) 1804 may include one or more FPUs integrated as execution units within a CPU(s) 1806 and/or GPU(s) 1808.

The SoC(s) 1804 may include one or more accelerators 1814 (e.g., hardware accelerators, software accelerators, or a combination thereof). For example, the SoC(s) 1804 may include a hardware acceleration cluster that may include optimized hardware accelerators and/or large on-chip memory. The large on-chip memory (e.g., 4 MB of SRAM), may allow the hardware acceleration cluster to accelerate neural networks and other calculations. The hardware acceleration cluster may be used to complement the GPU(s) 1808 and to off-load some of the tasks of the GPU(s) 1808 (e.g., to free up more cycles of the GPU(s) 1808 for performing other tasks). As an example, the accelerator(s) 1814 may be used for targeted workloads (e.g., perception, convolutional neural networks (CNNs), etc.) that are stable enough to be amenable to acceleration. The term “CNN,” as used herein, may include all types of CNNs, including region-based or regional convolutional neural networks (RCNNs) and Fast RCNNs (e.g., as used for object detection).

The accelerator(s) 1814 (e.g., the hardware acceleration cluster) may include a deep learning accelerator(s) (DLA). The DLA(s) may include one or more Tensor processing units (TPUs) that may be configured to provide an additional ten trillion operations per second for deep learning applications and inferencing. The TPUs may be accelerators configured to, and optimized for, performing image processing functions (e.g., for CNNs, RCNNs, etc.). The DLA(s) may further be optimized for a specific set of neural network types and floating point operations, as well as inferencing. The design of the DLA(s) may provide more performance per millimeter than a general-purpose GPU, and vastly exceeds the performance of a CPU. The TPU(s) may perform several functions, including a single-instance convolution function, supporting, for example, INT8, INT16, and FP16 data types for both features and weights, as well as post-processor functions.

The DLA(s) may quickly and efficiently execute neural networks, especially CNNs, on processed or unprocessed data for any of a variety of functions, including, for example and without limitation: a CNN for object identification and detection using data from camera sensors; a CNN for distance estimation using data from camera sensors; a CNN for emergency vehicle detection and identification and detection using data from microphones; a CNN for facial recognition and vehicle owner identification using data from camera sensors; and/or a CNN for security and/or safety related events.

The DLA(s) may perform any function of the GPU(s) 1808, and by using an inference accelerator, for example, a designer may target either the DLA(s) or the GPU(s) 1808 for any function. For example, the designer may focus processing of CNNs and floating point operations on the DLA(s) and leave other functions to the GPU(s) 1808 and/or other accelerator(s) 1814.

The accelerator(s) 1814 (e.g., the hardware acceleration cluster) may include a programmable vision accelerator(s) (PVA), which may alternatively be referred to herein as a computer vision accelerator. The PVA(s) may be designed and configured to accelerate computer vision algorithms for the advanced driver assistance systems (ADAS), autonomous driving, and/or augmented reality (AR) and/or virtual reality (VR) applications. The PVA(s) may provide a balance between performance and flexibility. For example, each PVA(s) may include, for example and without limitation, any number of reduced instruction set computer (RISC) cores, direct memory access (DMA), and/or any number of vector processors.

The RISC cores may interact with image sensors (e.g., the image sensors of any of the cameras described herein), image signal processor(s), and/or the like. Each of the RISC cores may include any amount of memory. The RISC cores may use any of a number of protocols, depending on the embodiment. In some examples, the RISC cores may execute a real-time operating system (RTOS). The RISC cores may be implemented using one or more integrated circuit devices, application specific integrated circuits (ASICs), and/or memory devices. For example, the RISC cores may include an instruction cache and/or a tightly coupled RAM.

The DMA may allow components of the PVA(s) to access the system memory independently of the CPU(s) 1806. The DMA may support any number of features used to provide optimization to the PVA including, but not limited to, supporting multi-dimensional addressing and/or circular addressing. In some examples, the DMA may support up to six or more dimensions of addressing, which may include block width, block height, block depth, horizontal block stepping, vertical block stepping, and/or depth stepping.

The vector processors may be programmable processors that may be designed to efficiently and flexibly execute programming for computer vision algorithms and provide signal processing capabilities. In some examples, the PVA may include a PVA core and two vector processing subsystem partitions. The PVA core may include a processor subsystem, DMA engine(s) (e.g., two DMA engines), and/or other peripherals. The vector processing subsystem may operate as the primary processing engine of the PVA, and may include a vector processing unit (VPU), an instruction cache, and/or vector memory (e.g., VMEM). A VPU core may include a digital signal processor such as, for example, a single instruction, multiple data (SIMD), very long instruction word (VLIW) digital signal processor. The combination of the SIMD and VLIW may enhance throughput and speed.

Each of the vector processors may include an instruction cache and may be coupled to dedicated memory. As a result, in some examples, each of the vector processors may be configured to execute independently of the other vector processors. In other examples, the vector processors that are included in a particular PVA may be configured to employ data parallelism. For example, in some embodiments, the plurality of vector processors included in a single PVA may execute the same computer vision algorithm, but on different regions of an image. In other examples, the vector processors included in a particular PVA may simultaneously execute different computer vision algorithms, on the same image, or even execute different algorithms on sequential images or portions of an image. Among other things, any number of PVAs may be included in the hardware acceleration cluster and any number of vector processors may be included in each of the PVAs. In addition, the PVA(s) may include additional error correcting code (ECC) memory, to enhance overall system safety.

The accelerator(s) 1814 (e.g., the hardware acceleration cluster) may include a computer vision network on-chip and SRAM, for providing a high-bandwidth, low latency SRAM for the accelerator(s) 1814. In some examples, the on-chip memory may include at least 4 MB SRAM, consisting of, for example and without limitation, eight field-configurable memory blocks, that may be accessible by both the PVA and the DLA. Each pair of memory blocks may include an advanced peripheral bus (APB) interface, configuration circuitry, a controller, and a multiplexer. Any type of memory may be used. The PVA and DLA may access the memory via a backbone that provides the PVA and DLA with high-speed access to memory. The backbone may include a computer vision network on-chip that interconnects the PVA and the DLA to the memory (e.g., using the APB).

The computer vision network on-chip may include an interface that determines, before transmission of any control signal/address/data, that both the PVA and the DLA provide ready and valid signals. Such an interface may provide for separate phases and separate channels for transmitting control signals/addresses/data, as well as burst-type communications for continuous data transfer. This type of interface may comply with ISO 26262 or IEC 61508 standards, although other standards and protocols may be used.

In some examples, the SoC(s) 1804 may include a real-time ray-tracing hardware accelerator, such as described in U.S. Pat. No. 10,885,698, issued on Jan. 5, 2021. The real-time ray-tracing hardware accelerator may be used to quickly and efficiently determine the positions and extents of objects (e.g., within a world model), to generate real-time visualization simulations, for RADAR signal interpretation, for sound propagation synthesis and/or analysis, for simulation of SONAR systems, for general wave propagation simulation, for comparison to LiDAR data for purposes of localization and/or other functions, and/or for other uses. In some embodiments, one or more tree traversal units (TTUs) may be used for executing one or more ray-tracing related operations.

The accelerator(s) 1814 (e.g., the hardware accelerator cluster) have a wide array of uses for autonomous driving. The PVA may be a programmable vision accelerator that may be used for key processing stages in ADAS and autonomous vehicles. The PVA's capabilities are a good match for algorithmic domains needing predictable processing, at low power and low latency. As such, the PVA performs well on semi-dense or dense regular computation, even on small data sets, which need predictable run-times with low latency and low power. Thus, in the context of platforms for autonomous vehicles, the PVAs are designed to run classic computer vision algorithms, as they are efficient at object detection and operating on integer math.

For example, according to one embodiment of the technology, the PVA is used to perform computer stereo vision. A semi-global matching-based algorithm may be used in some examples, although this is not intended to be limiting. Many applications for Level 3-5 autonomous driving require motion estimation/stereo matching on-the-fly (e.g., structure from motion, pedestrian recognition, lane detection, etc.). The PVA may perform computer stereo vision function on inputs from two monocular cameras.

In some examples, the PVA may be used to perform dense optical flow. According to process raw RADAR data (e.g., using a 4D Fast Fourier Transform) to provide Processed RADAR. In other examples, the PVA is used for time of flight depth processing, by processing raw time of flight data to provide processed time of flight data, for example.

The DLA may be used to run any type of network to enhance control and driving safety, including for example, a neural network that outputs a measure of confidence for each object detection. Such a confidence value may be interpreted as a probability, or as providing a relative “weight” of each detection compared to other detections. This confidence value enables the system to make further decisions regarding which detections should be considered as true positive detections rather than false positive detections. For example, the system may set a threshold value for the confidence and consider only the detections exceeding the threshold value as true positive detections. In an automatic emergency braking (AEB) system, false positive detections would cause the vehicle to automatically perform emergency braking, which is obviously undesirable. Therefore, only the most confident detections should be considered as triggers for AEB. The DLA may run a neural network for regressing the confidence value. The neural network may take as its input at least some subset of parameters, such as bounding box dimensions, ground plane estimate obtained (e.g. from another subsystem), inertial measurement unit (IMU) sensor 1866 output that correlates with the vehicle 1800 orientation, distance, 3D location estimates of the object obtained from the neural network and/or other sensors (e.g., LiDAR sensor(s) 1864 or RADAR sensor(s) 1860), among others.

The SoC(s) 1804 may include data store(s) 1816 (e.g., memory). The data store(s) 1816 may be on-chip memory of the SoC(s) 1804, which may store neural networks to be executed on the GPU and/or the DLA. In some examples, the data store(s) 1816 may be large enough in capacity to store multiple instances of neural networks for redundancy and safety. The data store(s) 1816 may comprise L2 or L3 cache(s) 1812. Reference to the data store(s) 1816 may include reference to the memory associated with the PVA, DLA, and/or other accelerator(s) 1814, as described herein.

The SoC(s) 1804 may include one or more processor(s) 1810 (e.g., embedded processors). The processor(s) 1810 may include a boot and power management processor that may be a dedicated processor and subsystem to handle boot power and management functions and related security enforcement. The boot and power management processor may be a part of the SoC(s) 1804 boot sequence and may provide runtime power management services. The boot power and management processor may provide clock and voltage programming, assistance in system low power state transitions, management of SoC(s) 1804 thermals and temperature sensors, and/or management of the SoC(s) 1804 power states. Each temperature sensor may be implemented as a ring-oscillator whose output frequency is proportional to temperature, and the SoC(s) 1804 may use the ring-oscillators to detect temperatures of the CPU(s) 1806, GPU(s) 1808, and/or accelerator(s) 1814. If temperatures are determined to exceed a threshold, the boot and power management processor may enter a temperature fault routine and put the SoC(s) 1804 into a lower power state and/or put the vehicle 1800 into a chauffeur to safe stop mode (e.g., bring the vehicle 1800 to a safe stop).

The processor(s) 1810 may further include a set of embedded processors that may serve as an audio processing engine. The audio processing engine may be an audio subsystem that enables full hardware support for multi-channel audio over multiple interfaces, and a broad and flexible range of audio I/O interfaces. In some examples, the audio processing engine is a dedicated processor core with a digital signal processor with dedicated RAM.

The processor(s) 1810 may further include an always on processor engine that may provide necessary hardware features to support low power sensor management and wake use cases. The always on processor engine may include a processor core, a tightly coupled RAM, supporting peripherals (e.g., timers and interrupt controllers), various I/O controller peripherals, and routing logic.

The processor(s) 1810 may further include a safety cluster engine that includes a dedicated processor subsystem to handle safety management for automotive applications. The safety cluster engine may include two or more processor cores, a tightly coupled RAM, support peripherals (e.g., timers, an interrupt controller, etc.), and/or routing logic. In a safety mode, the two or more cores may operate in a lockstep mode and function as a single core with comparison logic to detect any differences between their operations.

The processor(s) 1810 may further include a real-time camera engine that may include a dedicated processor subsystem for handling real-time camera management.

The processor(s) 1810 may further include a high-dynamic range signal processor that may include an image signal processor that is a hardware engine that is part of the camera processing pipeline.

The processor(s) 1810 may include a video image compositor that may be a processing block (e.g., implemented on a microprocessor) that implements video post-processing functions needed by a video playback application to produce the final image for the player window. The video image compositor may perform lens distortion correction on wide-view camera(s) 1870, surround camera(s) 1874, and/or on in-cabin monitoring camera sensors. In-cabin monitoring camera sensor is preferably monitored by a neural network running on another instance of the Advanced SoC, configured to identify in cabin events and respond accordingly. An in-cabin system may perform lip reading to activate cellular service and place a phone call, dictate emails, change the vehicle's destination, activate or change the vehicle's infotainment system and settings, or provide voice-activated web surfing. Certain functions are available to the driver only when the vehicle is operating in an autonomous mode, and are disabled otherwise.

The video image compositor may include enhanced temporal noise reduction for both spatial and temporal noise reduction. For example, where motion occurs in a video, the noise reduction weights spatial information appropriately, decreasing the weight of information provided by adjacent frames. Where an image or portion of an image does not include motion, the temporal noise reduction performed by the video image compositor may use information from the previous image to reduce noise in the current image.

The video image compositor may also be configured to perform stereo rectification on input stereo lens frames. The video image compositor may further be used for user interface composition when the operating system desktop is in use, and the GPU(s) 1808 is not required to continuously render new surfaces. Even when the GPU(s) 1808 is powered on and active doing 3D rendering, the video image compositor may be used to offload the GPU(s) 1808 to improve performance and responsiveness.

The SoC(s) 1804 may further include a mobile industry processor interface (MIPI) camera serial interface for receiving video and input from cameras, a high-speed interface, and/or a video input block that may be used for camera and related pixel input functions. The SoC(s) 1804 may further include an input/output controller(s) that may be controlled by software and may be used for receiving I/O signals that are uncommitted to a specific role.

The SoC(s) 1804 may further include a broad range of peripheral interfaces to allow communication with peripherals, audio codecs, power management, and/or other devices. The SoC(s) 1804 may be used to process data from cameras (e.g., connected over Gigabit Multimedia Serial Link and Ethernet), sensors (e.g., LiDAR sensor(s) 1864, RADAR sensor(s) 1860, etc. that may be connected over Ethernet), data from bus 1802 (e.g., speed of vehicle 1800, steering wheel position, etc.), data from GNSS sensor(s) 1858 (e.g., connected over Ethernet or CAN bus). The SoC(s) 1804 may further include dedicated high-performance mass storage controllers that may include their own DMA engines, and that may be used to free the CPU(s) 1806 from routine data management tasks.

The SoC(s) 1804 may be an end-to-end platform with a flexible architecture that spans automation levels 3-5, thereby providing a comprehensive functional safety architecture that leverages and makes efficient use of computer vision and ADAS techniques for diversity and redundancy, provides a platform for a flexible, reliable driving software stack, along with deep learning tools. The SoC(s) 1804 may be faster, more reliable, and even more energy-efficient and space-efficient than conventional systems. For example, the accelerator(s) 1814, when combined with the CPU(s) 1806, the GPU(s) 1808, and the data store(s) 1816, may provide for a fast, efficient platform for level 3-5 autonomous vehicles.

The technology thus provides capabilities and functionality that cannot be achieved by conventional systems. For example, computer vision algorithms may be executed on CPUs, which may be configured using high-level programming language, such as the C programming language, to execute a wide variety of processing algorithms across a wide variety of visual data. However, CPUs are oftentimes unable to meet the performance requirements of many computer vision applications, such as those related to execution time and power consumption, for example. In particular, many CPUs are unable to execute complex object detection algorithms in real-time, which is a requirement of in-vehicle ADAS applications, and a requirement for practical Level 3-5 autonomous vehicles.

In contrast to conventional systems, by providing a CPU complex, GPU complex, and a hardware acceleration cluster, the technology described herein allows for multiple neural networks to be performed simultaneously and/or sequentially, and for the results to be combined together to allow Level 3-5 autonomous driving functionality. For example, a CNN executing on the DLA or dGPU (e.g., the GPU(s) 1820) may include a text and word recognition, allowing the supercomputer to read and understand traffic signs, including signs for which the neural network has not been specifically trained. The DLA may further include a neural network that is able to identify, interpret, and provides semantic understanding of the sign, and to pass that semantic understanding to the path planning modules running on the CPU Complex.

As another example, multiple neural networks may be run simultaneously, as is required for Level 3, 4, or 5 driving. For example, a warning sign consisting of “Caution: flashing lights indicate icy conditions,” along with an electric light, may be independently or collectively interpreted by several neural networks. The sign itself may be identified as a traffic sign by a first deployed neural network (e.g., a neural network that has been trained), the text “Flashing lights indicate icy conditions” may be interpreted by a second deployed neural network, which informs the vehicle's path planning software (preferably executing on the CPU Complex) that when flashing lights are detected, icy conditions exist. The flashing light may be identified by operating a third deployed neural network over multiple frames, informing the vehicle's path-planning software of the presence (or absence) of flashing lights. All three neural networks may run simultaneously, such as within the DLA and/or on the GPU(s) 1808.

In some examples, a CNN for facial recognition and vehicle owner identification may use data from camera sensors to identify the presence of an authorized driver and/or owner of the vehicle 1800. The always on sensor processing engine may be used to unlock the vehicle when the owner approaches the driver door and turn on the lights, and, in security mode, to disable the vehicle when the owner leaves the vehicle. In this way, the SoC(s) 1804 provide for security against theft and/or carjacking.

In another example, a CNN for emergency vehicle detection and identification may use data from microphones 1896 to detect and identify emergency vehicle sirens. In contrast to conventional systems, that use general classifiers to detect sirens and manually extract features, the SoC(s) 1804 use the CNN for classifying environmental and urban sounds, as well as classifying visual data. In a preferred embodiment, the CNN running on the DLA is trained to identify the relative closing speed of the emergency vehicle (e.g., by using the Doppler Effect). The CNN may also be trained to identify emergency vehicles specific to the local area in which the vehicle is operating, as identified by GNSS sensor(s) 1858. Thus, for example, when operating in Europe the CNN will seek to detect European sirens, and when in the United States the CNN will seek to identify only North American sirens. Once an emergency vehicle is detected, a control program may be used to execute an emergency vehicle safety routine, slowing the vehicle, pulling over to the side of the road, parking the vehicle, and/or idling the vehicle, with the assistance of ultrasonic sensors 1862, until the emergency vehicle(s) passes.

The vehicle may include a CPU(s) 1818 (e.g., discrete CPU(s), or dCPU(s)), that may be coupled to the SoC(s) 1804 via a high-speed interconnect (e.g., PCIe). The CPU(s) 1818 may include an X86 processor, for example. The CPU(s) 1818 may be used to perform any of a variety of functions, including arbitrating potentially inconsistent results between ADAS sensors and the SoC(s) 1804, and/or monitoring the status and health of the controller(s) 1836 and/or infotainment SoC 1830, for example.

The vehicle 1800 may include a GPU(s) 1820 (e.g., discrete GPU(s), or dGPU(s)), that may be coupled to the SoC(s) 1804 via a high-speed interconnect (e.g., NVIDIA's NVLINK). The GPU(s) 1820 may provide additional artificial intelligence functionality, such as by executing redundant and/or different neural networks, and may be used to train and/or update neural networks based on input (e.g., sensor data) from sensors of the vehicle 1800.

The vehicle 1800 may further include the network interface 1824 which may include one or more wireless antennas 1826 (e.g., one or more wireless antennas for different communication protocols, such as a cellular antenna, a Bluetooth antenna, etc.). The network interface 1824 may be used to allow wireless connectivity over the Internet with the cloud (e.g., with the server(s) 1878 and/or other network devices), with other vehicles, and/or with computing devices (e.g., client devices of passengers). To communicate with other vehicles, a direct link may be established between the two vehicles and/or an indirect link may be established (e.g., across networks and over the Internet). Direct links may be provided using a vehicle-to-vehicle communication link. The vehicle-to-vehicle communication link may provide the vehicle 1800 information about vehicles in proximity to the vehicle 1800 (e.g., vehicles in front of, on the side of, and/or behind the vehicle 1800). This functionality may be part of a cooperative adaptive cruise control functionality of the vehicle 1800.

The network interface 1824 may include a SoC that provides modulation and demodulation functionality and enables the controller(s) 1836 to communicate over wireless networks. The network interface 1824 may include a radio frequency front-end for up-conversion from baseband to radio frequency, and down conversion from radio frequency to baseband. The frequency conversions may be performed through well-known processes, and/or may be performed using super-heterodyne processes. In some examples, the radio frequency front end functionality may be provided by a separate chip. The network interface may include wireless functionality for communicating over LTE, WCDMA, UMTS, GSM, CDMA2000, Bluetooth, Bluetooth LE, Wi-Fi, Z-Wave, ZigBee, LoRaWAN, and/or other wireless protocols.

The vehicle 1800 may further include data store(s) 1828 which may include off-chip (e.g., off the SoC(s) 1804) storage. The data store(s) 1828 may include one or more storage elements including RAM, SRAM, DRAM, VRAM, Flash, hard disks, and/or other components and/or devices that may store at least one bit of data.

The vehicle 1800 may further include GNSS sensor(s) 1858. The GNSS sensor(s) 1858 (e.g., GPS, assisted GPS sensors, differential GPS (DGPS) sensors, etc.), to assist in mapping, perception, occupancy grid generation, and/or path planning functions. Any number of GNSS sensor(s) 1858 may be used, including, for example and without limitation, a GPS using a USB connector with an Ethernet to Serial (RS-232) bridge.

The vehicle 1800 may further include RADAR sensor(s) 1860. The RADAR sensor(s) 1860 may be used by the vehicle 1800 for long-range vehicle detection, even in darkness and/or severe weather conditions. RADAR functional safety levels may be ASIL B. The RADAR sensor(s) 1860 may use the CAN and/or the bus 1802 (e.g., to transmit data generated using the RADAR sensor(s) 1860) for control and to access object tracking data, with access to Ethernet to access raw data in some examples. A wide variety of RADAR sensor types may be used. For example, and without limitation, the RADAR sensor(s) 1860 may be suitable for front, rear, and side RADAR use. In some example, Pulse Doppler RADAR sensor(s) are used.

The RADAR sensor(s) 1860 may include different configurations, such as long range with narrow field of view, short range with wide field of view, short range side coverage, etc. In some examples, long-range RADAR may be used for adaptive cruise control functionality. The long-range RADAR systems may provide a broad field of view realized by two or more independent scans, such as within a 250 m range. The RADAR sensor(s) 1860 may help in distinguishing between static and moving objects, and may be used by ADAS systems for emergency brake assist and forward collision warning. Long-range RADAR sensors may include monostatic multimodal RADAR with multiple (e.g., six or more) fixed RADAR antennae and a high-speed CAN and FlexRay interface. In an example with six antennae, the central four antennae may create a focused beam pattern, designed to record the vehicle's 1800 surroundings at higher speeds with minimal interference from traffic in adjacent lanes. The other two antennae may expand the field of view, making it possible to quickly detect vehicles entering or leaving the vehicle's 1800 lane.

Mid-range RADAR systems may include, as an example, a range of up to 1860 m (front) or 80 m (rear), and a field of view of up to 42 degrees (front) or 1850 degrees (rear). Short-range RADAR systems may include, without limitation, RADAR sensors designed to be installed at both ends of the rear bumper. When installed at both ends of the rear bumper, such a RADAR sensor systems may create two beams that constantly monitor the blind spot in the rear and next to the vehicle.

Short-range RADAR systems may be used in an ADAS system for blind spot detection and/or lane change assist.

The vehicle 1800 may further include ultrasonic sensor(s) 1862. The ultrasonic sensor(s) 1862, which may be positioned at the front, back, and/or the sides of the vehicle 1800, may be used for park assist and/or to create and update an occupancy grid. A wide variety of ultrasonic sensor(s) 1862 may be used, and different ultrasonic sensor(s) 1862 may be used for different ranges of detection (e.g., 2.5 m, 4 m). The ultrasonic sensor(s) 1862 may operate at functional safety levels of ASIL B.

The vehicle 1800 may include LiDAR sensor(s) 1864. The LiDAR sensor(s) 1864 may be used for object and pedestrian detection, emergency braking, collision avoidance, and/or other functions. The LiDAR sensor(s) 1864 may be functional safety level ASIL B. In some examples, the vehicle 1800 may include multiple LiDAR sensors 1864 (e.g., two, four, six, etc.) that may use Ethernet (e.g., to provide data to a Gigabit Ethernet switch).

In some examples, the LiDAR sensor(s) 1864 may be capable of providing a list of objects and their distances for a 360-degree field of view. Commercially available LiDAR sensor(s) 1864 may have an advertised range of approximately 1800 m, with an accuracy of 2 cm-3 cm, and with support for a 1800 Mbps Ethernet connection, for example. In some examples, one or more non-protruding LiDAR sensors 1864 may be used. In such examples, the LiDAR sensor(s) 1864 may be implemented as a small device that may be embedded into the front, rear, sides, and/or corners of the vehicle 1800. The LiDAR sensor(s) 1864, in such examples, may provide up to a 120-degree horizontal and 35-degree vertical field-of-view, with a 200 m range even for low-reflectivity objects. Front-mounted LiDAR sensor(s) 1864 may be configured for a horizontal field of view between 45 degrees and 135 degrees. FIG. 18B illustrates example long-range and short-range horizontal fields-of-view for a LiDAR sensor 1864 with an example mounting location above the windshield, but other configurations such as those that include a grille-mounted LiDAR sensor 1864 (e.g., as illustrated in FIG. 18A) and/or a roof-mounted LiDAR scanner (e.g., for a data collection vehicle) are possible.

In some examples, LiDAR technologies, such as 3D flash LiDAR, may also be used. 3D Flash LiDAR uses a flash of a laser as a transmission source, to illuminate vehicle surroundings up to approximately 200 m. A flash LiDAR unit includes a receptor, which records the laser pulse transit time and the reflected light on each pixel, which in turn corresponds to the range from the vehicle to the objects. Flash LiDAR may allow for highly accurate and distortion-free images of the surroundings to be generated with every laser flash. In some examples, four flash LiDAR sensors may be deployed, one at each side of the vehicle 1800. Available 3D flash LiDAR systems include a solid-state 3D staring array LiDAR camera with no moving parts other than a fan (e.g., a non-scanning LiDAR device). The flash LiDAR device may use a 5 nanosecond class I (eye-safe) laser pulse per frame and may capture the reflected laser light in the form of 3D range point clouds and co-registered intensity data. By using flash LiDAR, and because flash LiDAR is a solid-state device with no moving parts, the LiDAR sensor(s) 1864 may be less susceptible to motion blur, vibration, and/or shock.

The vehicle may further include IMU sensor(s) 1866. The IMU sensor(s) 1866 may be located at a center of the rear axle of the vehicle 1800, in some examples. The IMU sensor(s) 1866 may include, for example and without limitation, an accelerometer(s), a magnetometer(s), a gyroscope(s), a magnetic compass(es), and/or other sensor types. In some examples, such as in six-axis applications, the IMU sensor(s) 1866 may include accelerometers and gyroscopes, while in nine-axis applications, the IMU sensor(s) 1866 may include accelerometers, gyroscopes, and magnetometers.

In some embodiments, the IMU sensor(s) 1866 may be implemented as a miniature, high performance GPS-Aided Inertial Navigation System (GPS/INS) that combines micro-electro-mechanical systems (MEMS) inertial sensors, a high-sensitivity GPS receiver, and advanced Kalman filtering algorithms to provide estimates of position, velocity, and attitude. As such, in some examples, the IMU sensor(s) 1866 may allow the vehicle 1800 to estimate heading without requiring input from a magnetic sensor by directly observing and correlating the changes in velocity from GPS to the IMU sensor(s) 1866. In some examples, the IMU sensor(s) 1866 and the GNSS sensor(s) 1858 may be combined in a single integrated unit.

The vehicle may include microphone(s) 1896 placed in and/or around the vehicle 1800. The microphone(s) 1896 may be used for emergency vehicle detection and identification, among other things.

The vehicle may further include any number of camera types, including stereo camera(s) 1868, wide-view camera(s) 1870, infrared camera(s) 1872, surround camera(s) 1874, long-range and/or mid-range camera(s) 1898, and/or other camera types. The cameras may be used to capture image data around an entire periphery of the vehicle 1800. The types of cameras used depends on the embodiments and requirements for the vehicle 1800, and any combination of camera types may be used to provide the necessary coverage around the vehicle 1800. In addition, the number of cameras may differ depending on the embodiment. For example, the vehicle may include six cameras, seven cameras, ten cameras, twelve cameras, and/or another number of cameras. The cameras may support, as an example and without limitation, Gigabit Multimedia Serial Link (GMSL) and/or Gigabit Ethernet. Each of the camera(s) is described with more detail herein with respect to FIG. 18A and FIG. 18B.

The vehicle 1800 may further include vibration sensor(s) 1842. The vibration sensor(s) 1842 may measure vibrations of components of the vehicle, such as the axle(s). For example, changes in vibrations may indicate a change in road surfaces. In another example, when two or more vibration sensors 1842 are used, the differences between the vibrations may be used to determine friction or slippage of the road surface (e.g., when the difference in vibration is between a power-driven axle and a freely rotating axle).

The vehicle 1800 may include an ADAS system 1838. The ADAS system 1838 may include a SoC, in some examples. The ADAS system 1838 may include autonomous/adaptive/automatic cruise control (ACC), cooperative adaptive cruise control (CACC), forward crash warning (FCW), automatic emergency braking (AEB), lane departure warnings (LDW), lane keep assist (LKA), blind spot warning (BSW), rear cross-traffic warning (RCTW), collision warning systems (CWS), lane centering (LC), and/or other features and functionality.

The ACC systems may use RADAR sensor(s) 1860, LiDAR sensor(s) 1864, and/or a camera(s). The ACC systems may include longitudinal ACC and/or lateral ACC. Longitudinal ACC monitors and controls the distance to the vehicle immediately ahead of the vehicle 1800 and automatically adjust the vehicle speed to maintain a safe distance from vehicles ahead. Lateral ACC performs distance keeping, and advises the vehicle 1800 to change lanes when necessary. Lateral ACC is related to other ADAS applications such as LCA and CWS.

CACC uses information from other vehicles that may be received via the network interface 1824 and/or the wireless antenna(s) 1826 from other vehicles via a wireless link, or indirectly, over a network connection (e.g., over the Internet). Direct links may be provided by a vehicle-to-vehicle (V2V) communication link, while indirect links may be infrastructure-to-vehicle (I2V) communication link. In general, the V2V communication concept provides information about the immediately preceding vehicles (e.g., vehicles immediately ahead of and in the same lane as the vehicle 1800), while the I2V communication concept provides information about traffic further ahead. CACC systems may include either or both I2V and V2V information sources. Given the information of the vehicles ahead of the vehicle 1800, CACC may be more reliable and it has potential to improve traffic flow smoothness and reduce congestion on the road.

FCW systems are designed to alert the driver to a hazard, so that the driver may take corrective action. FCW systems use a front-facing camera and/or RADAR sensor(s) 1860, coupled to a dedicated processor, DSP, FPGA, and/or ASIC, that is electrically coupled to driver feedback, such as a display, speaker, and/or vibrating component. FCW systems may provide a warning, such as in the form of a sound, visual warning, vibration and/or a quick brake pulse.

AEB systems detect an impending forward collision with another vehicle or other object, and may automatically apply the brakes if the driver does not take corrective action within a specified time or distance parameter. AEB systems may use front-facing camera(s) and/or RADAR sensor(s) 1860, coupled to a dedicated processor, DSP, FPGA, and/or ASIC. When the AEB system detects a hazard, it typically first alerts the driver to take corrective action to avoid the collision and, if the driver does not take corrective action, the AEB system may automatically apply the brakes in an effort to prevent, or at least mitigate, the impact of the predicted collision. AEB systems, may include techniques such as dynamic brake support and/or crash imminent braking.

LDW systems provide visual, audible, and/or tactile warnings, such as steering wheel or seat vibrations, to alert the driver when the vehicle 1800 crosses lane markings. A LDW system does not activate when the driver indicates an intentional lane departure, by activating a turn signal. LDW systems may use front-side facing cameras, coupled to a dedicated processor, DSP, FPGA, and/or ASIC, that is electrically coupled to driver feedback, such as a display, speaker, and/or vibrating component.

LKA systems are a variation of LDW systems. LKA systems provide steering input or braking to correct the vehicle 1800 if the vehicle 1800 starts to exit the lane.

BSW systems detects and warn the driver of vehicles in an automobile's blind spot. BSW systems may provide a visual, audible, and/or tactile alert to indicate that merging or changing lanes is unsafe. The system may provide an additional warning when the driver uses a turn signal. BSW systems may use rear-side facing camera(s) and/or RADAR sensor(s) 1860, coupled to a dedicated processor, DSP, FPGA, and/or ASIC, that is electrically coupled to driver feedback, such as a display, speaker, and/or vibrating component.

RCTW systems may provide visual, audible, and/or tactile notification when an object is detected outside the rear-camera range when the vehicle 1800 is backing up. Some RCTW systems include AEB to ensure that the vehicle brakes are applied to avoid a crash. RCTW systems may use one or more rear-facing RADAR sensor(s) 1860, coupled to a dedicated processor, DSP, FPGA, and/or ASIC, that is electrically coupled to driver feedback, such as a display, speaker, and/or vibrating component.

Conventional ADAS systems may be prone to false positive results which may be annoying and distracting to a driver, but typically are not catastrophic, because the ADAS systems alert the driver and allow the driver to decide whether a safety condition truly exists and act accordingly. However, in an autonomous vehicle 1800, the vehicle 1800 itself must, in the case of conflicting results, decide whether to heed the result from a primary computer or a secondary computer (e.g., a first controller 1836 or a second controller 1836). For example, in some embodiments, the ADAS system 1838 may be a backup and/or secondary computer for providing perception information to a backup computer rationality module. The backup computer rationality monitor may run a redundant diverse software on hardware components to detect faults in perception and dynamic driving tasks. Outputs from the ADAS system 1838 may be provided to a supervisory MCU. If outputs from the primary computer and the secondary computer conflict, the supervisory MCU must determine how to reconcile the conflict to ensure safe operation.

In some examples, the primary computer may be configured to provide the supervisory MCU with a confidence score, indicating the primary computer's confidence in the chosen result. If the confidence score exceeds a threshold, the supervisory MCU may follow the primary computer's direction, regardless of whether the secondary computer provides a conflicting or inconsistent result. Where the confidence score does not meet the threshold, and where the primary and secondary computer indicate different results (e.g., the conflict), the supervisory MCU may arbitrate between the computers to determine the appropriate outcome.

The supervisory MCU may be configured to run a neural network(s) that is trained and configured to determine, based on outputs from the primary computer and the secondary computer, conditions under which the secondary computer provides false alarms. Thus, the neural network(s) in the supervisory MCU may learn when the secondary computer's output may be trusted, and when it cannot. For example, when the secondary computer is a RADAR-based FCW system, a neural network(s) in the supervisory MCU may learn when the FCW system is identifying metallic objects that are not, in fact, hazards, such as a drainage grate or manhole cover that triggers an alarm. Similarly, when the secondary computer is a camera-based LDW system, a neural network in the supervisory MCU may learn to override the LDW when bicyclists or pedestrians are present and a lane departure is, in fact, the safest maneuver. In embodiments that include a neural network(s) running on the supervisory MCU, the supervisory MCU may include at least one of a DLA or GPU suitable for running the neural network(s) with associated memory. In preferred embodiments, the supervisory MCU may comprise and/or be included as a component of the SoC(s) 1804.

In other examples, ADAS system 1838 may include a secondary computer that performs ADAS functionality using traditional rules of computer vision. As such, the secondary computer may use classic computer vision rules (if-then), and the presence of a neural network(s) in the supervisory MCU may improve reliability, safety and performance. For example, the diverse implementation and intentional non-identity makes the overall system more fault-tolerant, especially to faults caused by software (or software-hardware interface) functionality. For example, if there is a software bug or error in the software running on the primary computer, and the non-identical software code running on the secondary computer provides the same overall result, the supervisory MCU may have greater confidence that the overall result is correct, and the bug in software or hardware on primary computer is not causing material error.

In some examples, the output of the ADAS system 1838 may be fed into the primary computer's perception block and/or the primary computer's dynamic driving task block. For example, if the ADAS system 1838 indicates a forward crash warning due to an object immediately ahead, the perception block may use this information when identifying objects. In other examples, the secondary computer may have its own neural network which is trained and thus reduces the risk of false positives, as described herein.

The vehicle 1800 may further include the infotainment SoC 1830 (e.g., an in-vehicle infotainment system (IVI)). Although illustrated and described as a SoC, the infotainment system may not be a SoC, and may include two or more discrete components. The infotainment SoC 1830 may include a combination of hardware and software that may be used to provide audio (e.g., music, a personal digital assistant, navigational instructions, news, radio, etc.), video (e.g., TV, movies, streaming, etc.), phone (e.g., hands-free calling), network connectivity (e.g., LTE, Wi-Fi, etc.), and/or information services (e.g., navigation systems, rear-parking assistance, a radio data system, vehicle related information such as fuel level, total distance covered, brake fuel level, oil level, door open/close, air filter information, etc.) to the vehicle 1800. For example, the infotainment SoC 1830 may radios, disk players, navigation systems, video players, USB and Bluetooth connectivity, carputers, in-car entertainment, Wi-Fi, steering wheel audio controls, hands free voice control, a heads-up display (HUD), an HMI display 1834, a telematics device, a control panel (e.g., for controlling and/or interacting with various components, features, and/or systems), and/or other components. The infotainment SoC 1830 may further be used to provide information (e.g., visual and/or audible) to a user(s) of the vehicle, such as information from the ADAS system 1838, autonomous driving information such as planned vehicle maneuvers, trajectories, surrounding environment information (e.g., intersection information, vehicle information, road information, etc.), and/or other information.

The infotainment SoC 1830 may include GPU functionality. The infotainment SoC 1830 may communicate over the bus 1802 (e.g., CAN bus, Ethernet, etc.) with other devices, systems, and/or components of the vehicle 1800. In some examples, the infotainment SoC 1830 may be coupled to a supervisory MCU such that the GPU of the infotainment system may perform some self-driving functions in the event that the primary controller(s) 1836 (e.g., the primary and/or backup computers of the vehicle 1800) fail. In such an example, the infotainment SoC 1830 may put the vehicle 1800 into a chauffeur to safe stop mode, as described herein.

The vehicle 1800 may further include an instrument cluster 1832 (e.g., a digital dash, an electronic instrument cluster, a digital instrument panel, etc.). The instrument cluster 1832 may include a controller and/or supercomputer (e.g., a discrete controller or supercomputer). The instrument cluster 1832 may include a set of instrumentation such as a speedometer, fuel level, oil pressure, tachometer, odometer, turn indicators, gearshift position indicator, seat belt warning light(s), parking-brake warning light(s), engine-malfunction light(s), airbag (SRS) system information, lighting controls, safety system controls, navigation information, etc. In some examples, information may be displayed and/or shared among the infotainment SoC 1830 and the instrument cluster 1832. As such, the instrument cluster 1832 may be included as part of the infotainment SoC 1830, or vice versa.

FIG. 18D is a system diagram for communication between cloud-based server(s) and the example autonomous vehicle 1800 of FIG. 18A, in accordance with some embodiments of the present disclosure. The system 1876 may include server(s) 1878, network(s) 1890, and vehicles, including the vehicle 1800. The server(s) 1878 may include a plurality of GPUs 1884(A)-1884(H) (collectively referred to herein as GPUs 1884), PCIe switches 1882(A)-1882(D) (collectively referred to herein as PCIe switches 1882), and/or CPUs 1880(A)-1880(B) (collectively referred to herein as CPUs 1880). The GPUs 1884, the CPUs 1880, and the PCIe switches may be interconnected with high-speed interconnects such as, for example and without limitation, NVLink interfaces 1888 developed by NVIDIA and/or PCIe connections 1886. In some examples, the GPUs 1884 are connected via NVLink and/or NVSwitch SoC and the GPUs 1884 and the PCIe switches 1882 are connected via PCIe interconnects. Although eight GPUs 1884, two CPUs 1880, and two PCIe switches are illustrated, this is not intended to be limiting. Depending on the embodiment, each of the server(s) 1878 may include any number of GPUs 1884, CPUs 1880, and/or PCIe switches. For example, the server(s) 1878 may each include eight, sixteen, thirty-two, and/or more GPUs 1884.

The server(s) 1878 may receive, over the network(s) 1890 and from the vehicles, image data representative of images showing unexpected or changed road conditions, such as recently commenced road-work. The server(s) 1878 may transmit, over the network(s) 1890 and to the vehicles, neural networks 1892, updated neural networks 1892, and/or map information 1894, including information regarding traffic and road conditions. The updates to the map information 1894 may include updates for the HD map 1822, such as information regarding construction sites, potholes, detours, flooding, and/or other obstructions. In some examples, the neural networks 1892, the updated neural networks 1892, and/or the map information 1894 may have resulted from new training and/or experiences represented in data received from any number of vehicles in the environment, and/or based on training performed at a datacenter (e.g., using the server(s) 1878 and/or other servers).

The server(s) 1878 may be used to train machine learning models (e.g., neural networks) based on training data. The training data may be generated using the vehicles, and/or may be generated in a simulation (e.g., using a game engine). In some examples, the training data is tagged (e.g., where the neural network benefits from supervised learning) and/or undergoes other pre-processing, while in other examples the training data is not tagged and/or pre-processed (e.g., where the neural network does not require supervised learning). Training may be executed according to any one or more classes of machine learning techniques, including, without limitation, classes such as: supervised training, semi-supervised training, unsupervised training, self-learning, reinforcement learning, federated learning, transfer learning, feature learning (including principal component and cluster analyses), multi-linear subspace learning, manifold learning, representation learning (including spare dictionary learning), rule-based machine learning, anomaly detection, and any variants or combinations therefor. Once the machine learning models are trained, the machine learning models may be used by the vehicles (e.g., transmitted to the vehicles over the network(s) 1890, and/or the machine learning models may be used by the server(s) 1878 to remotely monitor the vehicles.

In some examples, the server(s) 1878 may receive data from the vehicles and apply the data to up-to-date real-time neural networks for real-time intelligent inferencing. The server(s) 1878 may include deep-learning supercomputers and/or dedicated AI computers powered by GPU(s) 1884, such as a DGX and DGX Station machines developed by NVIDIA. However, in some examples, the server(s) 1878 may include deep learning infrastructure that use only CPU-powered datacenters.

The deep-learning infrastructure of the server(s) 1878 may be capable of fast, real-time inferencing, and may use that capability to evaluate and verify the health of the processors, software, and/or associated hardware in the vehicle 1800. For example, the deep-learning infrastructure may receive periodic updates from the vehicle 1800, such as a sequence of images and/or objects that the vehicle 1800 has located in that sequence of images (e.g., via computer vision and/or other machine learning object classification techniques). The deep-learning infrastructure may run its own neural network to identify the objects and compare them with the objects identified by the vehicle 1800 and, if the results do not match and the infrastructure concludes that the AI in the vehicle 1800 is malfunctioning, the server(s) 1878 may transmit a signal to the vehicle 1800 instructing a fail-safe computer of the vehicle 1800 to assume control, notify the passengers, and complete a safe parking maneuver.

For inferencing, the server(s) 1878 may include the GPU(s) 1884 and one or more programmable inference accelerators (e.g., NVIDIA's Tensor®). The combination of GPU-powered servers and inference acceleration may make real-time responsiveness possible. In other examples, such as where performance is less critical, servers powered by CPUs, FPGAs, and other processors may be used for inferencing.

### Inference And Training Logic

One or more embodiments may be implemented using inference and/or training logic to perform inferencing and/or training operations. Details regarding inference and/or training logic are provided below.

In at least one embodiment, inference and/or training logic may include, without limitation, code and/or data storage to store forward and/or output weight and/or input/output data, and/or other parameters to configure. neurons or layers of a neural network trained and/or used for inferencing in aspects of one or more embodiments. In at least one embodiment, training logic may include, or be coupled to code and/or data storage to store graph code or other software to control timing and/or order, in which weight and/or other parameter information is to be loaded to configure., logic, including integer and/or floating point units (collectively, arithmetic logic units (ALUs). In at least one embodiment, code, such as graph code, loads weight or other parameter information into processor ALUs based on an architecture of a neural network to which the code corresponds. In at least one embodiment, code and/or data storage stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage may be included with other on-chip or off-chip data storage, including a processor's L1, L2, or L3 cache or system memory.

In at least one embodiment, any portion of code and/or data storage may be internal or external to one or more processors or other hardware logic devices or circuits. In at least one embodiment, code and/or data storage may be cache memory, dynamic randomly addressable memory (“DRAM”), static randomly addressable memory (“SRAM”), non-volatile memory (e.g., Flash memory), or other storage. In at least one embodiment, choice of whether code and/or data storage is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic may include, without limitation, a code and/or data storage to store backward and/or output weight and/or input/output data corresponding to neurons or layers of a neural network trained and/or used for inferencing in aspects of one or more embodiments. In at least one embodiment, code and/or data storage stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic may include, or be coupled to code and/or data storage to store graph code or other software to control timing and/or order, in which weight and/or other parameter information is to be loaded to configure., logic, including integer and/or floating point units (collectively, arithmetic logic units (ALUs). In at least one embodiment, code, such as graph code, loads weight or other parameter information into processor ALUs based on an architecture of a neural network to which the code corresponds. In at least one embodiment, any portion of code and/or data storage may be included with other on-chip or off-chip data storage, including a processor's L1, L2, or L3 cache or system memory. In at least one embodiment, any portion of code and/or data storage may be internal or external to on one or more processors or other hardware logic devices or circuits. In at least one embodiment, code and/or data storage may be cache memory, DRAM, SRAM, non-volatile memory (e.g., Flash memory), or other storage. In at least one embodiment, choice of whether code and/or data storage is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage and code and/or data storage may be separate storage structures. In at least one embodiment, code and/or data storage and code and/or data storage may be same storage structure. In at least one embodiment, code and/or data storage and code and/or data storage may be partially same storage structure and partially separate storage structures. In at least one embodiment, any portion of code and/or data storage and code and/or data storage may be included with other on-chip or off-chip data storage, including a processor's L1, L2, or L3 cache or system memory.

In at least one embodiment, inference and/or training logic may include, without limitation, one or more arithmetic logic unit(s) (“ALU(s)”), including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage that are functions of input/output and/or weight parameter data stored in code and/or data storage and/or code and/or data storage. In at least one embodiment, activations stored in activation storage are generated according to linear algebraic and or matrix-based mathematics performed by ALU(s) in response to performing instructions or other code, wherein weight values stored in code and/or data storage and/or code and/or data storage are used as operands along with other values, such as bias values, gradient information, momentum values, or other parameters or hyperparameters, any or all of which may be stored in code and/or data storage or code and/or data storage or another storage on or off-chip.

In at least one embodiment, ALU(s) are included within one or more processors or other hardware logic devices or circuits, whereas in another embodiment, ALU(s) may be external to a processor or other hardware logic device or circuit that uses them (e.g., a co-processor). In at least one embodiment, ALU(s) may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage, code and/or data storage, and activation storage may be on same processor or other hardware logic device or circuit, whereas in another embodiment, they may be in different processors or other hardware logic devices or circuits, or some combination of same and different processors or other hardware logic devices or circuits. In at least one embodiment, any portion of activation storage may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage may be cache memory, DRAM, SRAM, non-volatile memory (e.g., Flash memory), or other storage. In at least one embodiment, activation storage may be completely or partially within or external to one or more processors or other logical circuits. In at least one embodiment, choice of whether activation storage is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors. In at least one embodiment, inference and/or training logic may be used in conjunction with an application-specific integrated circuit (“ASIC”), such as Tensorflow® Processing Unit from Google, an inference processing unit (IPU) from Graphcore™, or a Nervana® (e.g., “Lake Crest”) processor from Intel Corp. In at least one embodiment, inference and/or training logic may be used in conjunction with central processing unit (“CPU”) hardware, graphics processing unit (“GPU”) hardware or other hardware, such as field programmable gate arrays (“FPGAs”).

In at least one embodiment, inference and/or training logic may include, without limitation, hardware logic in which computational resources are dedicated or otherwise exclusively used in conjunction with weight values or other information corresponding to one or more layers of neurons within a neural network. In at least one embodiment, inference and/or training logic may be used in conjunction with an application-specific integrated circuit (ASIC), such as Tensorflow® Processing Unit from Google, an inference processing unit (IPU) from Graphcore™, or a Nervana® (e.g., “Lake Crest”) processor from Intel Corp. In at least one embodiment, inference and/or training logic may be used in conjunction with central processing unit (CPU) hardware, graphics processing unit (GPU) hardware or other hardware, such as field programmable gate arrays (FPGAs). In at least one embodiment, inference and/or training logic includes, without limitation, code and/or data storage and code and/or data storage, which may be used to store code (e.g., graph code), weight values and/or other information, including bias values, gradient information, momentum values, and/or other parameter or hyperparameter information. In at least one embodiment, each of code and/or data storage and code and/or data storage is associated with a dedicated computational resource, such as computational hardware and computational hardware. In at least one embodiment, each of computational hardware and computational hardware comprises one or more ALUs that perform mathematical functions, such as linear algebraic functions, only on information stored in code and/or data storage and code and/or data storage, respectively, result of which is stored in activation storage.

In at least one embodiment, each of code and/or data storage and corresponding computational hardware correspond to different layers of a neural network, such that resulting activation from one storage/computational pair of code and/or data storage and computational hardware is provided as an input to storage/computational pair of code and/or data storage and computational hardware, in order to mirror conceptual organization of a neural network. In at least one embodiment, each of storage/computational pairs may correspond to more than one neural network layer. In at least one embodiment, additional storage/computation pairs (not shown) subsequent to or in parallel with storage computation pairs may be included in inference and/or training logic.

### Example Computing Device

FIG. 19 is a block diagram of an example computing device(s) 1900 suitable for use in implementing some embodiments of the present disclosure. Computing device 1900 may include an interconnect system 1902 that directly or indirectly couples the following devices: memory 1904, one or more central processing units (CPUs) 1906, one or more graphics processing units (GPUs) 1908, a communication interface 1910, input/output (I/O) ports 1912, input/output components 1914, a power supply 1916, one or more presentation components 1918 (e.g., display(s)), and one or more logic units 1920. In at least one embodiment, the computing device(s) 1900 may comprise one or more virtual machines (VMs), and/or any of the components thereof may comprise virtual components (e.g., virtual hardware components). For non-limiting examples, one or more of the GPUs 1908 may comprise one or more vGPUs, one or more of the CPUs 1906 may comprise one or more vCPUs, and/or one or more of the logic units 1920 may comprise one or more virtual logic units. As such, a computing device(s) 1900 may include discrete components (e.g., a full GPU dedicated to the computing device 1900), virtual components (e.g., a portion of a GPU dedicated to the computing device 1900), or a combination thereof.

Although the various blocks of FIG. 19 are shown as connected via the interconnect system 1902 with lines, this is not intended to be limiting and is for clarity only. For example, in some embodiments, a presentation component 1918, such as a display device, may be considered an I/O component 1914 (e.g., if the display is a touch screen). As another example, the CPUs 1906 and/or GPUs 1908 may include memory (e.g., the memory 1904 may be representative of a storage device in addition to the memory of the GPUs 1908, the CPUs 1906, and/or other components). As such, the computing device of FIG. 19 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” and/or other device or system types, as all are contemplated within the scope of the computing device of FIG. 19.

The interconnect system 1902 may represent one or more links or busses, such as an address bus, a data bus, a control bus, or a combination thereof. The interconnect system 1902 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 1906 may be directly connected to the memory 1904. Further, the CPU 1906 may be directly connected to the GPU 1908. Where there is direct, or point-to-point connection between components, the interconnect system 1902 may include a PCIe link to carry out the connection. In these examples, a PCI bus need not be included in the computing device 1900.

The memory 1904 may include any of a variety of computer-readable media. The computer-readable media may be any available media that may be accessed by the computing device 1900. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 1904 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which may be used to store the desired information and which may be accessed by computing device 1900. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 1906 may be configured to execute at least some of the computer-readable instructions to control one or more components of the computing device 1900 to perform one or more of the methods and/or processes described herein. The CPU(s) 1906 may each include one or more cores (e.g., one, two, four, eight, twenty-eight, seventy-two, etc.) that are capable of handling a multitude of software threads simultaneously. The CPU(s) 1906 may include any type of processor, and may include different types of processors depending on the type of computing device 1900 implemented (e.g., processors with fewer cores for mobile devices and processors with more cores for servers). For example, depending on the type of computing device 1900, the processor may be an Advanced RISC Machines (ARM) processor implemented using Reduced Instruction Set Computing (RISC) or an x86 processor implemented using Complex Instruction Set Computing (CISC). The computing device 1900 may include one or more CPUs 1906 in addition to one or more microprocessors or supplementary co-processors, such as math co-processors.

In addition to or alternatively from the CPU(s) 1906, the GPU(s) 1908 may be configured to execute at least some of the computer-readable instructions to control one or more components of the computing device 1900 to perform one or more of the methods and/or processes described herein. One or more of the GPU(s) 1908 may be an integrated GPU (e.g., with one or more of the CPU(s) 1906 and/or one or more of the GPU(s) 1908 may be a discrete GPU. In embodiments, one or more of the GPU(s) 1908 may be a coprocessor of one or more of the CPU(s) 1906. The GPU(s) 1908 may be used by the computing device 1900 to render graphics (e.g., 3D graphics) or perform general purpose computations. For example, the GPU(s) 1908 may be used for General-Purpose computing on GPUs (GPGPU). The GPU(s) 1908 may include hundreds or thousands of cores that are capable of handling hundreds or thousands of software threads simultaneously. The GPU(s) 1908 may generate pixel data for output images in response to rendering commands (e.g., rendering commands from the CPU(s) 1906 received via a host interface). The GPU(s) 1908 may include graphics memory, such as display memory, for storing pixel data or any other suitable data, such as GPGPU data. The display memory may be included as part of the memory 1904. The GPU(s) 1908 may include two or more GPUs operating in parallel (e.g., via a link). The link may directly connect the GPUs (e.g., using NVLINK) or may connect the GPUs through a switch (e.g., using NVSwitch). When combined together, each GPU 1908 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 1906 and/or the GPU(s) 1908, the logic unit(s) 1920 may be configured to execute at least some of the computer-readable instructions to control one or more components of the computing device 1900 to perform one or more of the methods and/or processes described herein. In embodiments, the CPU(s) 1906, the GPU(s) 1908, and/or the logic unit(s) 1920 may discretely or jointly perform any combination of the methods, processes and/or portions thereof. One or more of the logic units 1920 may be part of and/or integrated in one or more of the CPU(s) 1906 and/or the GPU(s) 1908 and/or one or more of the logic units 1920 may be discrete components or otherwise external to the CPU(s) 1906 and/or the GPU(s) 1908. In embodiments, one or more of the logic units 1920 may be a coprocessor of one or more of the CPU(s) 1906 and/or one or more of the GPU(s) 1908.

Examples of the logic unit(s) 1920 include one or more processing cores and/or components thereof, such as Data Processing Units (DPUs), Tensor Cores (TCs), Tensor Processing Units(TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), input/output (I/O) elements, peripheral component interconnect (PCI) or peripheral component interconnect express (PCIe) elements, and/or the like.

The communication interface 1910 may include one or more receivers, transmitters, and/or transceivers that allow the computing device 1900 to communicate with other computing devices via an electronic communication network, included wired and/or wireless communications. The communication interface 1910 may include components and functionality to allow communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet. In one or more embodiments, logic unit(s) 1920 and/or communication interface 1910 may include one or more data processing units (DPUs) to transmit data received over a network and/or through interconnect system 1902 directly to (e.g., a memory of) one or more GPU(s) 1908.

The I/O ports 1912 may allow the computing device 1900 to be logically coupled to other devices including the I/O components 1914, the presentation component(s) 1918, and/or other components, some of which may be built in to (e.g., integrated in) the computing device 1900. Illustrative I/O components 1914 include a microphone, mouse, keyboard, joystick, game pad, game controller, satellite dish, scanner, printer, wireless device, etc. The I/O components 1914 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing device 1900. The computing device 1900 may be include depth cameras, such as stereoscopic camera systems, infrared camera systems, RGB camera systems, touchscreen technology, and combinations of these, for gesture detection and recognition. Additionally, the computing device 1900 may include accelerometers or gyroscopes (e.g., as part of an inertia measurement unit (IMU)) that allow detection of motion. In some examples, the output of the accelerometers or gyroscopes may be used by the computing device 1900 to render immersive augmented reality or virtual reality.

The power supply 1916 may include a hard-wired power supply, a battery power supply, or a combination thereof. The power supply 1916 may provide power to the computing device 1900 to allow the components of the computing device 1900 to operate.

The presentation component(s) 1918 may include a display (e.g., a monitor, a touch screen, a television screen, a heads-up-display (HUD), other display types, or a combination thereof), speakers, and/or other presentation components. The presentation component(s) 1918 may receive data from other components (e.g., the GPU(s) 1908, the CPU(s) 1906, DPUs, etc.), and output the data (e.g., as an image, video, sound, etc.).

### Example Data Center

FIG. 20 illustrates an example data center 2000 that may be used in at least one embodiments of the present disclosure. The data center 2000 may include a data center infrastructure layer 2010, a framework layer 2020, a software layer 2030, and/or an application layer 2040.

As shown in FIG. 20, the data center infrastructure layer 2010 may include a resource orchestrator 2012, grouped computing resources 2014, and node computing resources (“node C.R.s”) 2016(1)-2016(N), where “N” represents any whole, positive integer. In at least one embodiment, node C.R.s 2016(1)-2016(N) may include, but are not limited to, any number of central processing units (CPUs) or other processors (including DPUs, accelerators, field programmable gate arrays (FPGAs), graphics processors or graphics processing units (GPUs), etc.), memory devices (e.g., dynamic read-only memory), storage devices (e.g., solid state or disk drives), network input/output (NW I/O) devices, network switches, virtual machines (VMs), power modules, and/or cooling modules, etc. In some embodiments, one or more node C.R.s from among node C.R.s 2016(1)-2016(N) may correspond to a server having one or more of the above-mentioned computing resources. In addition, in some embodiments, the node C.R.s 2016(1)-20161(N) may include one or more virtual components, such as vGPUs, vCPUs, and/or the like, and/or one or more of the node C.R.s 2016(1)-2016(N) may correspond to a virtual machine (VM).

In at least one embodiment, grouped computing resources 2014 may include separate groupings of node C.R.s 2016 housed within one or more racks (not shown), or many racks housed in data centers at various geographical locations (also not shown). Separate groupings of node C.R.s 2016 within grouped computing resources 2014 may include grouped compute, network, memory or storage resources that may be configured or allocated to support one or more workloads. In at least one embodiment, several node C.R.s 2016 including CPUs, GPUs, DPUs, and/or other processors may be grouped within one or more racks to provide compute resources to support one or more workloads. The one or more racks may also include any number of power modules, cooling modules, and/or network switches, in any combination.

The resource orchestrator 2012 may configure or otherwise control one or more node C.R.s 2016(1)-2016(N) and/or grouped computing resources 2014. In at least one embodiment, resource orchestrator 2012 may include a software design infrastructure (SDI) management entity for the data center 2000. The resource orchestrator 2012 may include hardware, software, or some combination thereof.

In at least one embodiment, as shown in FIG. 20, framework layer 2020 may include a job scheduler 2033, a configuration manager 2034, a resource manager 2036, and/or a distributed file system 2038. The framework layer 2020 may include a framework to support software 2032 of software layer 2030 and/or one or more application(s) 2042 of application layer 2040. The software 2032 or application(s) 2042 may respectively include web-based service software or applications, such as those provided by Amazon Web Services, Google Cloud and Microsoft Azure. The framework layer 2020 may be, but is not limited to, a type of free and open-source software web application framework such as Apache Spark™ (hereinafter “Spark”) that may use distributed file system 2038 for large-scale data processing (e.g., “big data”). In at least one embodiment, job scheduler 2033 may include a Spark driver to facilitate scheduling of workloads supported by various layers of data center 2000. The configuration manager 2034 may be capable of configuring different layers such as software layer 2030 and framework layer 2020 including Spark and distributed file system 2038 for supporting large-scale data processing. The resource manager 2036 may be capable of managing clustered or grouped computing resources mapped to or allocated for support of distributed file system 2038 and job scheduler 2033. In at least one embodiment, clustered or grouped computing resources may include grouped computing resource 2014 at data center infrastructure layer 2010. The resource manager 2036 may coordinate with resource orchestrator 2012 to manage these mapped or allocated computing resources.

In at least one embodiment, software 2032 included in software layer 2030 may include software used by at least portions of node C.R.s 2016(1)-2016(N), grouped computing resources 2014, and/or distributed file system 2038 of framework layer 2020. One or more types of software may include, but are not limited to, Internet web page search software, e-mail virus scan software, database software, and streaming video content software.

In at least one embodiment, application(s) 2042 included in application layer 2040 may include one or more types of applications used by at least portions of node C.R.s 2016(1)-2016(N), grouped computing resources 2014, and/or distributed file system 2038 of framework layer 2020. One or more types of applications may include, but are not limited to, any number of a genomics application, a cognitive compute, and a machine learning application, including training or inferencing software, machine learning framework software (e.g., PyTorch, TensorFlow, Caffe, etc.), and/or other machine learning applications used in conjunction with one or more embodiments.

In at least one embodiment, any of configuration manager 2034, resource manager 2036, and resource orchestrator 2012 may implement any number and type of self-modifying actions based on any amount and type of data acquired in any technically feasible fashion. Self-modifying actions may relieve a data center operator of data center 2000 from making possibly bad configuration decisions and possibly avoiding underutilized and/or poor performing portions of a data center.

The data center 2000 may include tools, services, software or other resources to train one or more machine learning models or predict or infer information using one or more machine learning models according to one or more embodiments described herein. For example, a machine learning model(s) may be trained by calculating weight parameters according to a neural network architecture using software and/or computing resources described above with respect to the data center 2000. In at least one embodiment, trained or deployed machine learning models corresponding to one or more neural networks may be used to infer or predict information using resources described above with respect to the data center 2000 by using weight parameters calculated through one or more training techniques, such as but not limited to those described herein.

In at least one embodiment, the data center 2000 may use CPUs, application-specific integrated circuits (ASICs), GPUs, FPGAs, and/or other hardware (or virtual compute resources corresponding thereto) to perform training and/or inferencing using above-described resources. Moreover, one or more software and/or hardware resources described above may be configured as a service to allow users to train or performing inferencing of information, such as image recognition, speech recognition, or other artificial intelligence services.

### Example Network Environments

Network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing device(s) 1900 of FIG. 19—e.g., each device may include similar components, features, and/or functionality of the computing device(s) 1900. In addition, where backend devices (e.g., servers, NAS, etc.) are implemented, the backend devices may be included as part of a data center 2000, an example of which is described in more detail herein with respect to FIG. 20.

Components of a network environment may communicate with each other via a network(s), which may be wired, wireless, or both. The network may include multiple networks, or a network of networks. By way of example, the network may include one or more Wide Area Networks (WANs), one or more Local Area Networks (LANs), one or more public networks such as the Internet and/or a public switched telephone network (PSTN), and/or one or more private networks. Where the network includes a wireless telecommunications network, components such as a base station, a communications tower, or even access points (as well as other components) may provide wireless connectivity.

Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may include a framework layer, a job scheduler, a resource manager, and a distributed file system implemented on one or more of servers, which may include one or more core network servers and/or edge servers. A framework layer may include a framework to support software of a software layer and/or one or more application(s) of an application layer. The software or application(s) may respectively include web-based service software or applications. In embodiments, one or more of the client devices may use the web-based service software or applications (e.g., by accessing the service software and/or applications via one or more application programming interfaces (APIs)). The framework layer may be, but is not limited to, a type of free and open-source software web application framework such as that may use a distributed file system for large-scale data processing (e.g., “big data”).

A cloud-based network environment may provide cloud computing and/or cloud storage that carries out any combination of computing and/or data storage functions described herein (or one or more portions thereof). Any of these various functions may be distributed over multiple locations from central or core servers (e.g., of one or more data centers that may be distributed across a state, a region, a country, the globe, etc.). If a connection to a user (e.g., a client device) is relatively close to an edge server(s), a core server(s) may designate at least a portion of the functionality to the edge server(s). A cloud-based network environment may be private (e.g., limited to a single organization), may be public (e.g., available to many organizations), and/or a combination thereof (e.g., a hybrid cloud environment).

The client device(s) may include at least some of the components, features, and functionality of the example computing device(s) 1900 described herein with respect to FIG. 19. By way of example and not limitation, a client device may be embodied as a Personal Computer (PC), a laptop computer, a mobile device, a smartphone, a tablet computer, a smart watch, a wearable computer, a Personal Digital Assistant (PDA), an MP3 player, a virtual reality headset, a Global Positioning System (GPS) or device, a video player, a video camera, a surveillance device or system, a vehicle, a boat, a flying vessel, a virtual machine, a drone, a robot, a handheld communications device, a hospital device, a gaming device or system, an entertainment system, a vehicle computer system, an embedded system controller, a remote control, an appliance, a consumer electronic device, a workstation, an edge device, any combination of these delineated devices, or any other suitable device.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular abstract data types. The disclosure may be practiced in a variety of system configurations, including hand-held devices, consumer electronics, general-purpose computers, more specialty computing devices, etc. The disclosure may also be practiced in distributed computing environments where tasks are performed by remote-processing devices that are linked through a communications network.

Other variations are within the spirit of present disclosure. Thus, while disclosed techniques are susceptible to various modifications and alternative constructions, certain illustrated embodiments thereof are shown in drawings and have been described above in detail. It should be understood, however, that there is no intention to limit disclosure to specific form or forms disclosed, but on contrary, intention is to cover all modifications, alternative constructions, and equivalents falling within spirit and scope of disclosure, as defined in the appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. Term “connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. Use of term “set” (e.g., “a set of items”) or “subset,” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in an illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B, and at least one of C each to be present. In addition, unless otherwise noted or contradicted by context, term “plurality” indicates a state of being plural (e.g., “a plurality of items” indicates multiple items). A plurality is at least two items, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. A set of non-transitory computer-readable storage media, in at least one embodiment, comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors-for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

Accordingly, in at least one embodiment, computer systems are configured to implement one or more services that singly or collectively perform operations of processes described herein and such computer systems are configured with applicable hardware and/or software that allow performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. Terms “system” and “method” are used herein interchangeably as far as system may embody one or more methods and methods may be considered a system.

In the present document, references may be made to obtaining, acquiring, receiving, or inputting analog or digital data into a subsystem, computer system, or computer-implemented machine. Obtaining, acquiring, receiving, or inputting analog and digital data can be accomplished in a variety of ways such as by receiving data as a parameter of a function call or a call to an application programming interface. In some implementations, process of obtaining, acquiring, receiving, or inputting analog or digital data can be accomplished by transferring data via a serial or parallel interface. In another implementation, process of obtaining, acquiring, receiving, or inputting analog or digital data can be accomplished by transferring data via a computer network from providing entity to acquiring entity. References may also be made to providing, outputting, transmitting, sending, or presenting analog or digital data. In various examples, process of providing, outputting, transmitting, sending, or presenting analog or digital data can be accomplished by transferring data as an input or output parameter of a function call, a parameter of an application programming interface or interprocess communication mechanism.

Although the discussion above sets forth example implementations of described techniques, other architectures may be used to implement described functionality, and are intended to be within scope of this disclosure. Furthermore, although specific distributions of responsibilities are defined above for purposes of discussion, various functions and responsibilities might be distributed and divided in different ways, depending on circumstances.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims. The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

### Example Literal Support

The disclosure of this application also includes the following numbered clauses:
Clause 1. One or more processors comprising processing circuitry to receive two or more types of detection results representative of presence of one or more detected occupants in one or more occupant slots in an interior space.

Clause 2. The one or more processors of clause 1, wherein the processing circuitry is further to generate a representation of whether a child is present in at least one occupant slot of the one or more occupant slots based at least on executing slot-dependent logic associated with the one or more occupant slots to combine the two or more types of detection results representative of predicted presence.

Clause 3. The one or more processors of clause 1 or 2, wherein the processing circuitry is further to execute one or more operations based at least on the representation of whether the child is present.

Clause 4. The one or more processors of clause 1, 2 or 3, wherein the slot-dependent logic includes size-based age estimation of occupants in at least a first occupant slot of the one or more occupant slots, and omits size-based age estimation in at least a second occupant slot of the one or more occupant slots.

Clause 5. The one or more processors of clause 1, 2 or 3, wherein the slot-dependent logic comprises determining whether RADAR liveness detection classifies at least a first occupant slot of the one or more occupant slots as occupied based at least on determining that RADAR blockage detection classifies a view of the first occupant slot as not blocked.

Clause 6. The one or more processors of clause 1, 2 or 3, wherein the slot-dependent logic comprises 1) a first determination of whether camera presence or occupancy detection classifies at least a first occupant slot of the one or more occupant slots as occupied, 2) a second determination of whether RADAR liveness detection classifies at least the first occupant slot as occupied, and 3) a third determination of whether at least one of the first or second determinations classified at least the first occupant slot as occupied.

Clause 7. The one or more processors of clause 1, 2 or 3, wherein the slot-dependent logic comprises generating, based at least on determining that camera presence or occupancy detection classifies at least a first occupant slot of the one or more occupant slots as occupied, a representation of whether the first occupant slot is occupied by a child or an adult based at least on combining prediction confidences generated by child seat detection, face-based age estimation, and size-based age estimation.

Clause 8. The one or more processors of clause 1, 2 or 3, wherein the slot-dependent logic comprises determining whether RADAR pet detection classifies at least a first occupant slot of the one or more occupant slots as occupied by a human-like signature based at least on determining that RADAR liveness detection classifies the first occupant slot as occupied.

Clause 9. The one or more processors of clause 1, 2 or 3, wherein the slot-dependent logic comprises a first branch triggered by RADAR occupant classification of a child, a second branch triggered by RADAR occupant classification of an adult, and third branch triggered by RADAR occupant classification as an unknown occupant.

Clause 10. The one or more processors of clause 1, 2 or 3, wherein the slot-dependent logic comprises, based at least on RADAR occupant classification of a child in at least a first occupant slot of the one or more occupant slots, determining whether camera presence or occupancy detection classifies at least the first occupant slot as occupied.

Clause 11. The one or more processors of clause 1, 2 or 3, wherein the slot-dependent logic comprises, based at least on RADAR occupant classification of an adult in at least a first occupant slot of the one or more occupant slots, determining whether child seat detection classifies the first occupant slot as occupied by a child seat.

Clause 12. The one or more processors of clause 1, 2 or 3, wherein the slot-dependent logic comprises, based at least on RADAR occupant classification of an unknown occupant in at least a first occupant slot of the one or more occupant slots, determining whether child seat detection classifies the first occupant slot as occupied by a child seat.

Clause 13. The one or more processors of clause 1, 2 or 3, wherein the slot-dependent logic comprises executing RADAR age estimation based at least on determining that RADAR pet detection classifies at least a first occupant slot of the one or more occupant slots as occupied by a human-like signature.

Clause 14. The one or more processors of clause 1, 2 or 3, wherein the processing circuitry is further to determine, based at least on determining that the child is present in the at least one occupant slot, whether an adult is present in at least one other occupant slot of the one or more occupant slots.

Clause 15. The one or more processors of clause 1, 2 or 3, wherein the one or more processors are comprised in at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporates one or more machine learning models deployed in a service or microservice along with an OS-level virtualization package (e.g., a container).

Clause 16. A method comprising generating a representation of whether a child is present in at least one occupant slot of one or more occupant slots based at least on executing slot-dependent logic to combine two or more types of detection results representative of occupant presence.

Clause 17. The method of clause 16, further comprising executing one or more operations based at least on the representation of whether the child is present in the at least one occupant slot..

Clause 18. The method of clause 16 or 17, wherein the slot-dependent logic includes size-based age estimation of occupants in at least a first occupant slot of the one or more occupant slots, and omits size-based age estimation in at least a second occupant slot of the one or more occupant slots.

Clause 19. The method of clause 16 or 17, wherein the slot-dependent logic comprises determining whether RADAR liveness detection classifies at least a first occupant slot of the one or more occupant slots as occupied based at least on determining that RADAR blockage detection classifies a view of the first occupant slot as not blocked.

Clause 20. The method of clause 16 or 17, wherein the method is performed by at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporates one or more machine learning models deployed in a service or microservice along with an OS-level virtualization package (e.g., a container).

Clause 21. A system comprising one or more processors to control, within a simulation that is rendered using one or more light transport simulation algorithms, one or more operations of an ego-machine based at least on a representation of whether a child is present in at least one occupant slot of one or more occupant slots, the representation of whether the child is present generated based at least on executing slot-dependent logic to combine two or more types of detection results representative of occupant presence.

Clause 22. The system of clause 21, wherein the simulation is generated, at least in part, using a three-dimensional (3D) content collaboration platform for 3D assets.

Clause 23. The system of clause 22, wherein the 3D content collaboration platform for 3D assets uses OpenUSD.

Clause 24. One or more processors comprising processing circuitry to obtain two or more types of detections representing or derived from different sensor modalities of an ego-machine.

Clause 25. The one or more processors of clause 24, wherein the processing circuitry is further to generate a representation of whether one or more conditions are present based at least on fusion logic of a base control stack of a compute platform of the ego-machine combining the two or more types of detections using one or more configurable parameters of the fusion logic.

Clause 26. The one or more processors of clause 24 or 25, wherein the processing circuitry is further to execute one or more operations of the ego-machine based at least on the representation of whether at least one condition of the one or more conditions is present.

Clause 27. The one or more processors of clause 24, 25 or 26, wherein the two or more types of detections represent at least one of sensor data or deep learning perception data.

Clause 28. The one or more processors of clause 24, 25 or 26, wherein the one or more configurable parameters of the fusion logic comprise one or more configurable weights, and the combining of the two or more types of detections generates a fused detection confidence based at least on weighting detection confidences of the two or more types of detections using the one or more configurable weights.

Clause 29. The one or more processors of clause 24, 25 or 26, wherein the one or more configurable parameters of the fusion logic comprise one or more configurable weights associated with one or more occupant slots.

Clause 30. The one or more processors of clause 24, 25 or 26, wherein the one or more configurable parameters of the fusion logic toggle false positive suppression logic.

Clause 31. The one or more processors of clause 24, 25 or 26, wherein the one or more configurable parameters of the fusion logic toggle false negative suppression logic.

Clause 32. The one or more processors of clause 24, 25 or 26, wherein the combining the two or more types of detections comprises using a first type of detection of the two or more types of detections corresponding to a first sensor modality of the different sensor modalities to correct a second type of detection of the two or more types of detections corresponding to a second sensor modality of the different sensor modalities.

Clause 33. The one or more processors of clause 24, 25 or 26, wherein the one or more configurable parameters of the fusion logic comprise one or more configurable detection confidence thresholds.

Clause 34. The one or more processors of clause 24, 25 or 26, wherein the one or more configurable parameters of the fusion logic comprise one or more configurable detection confidence thresholds associated with one or more occupant slots.

Clause 35. The one or more processors of clause 24, 25 or 26, wherein the combining of the two or more types of detections executes in the base control stack below an application layer of the compute platform.

Clause 36. The one or more processors of clause 24, 25 or 26, wherein the processing circuitry is further to generate the representation of whether the one or more conditions are present based on at least one or more of child presence detection, hands-on-wheel detection, body size detection, abnormal pose detection, heart rate monitoring, or liveness detection.

Clause 37. The one or more processors of clause 24, 25 or 26, wherein the representation of whether the one or more conditions are present comprises two or more types of unified detection results generated based at least on the combining of the two or more types of detections using the one or more configurable parameters of the fusion logic.

Clause 38. The one or more processors of clause 24, 25 or 26, wherein the one or more operations of the ego-machine comprise at least one of issuing an audible or visual alert, adjusting one or more in-vehicle infotainment settings, or activating one or more safety systems.

Clause 39. The one or more processors of clause 24, 25 or 26, wherein the one or more processors are comprised in at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporates one or more machine learning models deployed in a service or microservice along with an OS-level virtualization package (e.g., a container).

Clause 40. A method comprising generating a representation of whether one or more conditions are present based at least on one or more fusion operations in a control stack of a compute platform of an ego-machine combining two or more types of detections using one or more configurable parameters of the one or more fusion operations.

Clause 41. The method of clause 40, further comprising executing one or more operations of the ego-machine based at least on the representation of whether the one or more conditions are present.

Clause 42. The method of clause 40 or 41, wherein the one or more configurable parameters of the one or more fusion operations comprise one or more configurable weights, and the combining of the two or more types of detections generates a fused detection confidence based at least on the one or more configurable weights.

Clause 43. The method of clause 40 or 41, wherein the method is performed by at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporate one or more machine learning models deployed in a service or microservice along with an OS-level virtualization package (e.g., a container).

Clause 44. A system comprising one or more processors to control, within a simulation that is rendered using one or more light transport simulation algorithms, one or more operations of an ego-machine based at least on a representation of whether one or more conditions are present, the representation of whether the one or more conditions are present being generated based at least on one or more fusion operations executed using a control stack of a compute platform associated with the ego-machine, and combining two or more types of detections using one or more configurable parameters of the one or more fusion operations of the base control stack.

Clause 45. The system of clause 44, wherein the simulation is generated, at least in part, using a three-dimensional (3D) content collaboration platform for 3D assets.

Clause 46. The system of clause 45, wherein the 3D content collaboration platform for 3D assets uses OpenUSD.

Clause 47. One or more processors comprising processing circuitry to receive two or more types of detection results representative of presence of one or more detected occupants in one or more occupant slots in an interior space.

Clause 48. The one or more processors of clause 47, wherein the processing circuitry is further to generate a representation of whether a child is present in at least one occupant slot of the one or more occupant slots based at least on fusion logic of a compute platform of an ego-machine combining the two or more types of detection results using one or more configurable parameters of the fusion logic.

Clause 49. The one or more processors of clause 47 or 48, wherein the processing circuitry is further to execute one or more operations based at least on the representation of whether the child is present.

Clause 50. The one or more processors of clause 47, 48 or 49, wherein the one or more configurable parameters comprise one or more temporal offsets aligning corresponding frames of the two or more types of detection results.

Clause 51. The one or more processors of clause 47, 48 or 49, wherein the one or more configurable parameters comprise one or more configurable weights, and the combining of the two or more types of detection results generates a unified detection result based at least on weighting at least one of the two or more types of detection results using the one or more configurable weights.

Clause 52. The one or more processors of clause 47, 48 or 49, wherein the one or more configurable parameters instruct whether to apply logic that determines whether to overwrite at least one of an inconclusive child presence classification or a classification indicating presence of an adult based at least on RADAR liveness detection indicating a human-like signature and child seat detection indicating presence of a detected child seat.

Clause 53. The one or more processors of clause 47, 48 or 49, wherein the one or more configurable parameters comprise a configurable RADAR liveness confidence threshold, and the combining of the two or more types of detection comprises evaluating whether the at least one occupant slot is occupied by a live occupant using the configurable RADAR liveness confidence threshold.

Clause 54. The one or more processors of clause 47, 48 or 49, wherein the one or more configurable parameters comprise a configurable child seat detection confidence threshold, and the combining of the two or more types of detection comprises evaluating whether the at least one occupant slot is occupied by a child seat using the configurable child seat detection confidence threshold.

Clause 55. The one or more processors of clause 47, 48 or 49, wherein the one or more configurable parameters instruct whether to apply logic that determines whether to overwrite at least one of an inconclusive child presence classification or a classification indicating presence of an adult based at least on camera presence or occupancy detection indicating presence of an occupant and child seat detection indicating presence of a child seat.

Clause 56. The one or more processors of clause 47, 48 or 49, wherein the one or more configurable parameters instruct whether to apply logic that determines whether to overwrite an inconclusive child presence classification based at least on camera presence or occupancy detection indicating presence of an occupant and at least one of RADAR classification or camera age estimation indicating presence of an adult in the at least one occupant slot.

Clause 57. The one or more processors of clause 47, 48 or 49, wherein the one or more configurable parameters comprise a configurable camera presence or occupancy detection confidence threshold, and the combining of the two or more types of detection comprises evaluating whether image data indicates the at least one occupant slot is occupied with at least the configurable camera presence or occupancy detection confidence threshold.

Clause 58. The one or more processors of clause 47, 48 or 49, wherein the one or more configurable parameters comprise a configurable age estimation confidence threshold, and the combining of the two or more types of detection comprises evaluating whether an age or age range estimated based at least on image data indicates the at least one occupant slot is occupied by at least one of an adult or a child with at least the configurable age estimation confidence threshold.

Clause 59. The one or more processors of clause 47, 48 or 49, wherein the one or more configurable parameters instruct whether to apply logic that determines whether to overwrite a classification indicating presence of the child based at least on evaluating whether an age or age range estimated based at least on image data indicates the at least one occupant slot is occupied by an adult.

Clause 60. The one or more processors of clause 47, 48 or 49, wherein the one or more processors are comprised in at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporate one or more machine learning models deployed in a service or microservice along with an OS-level virtualization package (e.g., a container).

Clause 61. A method comprising generating a representation of whether a child is present in at least one occupant slot of one or more occupant slots based at least on one or more fusion operations of a compute platform of an ego-machine combining two or more types of detections using one or more configurable parameters of the one or more fusion operations.

Clause 62. The method of clause 61, further comprising executing one or more operations of the ego-machine based at least on the representation of whether a child is present..

Clause 63. The method of clause 61 or 62, wherein the one or more configurable parameters comprise one or more temporal offsets aligning corresponding frames of the two or more types of detection results.

Clause 64. The method of clause 61 or 62, wherein the one or more configurable parameters comprise one or more configurable weights, and the combining of the two or more types of detection results generates a unified detection result based at least on weighting at least one of the two or more types of detection results using the one or more configurable weights.

Clause 65. The method of clause 61 or 62, wherein the one or more configurable parameters instruct whether to apply logic that determines whether to overwrite at least one of an inconclusive child presence classification or a classification indicating presence of an adult based at least on RADAR liveness detection indicating a human-like signature and child seat detection indicating presence of a detected child seat.

Clause 66. The method of clause 61 or 62, wherein the method is performed by at least one of: a control system for an autonomous or semi-autonomous machine; a perception system for an autonomous or semi-autonomous machine; a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system implemented using a robot; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; a system implemented at least partially using cloud computing resources; a system using or deploying one or more inference microservices; or a system that incorporate one or more machine learning models deployed in a service or microservice along with an OS-level virtualization package (e.g., a container).

Clause 67. A system comprising one or more processors to control, within a simulation that is rendered using one or more light transport simulation algorithms, one or more operations of a simulated ego-machine based at least on a representation of whether a child is present in at least one occupant slot of one or more occupant slots of the simulated ego-machine, the representation of whether the child is present generated based at least on one or more fusion operations of a compute platform associated with the simulated ego-machine combining two or more types of detections using one or more configurable parameters of the one or more fusion operations of the base control stack.

Clause 68. The system of clause 67, wherein the simulation is generated, at least in part, using a three-dimensional (3D) content collaboration platform for 3D assets.

Clause 69. The system of clause 68, wherein the 3D content collaboration platform for 3D assets uses OpenUSD.

What is claimed is:

1. One or more processors comprising processing circuitry to:
receive two or more types of detection results representative of presence of one or more detected occupants in one or more occupant slots in an interior space;
generate a representation of whether a child is present in at least one occupant slot of the one or more occupant slots based at least on fusion logic of a compute platform of an ego-machine combining the two or more types of detection results using one or more configurable parameters of the fusion logic; and
execute one or more operations based at least on the representation of whether the child is present.

2. The one or more processors of claim 1, wherein the one or more configurable parameters comprise one or more temporal offsets aligning corresponding frames of the two or more types of detection results.

3. The one or more processors of claim 1, wherein the one or more configurable parameters comprise one or more configurable weights, and the combining of the two or more types of detection results generates a unified detection result based at least on weighting at least one of the two or more types of detection results using the one or more configurable weights.

4. The one or more processors of claim 1, wherein the one or more configurable parameters instruct whether to apply logic that determines whether to overwrite at least one of an inconclusive child presence classification or a classification indicating presence of an adult based at least on RADAR liveness detection indicating a human-like signature and child seat detection indicating presence of a detected child seat.

5. The one or more processors of claim 1, wherein the one or more configurable parameters comprise a configurable RADAR liveness confidence threshold, and the combining of the two or more types of detection comprises evaluating whether the at least one occupant slot is occupied by a live occupant using the configurable RADAR liveness confidence threshold.

6. The one or more processors of claim 1, wherein the one or more configurable parameters comprise a configurable child seat detection confidence threshold, and the combining of the two or more types of detection comprises evaluating whether the at least one occupant slot is occupied by a child seat using the configurable child seat detection confidence threshold.

7. The one or more processors of claim 1, wherein the one or more configurable parameters instruct whether to apply logic that determines whether to overwrite at least one of an inconclusive child presence classification or a classification indicating presence of an adult based at least on camera presence or occupancy detection indicating presence of an occupant and child seat detection indicating presence of a child seat.

8. The one or more processors of claim 1, wherein the one or more configurable parameters instruct whether to apply logic that determines whether to overwrite an inconclusive child presence classification based at least on camera presence or occupancy detection indicating presence of an occupant and at least one of RADAR classification or camera age estimation indicating presence of an adult in the at least one occupant slot.

9. The one or more processors of claim 1, wherein the one or more configurable parameters comprise a configurable camera presence or occupancy detection confidence threshold, and the combining of the two or more types of detection comprises evaluating whether image data indicates the at least one occupant slot is occupied with at least the configurable camera presence or occupancy detection confidence threshold.

10. The one or more processors of claim 1, wherein the one or more configurable parameters comprise a configurable age estimation confidence threshold, and the combining of the two or more types of detection comprises evaluating whether an age or age range estimated based at least on image data indicates the at least one occupant slot is occupied by at least one of an adult or a child with at least the configurable age estimation confidence threshold.

11. The one or more processors of claim 1, wherein the one or more configurable parameters instruct whether to apply logic that determines whether to overwrite a classification indicating presence of the child based at least on evaluating whether an age or age range estimated based at least on image data indicates the at least one occupant slot is occupied by an adult.

12. The one or more processors of claim 1, wherein the one or more processors are comprised in at least one of:
a control system for an autonomous or semi-autonomous machine;
a perception system for an autonomous or semi-autonomous machine;
a system for performing simulation operations;
a system for performing digital twin operations;
a system for performing light transport simulation;
a system for performing collaborative content creation for 3D assets;
a system for performing deep learning operations;
a system for performing remote operations;
a system for performing real-time streaming;
a system for generating or presenting one or more of augmented reality content, virtual reality content, or mixed reality content;
a system implemented using an edge device;
a system implemented using a robot;
a system for performing conversational AI operations;
a system implementing one or more language models;
a system implementing one or more large language models (LLMs);
a system implementing one or more vision language models (VLMs);
a system implementing one or more multi-modal language models;
a system for generating synthetic data;
a system for generating synthetic data using AI;
a system for performing one or more generative AI operations;
a system incorporating one or more virtual machines (VMs);
a system implemented at least partially in a data center;
a system implemented at least partially using cloud computing resources;
a system using or deploying one or more inference microservices; or
a system that incorporate one or more machine learning models deployed in a service or microservice along with an OS-level virtualization package (e.g., a container).

13. A method comprising:
generating a representation of whether a child is present in at least one occupant slot of one or more occupant slots based at least on one or more fusion operations of a compute platform of an ego-machine combining two or more types of detections using one or more configurable parameters of the one or more fusion operations; and
executing one or more operations of the ego-machine based at least on the representation of whether a child is present.

14. The method of claim 13, wherein the one or more configurable parameters comprise one or more temporal offsets aligning corresponding frames of the two or more types of detection results.

15. The method of claim 13, wherein the one or more configurable parameters comprise one or more configurable weights, and the combining of the two or more types of detection results generates a unified detection result based at least on weighting at least one of the two or more types of detection results using the one or more configurable weights.

16. The method of claim 13, wherein the one or more configurable parameters instruct whether to apply logic that determines whether to overwrite at least one of an inconclusive child presence classification or a classification indicating presence of an adult based at least on RADAR liveness detection indicating a human-like signature and child seat detection indicating presence of a detected child seat.

17. The method of claim 13, wherein the method is performed by at least one of:
a control system for an autonomous or semi-autonomous machine;
a perception system for an autonomous or semi-autonomous machine;
a system for performing simulation operations;
a system for performing digital twin operations;
a system for performing light transport simulation;
a system for performing collaborative content creation for 3D assets;
a system for performing deep learning operations;
a system for performing remote operations;
a system for performing real-time streaming;
a system for generating or presenting one or more of augmented reality content, virtual reality content, or mixed reality content;
a system implemented using an edge device;
a system implemented using a robot;
a system for performing conversational AI operations;
a system implementing one or more language models;
a system implementing one or more large language models (LLMs);
a system implementing one or more vision language models (VLMs);
a system implementing one or more multi-modal language models;
a system for generating synthetic data;
a system for generating synthetic data using AI;
a system for performing one or more generative AI operations;
a system incorporating one or more virtual machines (VMs);
a system implemented at least partially in a data center;
a system implemented at least partially using cloud computing resources;
a system using or deploying one or more inference microservices; or
a system that incorporate one or more machine learning models deployed in a service or microservice along with an OS-level virtualization package (e.g., a container).

18. A system comprising one or more processors to control, within a simulation that is rendered using one or more light transport simulation algorithms, one or more operations of a simulated ego-machine based at least on a representation of whether a child is present in at least one occupant slot of one or more occupant slots of the simulated ego-machine, the representation of whether the child is present generated based at least on one or more fusion operations of a compute platform associated with the simulated ego-machine combining two or more types of detections using one or more configurable parameters of the one or more fusion operations of the base control stack.

19. The system of claim 18, wherein the simulation is generated, at least in part, using a three-dimensional (3D) content collaboration platform for 3D assets.

20. The system of claim 19, wherein the 3D content collaboration platform for 3D assets uses OpenUSD.
